SEQUENCE LISTING (4 sequences)

SEQ ID NO: 1 - protein, 73 aa, Homo sapiens
APLATELRCQCLQTLQGIHLKNIQSVKVKSPGPHCAQTEVIATLKNGQKACLNPASPMVKKIIEKMLKNGKSN

SEQ ID NO: 2 - protein, 69 aa, Homo sapiens
TELRCQCLQTLQGIHLKNIQSVKVKSPGPHCAQTEVIATLKNGQKACLNPASPMVKKIIEKMLKNGKSN

SEQ ID NO: 3 - protein, 73 aa, Homo sapiens
APLATELRCQCLQTLQGIHLKNIQSVKVKSPGPHCAQTEVIATLKNGQKACLNPASPMVKKIIEKMLKDGKSN

SEQ ID NO: 4 - protein, 69 aa, Homo sapiens
TELRCQCLQTLQGIHLKNIQSVKVKSPGPHCAQTEVIATLKNGQKACLNPASPMVKKIIEKMLKDGKSN

(19) United States
(12) Patent Application Publication
(10) Pub. No.: US 2021/0379113 A1
(43) Pub. Date: Dec. 9, 2021

(54) METHODS FOR HEMATOPOIETIC STEM AND PROGENITOR CELL TRANSPLANT THERAPY

(71) Applicant: Magenta Therapeutics Inc., Cambridge, MA (US)

(72) Inventor: Glen RAFFEL, Cambridge, MA (US)

(21) Appl. No.: 17/290,537

(22) PCT Filed: Oct. 31, 2019
(86) PCT No.: PCT/US2019/059039

§ 371 Date: Apr. 30, 2021

Related U.S. Application Data
(60) Provisional application No. 62/860,866, filed on Jun. 13, 2019.
(60) Provisional application No. 62/753,865, filed on Oct. 31, 2018.

(51) Int. Cl. A61K 35/28; C07K 16/18; A61K 31/255; A61K 31/675; C12N 5/0789

(52) U.S. Cl. CPC A61K 35/28; C07K 16/18; A61K 31/4015; A61K 31/675; C12N 5/0647; A61K 31/255

(57) ABSTRACT

Provided herein are compositions and methods useful for the transplantation of hematopoietic stem and progenitor cells, as well as for preparing patients for receipt of such therapy, such as patients suffering from a variety of inherited metabolic disorders.

### RELATED APPLICATIONS

This application claims priority to U.S. Application Nos. 62/860,866, filed Jun. 13, 2019, and 62/753,865, filed Oct. 31, 2018, the entire contents of each of which are incorporated herein by reference.

### FIELD

The present disclosure relates to compositions and methods useful for the transplantation of hematopoietic stem and progenitor cells, as wet as for preparing patients for receipt of such therapy, for instance, patients suffering from a variety of pathologies, such as inherited metabolic disorders.

### BACKGROUND

Despite advances in the medicinal arts, there remains a demand for treating pathologies of the hematopoietic system, such as diseases of a particular blood cell, metabolic disorders, cancers, and autoimmune conditions, among others. While hematopoietic stem cells have significant therapeutic potential, a limitation that has hindered their use in the clinic has been the difficulty associated with conditioning patients for infusion of populations of hematopoietic stem cells. There is currently a need for compositions and methods for administering such therapy.

### SUMMARY

In some aspects, the present disclosure provides compositions and methods for expanding populations of hematopoietic stem or progenitor cells.

In some aspects, the present disclosure provides compositions and methods for the transplantation of hematopoietic stem or progenitor cells, for instance, for the treatment of various inherited metabolic disorders, such as those described herein.

In some aspects, the present disclosure provides a method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents or reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some aspects, the present disclosure provides a method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents, or reduces the severity of, autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some aspects, the present disclosure provides a method of preventing, or reducing the risk of, autoimmune cytopenia in a patient in need thereof, the method comprising: i) conditioning the patient with a conditioning regimen; and ii) administering to (e.g., infusing into) the patient a population of hematopoietic stem or progenitor cells (e.g., expanded cord blood (e.g., MGTA-456)).

In some aspects, the present disclosure provides a method of preventing, or reducing the risk of, autoimmune cytopenia in a patient in need thereof, the method comprising: i) administering to the patient a prophylactic agent prior to, during, or following transplant with expanded core blood (e.g., MGTA-456); and ii) transplanting the patient with expanded cord blood (e.g., MGTA-456); wherein the prophylactic agent inhibits the production of antibodies in the patient.

In some aspects, the present disclosure provides a method of preventing, or reducing the risk of, autoimmune cytopenia in a patient in need thereof, the method comprising: i) conditioning the patient with a conditioning regimen; and ii) transplanting the patient with expanded cord blood (e.g., MGTA-456); wherein the conditioning regimen does not comprise busulfan plus fludarabine (BuFlu).

In some aspects, the present disclosure provides a method of preparing a patient for hernatopoietic stem or progenitor cell transplantation, the method comprising conditioning the patient with a conditioning regimen.

In some aspects, the present disclosure provides a method (e.g., of administering hematopoietic stem cell transplantation therapy to a patient in need thereof), the method comprising administering to (e.g., infusing into) the patient a population of hematopoietic stem or progenitor cells (e.g., expanded cord blood (e.g., MGTA-456)), wherein the patient has previously been conditioned with a conditioning regimen.

In some aspects, the present disclosure provides a method of administering hematopoietic stem cell transplantation therapy to a patient in need thereof, wherein the patient has previously been conditioned with a conditioning regimen, the method comprising infusing into the patient a population of hematopoietic stem or progenitor cells.

In some aspects, the present disclosure provides a method (e.g., of administering hematopoietic stem cell transplantation therapy to a patient in need thereof), the method comprising: a) conditioning the patient with a conditioning regimen; and b) administering, to (e.g., infusing into) the patient a population of hernatopoietic stem or progenitor cells (e.g., expanded cord blood (e.g., MGTA-456)).

In some aspects, the present disclosure provides a method of administering hernatopoietic stem or progenitor cell transplant therapy to a patient in need thereof, the method comprising: a) conditioning the patient with a conditioning regimen; and b) infusing into the patient a population of hernatopoietic stem or progenitor cells.

In some aspects, the present disclosure provides a method of administering hematopoietic stem or progenitor cell transplant therapy to a patient in need thereof, the method comprising: a) conditioning the patient with a conditioning regimen, wherein the conditioning regimen comprising a1) administering busulfan (Bu); a2) administering cyclophosphamide (Cy); and a3) administering anti-thymocyte globulin (rabbit)(ATG); and b) infusing into the patient a population of hematopoietic stem or progenitor cells.

In some aspects, the present disclosure provides a method of administering hematopoietic stem or progenitor cell transplant therapy to a patient in need thereof, the method comprising: a) conditioning the patient with a conditioning regimen, wherein the conditioning regimen comprising a1) administering busulfan (Bu) prior to administering cyclophosphamide and prior to administering anti-thymocyte globulin (rabbit) (ATG); a2) administering cyclophosphamide (Cy) after administering busulfan (Bu) and simultaneously with administering anti-thymocyte globulin (rabbit) (ATG); and a3) administering anti-thymocyte globulin (rabbit) (ATG) after administering busulfan (Bu) and simultaneously with administering cyclophosphamide (Cy); and b) infusing into the patient a population of hematopoietic stem or progenitor cells.

In some aspects, the present disclosure provides a method of administering hematopoietic stem or progenitor cell transplant therapy to a patient in need thereof, the method comprising: a) conditioning the patient with a conditioning regimen, wherein the conditioning regimen comprising a1) administering busulfan (Bu) at days −9 to −6 prior to infusing into the patient a population of hematopoietic stem or progenitor cells; a2) administering cyclophosphamide (Cy) at days −5 to −2 prior to infusing into the patient a population of hernatopoietic stem or progenitor cells; and a3) administering anti-thymocyte globulin (rabbit)(ATG) at days −5 to −2 prior to infusing into the patient a population of hematopoietic stem or progenitor cells; and b) infusing into the patient a population of hematopoietic stem or progenitor cells at day 0.

In some aspects, the present disclosure provides a method of administering hematopoietic stem or progenitor cell transplant therapy to a patient in need thereof, the method comprising: a) conditioning the patient with a conditioning regimen, wherein the conditioning regimen comprising a1) administering busulfan (Bu) at a dose wherein the plasma exposure as measured by cumulative AUC is maintained within a range of 74-82 mg*hr/L for 4 consecutive days at days −9 to −6 prior to infusing into the patient a population of hernatopoietic stem or progenitor cells; a2) administering cyclophosphamide (Cy) at a dose of 50 mg/kg/day for 4 consecutive days at days −5 to −2 prior to infusing into the patient a population of hematopoietic stem or progenitor cells; and a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and b) infusing into the patient a population of hematopoietic stem or progenitor cells at day 0.

In some aspects, the present disclosure provides a method of administering hematopoietic stem or progenitor cell transplant therapy to a patient in need thereof, the method comprising: a) conditioning the patient with a conditioning regimen, wherein the conditioning regimen comprising a1) administering busulfan (Bu) at a dose wherein the plasma exposure as measured by steady state concentration (Css) is maintained within a range of 770-850 ng/mL for 4 consecutive days at days −9 to −6 prior to infusing into the patient a population of hematopoietic stem or progenitor cells; a2) administering cyclophosphamide (Cy) at a dose of 50 mg/kg/day for 4 consecutive days at days −5 to 2 prior to infusing into the patient a population of hematopoietic stem or progenitor cells; and a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and b) infusing into the patient a population of hematopoietic stem or progenitor cells at day 0.

In some aspects, the present disclosure provides a method (e.g., of administering hematopoietic stem or progenitor cell transplant therapy to a patient in need thereof), the method comprising: (a) expanding, ex vivo, a population of hematopoietic stem or progenitor cells (e.g., CD34+ cells) comprising no more than 1×10⁸ CD34+ cells; and (b) infusing into the patient the expanded population of hematopoietic stem or progenitor cells, or progeny thereof.

In some aspects, the present disclosure provides a method (e.g., of administering hematopoietic stem or progenitor cell transplant therapy to a patient in need thereof), the method comprising infusing into the patient a population of hematopoietic stem or progenitor cells that have been expanded ex vivo, wherein the population, prior to expansion, comprises no more than 1×10⁸ CD34+ cells.

In some aspects, the present disclosure provides a method of treating or preventing a disorder (e.g., a stem cell disorder) in a patient in need thereof, the method comprising administering to (e.g., infusing into) the patient a population of hematopoietic stem or progenitor cells (e.g., expanded cord blood (e.g., MGTA-456)), wherein the patient has previously been conditioned with a conditioning regimen.

In some aspects, the present disclosure provides a method of treating or preventing a disorder (e.g., a stem cell disorder) in a patient in need thereof, the method comprising: a) conditioning the patient with a conditioning regimen; and b) administering to (e.g., infusing into) the patient a population of hematopoietic stem or progenitor cells (e.g., expanded cord blood (e.g., MGTA-456)).

In some aspects, the present disclosure provides a method of treating a stem cell disorder in a patient (e.g., a human patient), the method comprising administering hematopoietic stem or progenitor cell transplant therapy to the patient in accordance with the method of any of the foregoing aspects or embodiments.

In some aspects, the present disclosure provides a population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the risk of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

In some aspects, the present disclosure provides a population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the severity of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

In some aspects, the present disclosure provides a combination of a conditioning regimen and a population of hematopoietic stem or progenitor cells for preventing, or reducing the risk of, autoimmune cytopenia in a patient in need thereof, wherein the patient is conditioned with the conditioning regimen prior to being administered with the population of hematopoietic stem or progenitor cells.

In some aspects, the present disclosure provides a combination of a conditioning regimen and an expanded cord blood for preventing, or reducing the risk of, autoimmune cytopenia in a patient in need thereof, wherein the patient is conditioned with the conditioning regimen prior to being administered with the expanded cord blood, and wherein the conditioning regimen does not comprise busulfan plus fludarabine (BuFlu).

In some aspects, the present disclosure provides a population of hematopoietic stem or progenitor cells for being administered to a patient, wherein the patient is conditioned with a conditioning regimen prior to the administration of the population of hematopoietic stem or progenitor cells.

In some aspects, the present disclosure provides a population of hematopoietic stem or progenitor cells for administering hematopoietic stem cell transplantation therapy to a patient in need thereof, wherein the is conditioned with a conditioning regimen prior to infusing into the patient the population of hematopoietic stem or progenitor cells.

In some aspects, the present disclosure provides a conditioning regimen (e.g., prophylactic agent) for preventing or reducing the risk of autoimmune cytopenia in a patient in need thereof, wherein the conditioning regimen (e.g., prophylactic agent) is administered to the patient prior to, during, or following ttransplanting the patient with expanded cord blood; and wherein the conditioning regimen (e.g., prophylactic agent) inhibits the production of antibodies in the patient.

In some aspects, the present disclosure provides a conditioning regimen for preparing a patient for hematopoietic stem or progenitor cell transplantation.

In some aspects, the present disclosure provides a population of hematopoietic stem or progenitor cells for administering hematopoietic stem or progenitor cell transplant therapy to a patient in need thereof, wherein the population of hematopoietic stem or progenitor cells that have been expanded ex vivo, wherein the population, prior to expansion, comprises no more than 1×10⁸ CD34+ cells.

In some aspects, the present disclosure provides a population of hematopoietic stem or progenitor cells for treating a stem cell disorder in a patient.

In some aspects, the present disclosure provides a kit comprising a plurality of hematopoietic stem or progenitor cells and a package insert that instructs a user to perform the method of any of the above aspects or embodiments.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

In the case of conflict between the chemical structures and names of the compounds disclosed herein, the chemical structures will control.

Other features and advantages of the disclosure will be apparent from the following detailed description and claims.

### DETAILED DESCRIPTION

Provided herein are compositions and methods for administering hematopoietic stem cell transplantation therapy to a patient, such as a human patient suffering from one or more stem cell disorders as described herein. Using the compositions and methods described herein, the patient may be administered one or more conditioning agents, such as one or more nonmyeloablative conditioning agents, so as to deplete a population of endogenous hematopoietic stem or progenitor cells in a stem cell niche within the patient. A population of hematopoietic stem or progenitor cells may then be infused into the patient, and the hematopoietic stem or progenitor cells may then migrate to the stem cell niche that has been partially vacated by the nonmyeloablative conditioning regimen.

Thus, provided herein are methods of treating various hematological disorders, as the hematopoietic stem and progenitor cells infused into the patient may go on to populate one or more of the hematopoietic lineages, thereby replenishing a population of cells that is deficient or defective within the patient.

The sections that follow describe, in further detail, the compositions and methods that can be used to effectuate the conditioning of a patient in preparation for hematopoietic stem cell transplantation, as well as compositions and methods for conducting hematopoietic stem or progenitor cell transplantation.

### Definitions

As used herein, the term “about” refers to a value that is within 10% above or below the value being described. For example, the term “about 5 nM” indicates a range of from 4.5 nM to 5.5 nM.

As used herein, the term “alkylating agent” or “alkylating antineoplastic agent” refers to an alkylating agent that attaches an alkyl group (CₙH₂ₙ₊₁) to DNA. In some embodiments, the alkyl group is attached to the guanine base of DNA, at the number 7 nitrogen atom of the purine ring.

As used herein, the term “purine analog” refers to an antimetabolite that mimics the structure of metabolic purines. Exemplary purine analogs include, but are not limited to, azathioprine, mercaptopurine, thiopurines (e.g., thioguanine), fludarabine (Flu), pentostatin, methotrexate, and cladribine (2-CDA).

As used herein, the term “chimerism” refers to a state in which one or more cells from a donor are present and functioning in a recipient or host, such as a patient that is receiving or has received hematopoietic stem or progenitor cell transplant therapy as described herein. Recipient tissue exhibiting “chimerism” may contain donor cells only (complete chimerism), or it may contain both donor and host cells (mixed chimerism). “Chimerism” as used herein may refer to either transient or stable chimerism. In some embodiments, the mixed chimerism may be MHC- or HLA-matched mixed chimerism. In certain embodiments, the mixed chimerism may be MHC- or HLA-mismatched mixed chimerism.

As used herein, the terms “condition” and “conditioning” refer to processes by which a patient is prepared for receipt of a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation.

According to the methods described herein, a patient may be conditioned for hematopoietic stem cell transplant therapy by administration to the patient of a non-myeloablative conditioning regimen, such as by way of an antibody or antigen-binding fragment thereof capable of binding an antigen expressed by hematopoietic stem cells. As described herein, the antibody may be covalently conjugated to a cytotoxin so as to form a drug-antibody conjugate. Administration of an antibody, antigen-binding fragment thereof, or drug-antibody conjugate capable of binding one or more hematopoietic stem or progenitor cell antigens to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in table A below.

TABLE A
Representative physicochemical properties
of naturally-occurring amino acids

| | | | | Electrostatic | |
|---|---|---|---|---|---|
| | 3 | 1 | | character at | |
| | Letter | Letter | Side-chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium at | |
| | | | | pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A3: 50-100 is small, 100-150 is intermediate, 150-200 is large, and >200 is bulky

From this table it is appreciated that the conservative amino acid families include, e.g., (I) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, “CRU (competitive repopulating unit)” refers to a unit of measure of long-term engrafting stem cells, which can be detected after in-vivo transplantation.

As used herein, the term “comparable method” refers to a method with comparable (e.g., the same) parameters and/or steps, as the method being compared (e.g., a method of the present disclosure). In some embodiments, the “comparable method” is a method in which the patient is conditioned prior to receiving the population of expanded hematopoietic stem or progenitor cells by a conditioning regimen not comprising administering to the patient all of busulfan (Bu), cyclophosphamide (Cy), and anti-thymocyte globulin (rabbit) (rATG). In some embodiments, the “comparable method” is a method in which the patient is conditioned prior to receiving the population of expanded hematopoietic stem or progenitor cells by a conditioning regimen comprising administering to the patient busulfan (Bu) and fludarabine (Flu). In some embodiments, the “comparable method” is a method in which the patient is conditioned prior to receiving the population of expanded hematopoietic stem or progenitor cells by a conditioning regimen comprising administering to the patient busulfan (Bu), fludarabine (Flu), and ATG (e.g., rATG).

As used herein, the term “donor” refers to a subject, such as a mammalian subject (e.g., a human subject) from which one or more cells are isolated prior to administration of the cells, or progeny thereof, into a recipient. The one or more cells may be, for example, a population of hematopoietic stem or progenitor cells.

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. In some embodiments, one non-limiting example of engraftment is achievement of an absolute neutrophil count (ANC) of greater than or equal to 0.5×10⁹/L for 3 consecutive days. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

As used herein, the term “hematopoietic progenitor cells” includes pluripotent cells capable of differentiating into several cell types of the hematopoietic system, including, without limitation, granulocytes, monocytes, erythrocytes, megakaryocytes, B-cells and T-cells, among others. Hematopoietic progenitor cells are committed to the hematopoietic cell lineage and generally do not self-renew. Hematopoietic progenitor cells can be identified, for example, by expression patterns of cell surface antigens, and include cells having the following immunophenotype: Lin− KLS+ Flk2-CD34+. Hematopoietic progenitor cells include short-term hematopoietic stem cells, mufti-potent progenitor cells, common myeloid progenitor cells, granulocyte-monocyte progenitor cells, and megakaryocyte-erythrocyte progenitor cells. The presence of hematopoietic progenitor cells can be determined functionally, for instance, by detecting colony-forming unit cells, e.g., in complete methylcellulose assays, or phenotypically through the detection of cell surface markers using flow cytometry and cell sorting assays described herein and known in the art.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells. CD34⁺ cells are immature cells that express the CD34 cell surface marker. In humans, CD34+ cells are believed to include a subpopulation of cells with the stem cell properties defined above, whereas in mice, HSCs are CD34-. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48-, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the terms “Major histocompatibility complex antigens” (“MHC”, also referred to as “human leukocyte antigens” (“HLA”) in the context of humans) refer to proteins expressed on the cell surface that confer a unique antigenic identity to a cell. MHC/HLA antigens are target molecules that are recognized by T cells and NK cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells. Both have been implicated in the rejection of transplanted organs. An important aspect of the HLA gene system is its polymorphism. Each gene, MHC class I (A, B and C) and MHC class II (DP, DQ and DR) exists in different alleles. For example, two unrelated individuals may carry class I HLA-B, genes B5, and Bw41, respectively. Allelic gene products differ in one or more amino acids in the a and/or p domain(s). Large panels of specific antibodies or nucleic acid reagents are used to type HLA haplotypes of individuals, using leukocytes that express class I and class II molecules. The genes commonly used for HLA typing are the six MHC Class I and Class II proteins, two alleles for each of HLA-A; HLA-B and HLA-DR. The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the term “aryl hydrocarbon receptor (AHR) modulator” refers to an agent that causes or facilitates a qualitative or quantitative change, alteration, or modification in one or more processes, mechanisms, effects, responses, functions, activities or pathways mediated by the AHR receptor. Such changes mediated by an AHR modulator, such as an inhibitor or a non-constitutive agonist of the AHR described herein, can refer to a decrease or an increase in the activity or function of the AHR, such as a decrease in, inhibition of, or diversion of, constitutive activity of the AHR.

An “AHR antagonist” refers to an AHR inhibitor that does not provoke a biological response itself upon specifically binding to the AHR polypeptide or polynucleotide encoding the AHR, but blocks or dampens agonist-mediated or ligand-mediated responses, i.e., an AHR antagonist can bind but does not activate the AHR polypeptide or polynucleotide encoding the AHR, and the binding disrupts the interaction, displaces an AHR agonist, and/or inhibits the function of an AHR agonist. Thus, as used herein, an AHR antagonist does not function as an inducer of AHR activity when bound to the AHR, i.e., they function as pure AHR inhibitors.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer from or otherwise be affected by a metabolic disorder selected from the group consisting of glycogen storage diseases, mucopolysaccharidoses, Gaucher disease, Hurler disease, sphingolipidoses, metachromatic leukodystrophy, globoid cell leukodystrophy, cerebral adrenoleukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the terms “mobilize” and “mobilization” refer to processes by which a population of hematopoietic stem or progenitor cells is released from a stem cell niche, such as the bone marrow of a subject, into circulation in the peripheral blood. Mobilization of hematopoietic stem and progenitor cells can be monitored, for instance, by assessing the quantity or concentration of hematopoietic stem or progenitor cells in a peripheral blood sample isolated from a subject. For example, the peripheral blood sample may be withdrawn from the subject, and the quantity or concentration of hematopoietic stem or progenitor cells in the peripheral blood sample may subsequently be assessed, following the administration of a hematopoietic stem or progenitor cell mobilization regimen to the subject. The mobilization regimen may include, for instance, a CXCR4 antagonist, such as a CXCR4 antagonist described herein (e.g., plenxafor or a variant thereof), and a CXCR2 agonist, such as a CXCR2 agonist described herein (e.g., Gro-β or a variant thereof, such as a truncation of Gro-β, for instance, Gro-β T). The quantity or concentration of hematopoietic stem or progenitor cells in the peripheral blood sample isolated from the subject following administration of the mobilization regimen may be compared to the quantity or concentration of hematopoietic stem or progenitor cells in a peripheral blood sample isolated from the subject prior to administration of the mobilization regimen. An observation that the quantity or concentration of hematopoietic stem or progenitor cells has increased in the peripheral blood of the subject following administration of the mobilization regimen is an indication that the subject is responding to the mobilization regimen, and that hematopoietic stem and progenitor cells have been released from one or more stem cell niches, such as the bone marrow, into peripheral blood circulation.

As used herein, the term “non-myeloablative” refers to a conditioning regimen that does not eliminate substantially all hematopoietic cells of host origin.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the phrase “stem cell disorder” broadly refers to any disease, disorder, or condition that may be treated or cured by engrafting or transplanting a population of hematopoietic stem or progenitor cells in a target tissue within a patient. For example, Type I diabetes has been shown to be cured by hematopoietic stem cell transplant, along with various other disorders. Diseases that can be treated by infusion of hematopoietic stem or progenitor cells into a patient include, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated by transplantation of hematopoietic stem and progenitor cells as described herein include blood disorders (e.g., sickle cell anemia) and autoimmune disorders, such as scleroderma, multiple sclerosis, ulcerative colitis, and Chrohn's disease. Additional diseases that may be treated using hematopoietic stem and progenitor cell transplant therapy include cancer, such as a cancer described herein. Stem cell disorders include a malignancy, such as a neuroblastoma or a hematologic cancers, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using hematopoietic stem or progenitor cell transplant therapy include myelodysplastic syndrome. In some embodiments, the patient has or is otherwise affected by a metabolic storage disorder. For example, the patient may suffer from or otherwise be affected by a metabolic disorder selected from the group consisting of glycogen storage diseases, mucopolysaccharidoses, Gaucher disease, Hurler disease, sphingolipidoses, metachromatic leukodystrophy, globoid cell leukodystrophy, or cerebral adrenoleukodystrophy or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem or progenitor cell transplant therapy.

As used herein, the terms “subject” and “patient” refer to an organism, such as a human, that receives treatment for a particular disease or condition as described herein. For instance, a patient, such as a human patient, that is in need of hematopoietic stem cell transplantation may receive treatment that includes a population of hematopoietic stem cells so as to treat a stem cell disorder, such as a cancer, autoimmune disease, or metabolic disorder described herein.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder or to promote a beneficial phenotype in the patient being treated. Beneficial or desired clinical results include, but are not limited to, promoting the engraftment of exogenous hematopoietic cells in a patient following hematopoietic stem or progenitor cell transplant therapy. Additional beneficial results include an increase in the cell count or relative concentration of hematopoietic stem cells in a patient in need of a hematopoietic stem or progenitor cell transplant following administration of an exogenous hematopoietic stem or progenitor cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following and subsequent hematopoietic stem cell transplant therapy. Additional beneficial results may include the reduction in quantity of a disease-causing cell population, such as a population of cancer cells or autoimmune cells.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of peptides and proteins, such as those described herein, include plasmids that contain regulatory sequences, such as promoter and enhancer regions, which direct gene transcription. Other useful vectors for expression of peptides and proteins described herein contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “alkyl” refers to a straight- or branched-chain alkyl group having, for example, from 1 to 20 carbon atoms in the chain. Examples of alkyl groups include methyl, ethyl, n-propyl, isopropyl, butyl, isobutyl, sec-butyl, tert-butyl, pentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. Examples of alkenyl groups include vinyl, propenyl, isopropenyl, butenyl, tert-butylenyl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain. Examples of alkynyl groups include propargyl, butynyl, pentynyl, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocyloalkyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members.

As used herein, the term “heterocycloalkylene” refers to a divalent heterocyclolalkyl group. The divalent positions may be on the same or different atoms within the ring structure.

As used herein, the term “aryl” refers to a monocyclic or multicyclic aromatic ring system containing, for example, from 6 to 19 carbon atoms. Aryl groups include, but are not limited to, phenyl, fluorenyl, naphthyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group. Heteroaryl groups include pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyndinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term ‘heteroarylene’ refers to a divalent heteroaryl group. The divalent positions may be on the same or different atoms. The divalent positions may be one or more heteroatoms.

Unless otherwise constrained by the definition of the individual substituent, the foregoing chemical moieties, such as “alkyl”, “alkylene”, “heteroalkyl”, “heteroalkylene”, “alkenyl, alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. As used herein, the term “optionally substituted” refers to a compound or moiety containing one or more (for example, 1, 2, 3, 4, 5, 6, 7, 8, 9, 10, or more) substituents, as permitted by the valence of the compound or moiety or a site thereof, such as a substituent selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

As used herein, the term “optionally substituted” refers to a chemical moiety that may have one or more chemical substituents, as valency permits, such as C1-4 alkyl, C2-4 alkenyl, C2-4 alkynyl, C3-10 cycloalkyl, C3-10 heterocycloalkyl, aryl, alkylaryl, heteroaryl, alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

In accordance with the application, any of the aryls, substituted aryls, heteroaryls and substituted heteroaryls described herein, can be any aromatic group.

The terms “hal,” “halo,” and “halogen.” as used herein, refer to an atom selected from fluorine, chlorine, bromine and iodine.

As described herein, compounds of the application and moieties present in the compounds may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to: —F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C_{1,2}-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH-aryl, —OCONH-heteroaryl, —OCONH— heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

Where the number of any given substituent is not specified, there may be one or more substituents present. For example, “halo-substituted C1-4 alkyl” may include one or more of the same or different halogens.

When the compounds described herein contain olefinic double bonds or other centers of geometric asymmetry, and unless specified otherwise, it is intended that the compounds include both E and Z geometric isomers. Likewise, all tautomeric forms of carbonyl-containing compounds are also intended to be included.

It is to be understood that the compounds provided herein may contain chiral centers. Such chiral centers may be of either the (R) or (S) configuration, or may be a mixture thereof. Thus, the compounds provided herein may be enantiomerically pure, or may be stereoisomeric or diastereomeric mixtures. As such, one of skill in the art will recognize that administration of a compound in its (R) form is equivalent, for compounds that undergo epimerization in vivo, to administration of the compound in its (S) form.

Compounds described herein include, but are not limited to, those set forth above, as well as any of their isomers, such as diastereomers and enantiomers, as well as salts, esters, amides, thioesters, solvates, and polymorphs thereof, as well as racemic mixtures and pure isomers of the compounds set forth above.

### Methods of the Present Disclosure

In some aspects, the present disclosure provides a method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents or reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the conditioning regimen of the comparable method comprises administering to the patient busulfan (Bu), fludarabine (Flu), and rATG.

In some aspects, the present disclosure provides a method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents, or reduces the severity of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the conditioning regimen of the comparable method comprises administering to the patient busulfan (Bu), fludarabine (Flu), and rATG.

In some aspects, the present disclosure provides a method of preventing, or reducing the risk of, autoimmune cytopenia in a patient in need thereof, the method comprising: i) conditioning the patient with a conditioning regimen; and ii) administering to (e.g., infusing into) the patient a population of hematopoietic stem or progenitor cells (e.g., expanded cord blood (e.g., MGTA-456)).

In some aspects, the present disclosure provides a method of preparing a patient for hematopoietic stem or progenitor cell transplantation, the method comprising conditioning the patient with a conditioning regimen.

In some aspects, the present disclosure provides a method (e.g., of administering hematopoietic stem cell transplantation therapy to a patient in need thereof), the method comprising administering to (e.g., infusing into) the patient a population of hematopoietic stem or progenitor cells (e.g., expanded cord blood (e.g., MGTA-456)), wherein the patient has previously been conditioned with a conditioning regimen.

In some aspects, the present disclosure provides a method (e.g., of administering hematopoietic stem cell transplantation therapy to a patient in need thereof), the method comprising: a) conditioning the patient with a conditioning regimen; and b) administering to (e.g., infusing into) the patient a population of hematopoietic stem or progenitor cells (e.g., expanded cord blood (e.g., MGTA-456)).

In some aspects, the present disclosure provides conditioning regimens or agents useful in conjunction with the compositions and methods described herein include conditions that ablate patient bone marrow.

In some aspects, the present disclosure provides a population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the risk of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

In some aspects, the present disclosure provides a population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the severity of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

In some aspects, the present disclosure provides a combination of a conditioning regimen and a population of hematopoietic stem or progenitor cells for preventing, or reducing the risk of, autoimmune cytopenia in a patient in need thereof, wherein the patient is conditioned with the conditioning regimen prior to being administered with the population of hematopoietic stem or progenitor cells.

In some aspects, the present disclosure provides a combination of a conditioning regimen and an expanded cord blood for preventing, or reducing the risk of, autoimmune cytopenia in a patient in need thereof, wherein the patient is conditioned with the conditioning regimen prior to being administered with the expanded cord blood, and wherein the conditioning regimen does not comprise busulfan plus fludarabine (BuFlu).

In some aspects, the present disclosure provides a population of hematopoietic stem or progenitor cells for being administered to a patient, wherein the patient is conditioned with a conditioning regimen prior to the administration of the population of hematopoietic stem or progenitor cells.

In some aspects, the present disclosure provides a population of hematopoietic stem or progenitor cells for administering hematopoietic stem cell transplantation therapy to a patient in need thereof, wherein the is conditioned with a conditioning regimen prior to infusing into the patient the population of hematopoietic stem or progenitor cells.

In some aspects, the present disclosure provides a conditioning regimen (e.g., prophylactic agent) for preventing or reducing the risk of autoimmune cytopenia in a patient in need thereof, wherein the conditioning regimen (e.g., prophylactic agent) is administered to the patient prior to, during, or following ttransplanting the patient with expanded cord blood; and wherein the conditioning regimen (e.g., prophylactic agent) inhibits the production of antibodies in the patient.

In some aspects, the present disclosure provides a conditioning regimen for preparing a patient for hematopoietic stem or progenitor cell transplantation.

In some aspects, the present disclosure provides a population of hematopoietic stem or progenitor cells for administering hematopoietic stem or progenitor cell transplant therapy to a patient in need thereof, wherein the population of hematopoietic stem or progenitor cells that have been expanded ex vivo, wherein the population, prior to expansion, comprises no more than 1×10⁸ CD34+ cells.

In some aspects, the present disclosure provides a population of hematopoietic stem or progenitor cells for treating a stem cell disorder in a patient.

In some embodiments, the conditioning regimen comprise administering radiation and/or a chemical substance or prophylactic agent to the patient in order to ablate the bone marrow.

In some embodiments, the conditioning regimen is myeloablative.

In some embodiments, the conditioning regimen comprise a prophylactic agent.

In some embodiments, the patient is administered a prophylactic agent prior to, during, or following transplant with the expanded cord blood to prevent or reduce the risk of autoimmune cytopenia. The prophylactic agent is one that inhibits production of antibodies, for example an anti-CD20 antibody such as Rituxan (generic name: rituximab). In some embodiments, the rituximab is administered in combination with intravenous immunoglobulin (IVIG) so that the patient has some antibodies to ward off infection while the prophylactic agent is preventing the patient from producing the patient's own antibodies.

In some embodiments, the patient is conditioned using a conditioning regimen that is not busulfan plus fludarabine (BuFlu) (i.e., does not comprise busulfan and fludarabine). Without wishing to be bound by any theory, it is believed that BuFlu may not sufficiently ablate enough of the patient's B cells, so a more intense myeloablative conditioning regimen that substantially ablates the B cells may be needed to prevent or reduce the risk of autoimmune cytopenia. In some embodiments, the conditioning regimen may comprise busulfan plus cyclophosphamide (BuCy).

In some embodiments, upon transplantation, the risk of autoimmune cytopenia is prevented or reduced in the patient relative to a patient that is not conditioned with the conditioning regimen.

Conditioning agents useful in conjunction with the compositions and methods described herein may include antibodies and antigen-binding fragments thereof, such as those that bind one or more antigens on a B cell, and promote the death of the B cell. Such antibodies and antigen-binding fragments thereof may be conjugated to a toxin or may be administered alone.

Myeloablative conditioning agents useful in conjunction with the compositions and methods described herein include those that selectively target a marker and facilitate the intracellular delivery of an immunotoxin to one or more cells of the target tissue, for example, B cells in the bone marrow tissue of a subject. By selectively targeting cells expressing a selected marker, conditioning agents may be able to exert their cytotoxic effect on those targeted cells, while sparing, minimizing, and in certain instances eliminating, adverse effects on non-targeted cells and tissues.

In some aspects, the present disclosure relates to a method of preventing or reducing the risk of autoimmune cytopenia in a patient in need thereof, the method comprising: i) administering a prophylactic agent prior to, during, or following transplant with expanded cord blood; and ii) transplanting the patient with expanded cord blood; wherein the prophylactic agent inhibits the production of antibodies in the patient.

In some embodiments, the expanded cord blood has been expanded with an aryl hydrocarbon receptor antagonist.

In some embodiments, the prophylactic agent is an anti-CD20 antibody.

In some embodiments, the anti-CD20 antibody is rituximab.

In some embodiments, the prophylactic agent is administered in combination with intravenous immunoglobulin (IVIG).

In some aspects, the present disclosure relates to a method of preventing or reducing the risk of autoimmune cytopenia in a patient in need thereof, the method comprising: i) conditioning the patient with a conditioning regimen; and ii) transplanting the patient with expanded cord blood; wherein the conditioning regimen is not busulfan plus fludarabine (BuFlu).

In some embodiments, the expanded cord blood has been expanded with an aryl hydrocarbon receptor antagonist.

In some embodiments, the conditioning regimen comprises busulfan plus cyclophosphamide (BuCy).

In some embodiments, the conditioning regimen substantially ablates the patient's B-cells.

In some embodiments, the expanded cord blood is MGTA-456.

In some embodiments, the patient may be any age. In certain embodiments, the patient may be 17 years old or younger. In certain embodiments, the patient may be 15 years old or younger. In some embodiments, the patient may be 2 years old or younger. In some embodiments, the patient may be at least 6 months old. In some embodiments, the patient may be between 6 months old and 2 years old. In some embodiments, the patient may be aged between 0 months old and 72 months old, 0 months old and 60 months old, 0 months old and 48 months old, 0 months old and 36 months old, between 0 months old and 24 months old, between 0 months old and 12 months old, between 0 months old and 8 months old, between 0 months old and 6 months old, between 0 months old and 4 months old, between 1 month old and 72 months old, between 1 month old and 60 months old, between 1 month old and 48 months old, between 1 month old and 36 months old, between 1 month old and 24 months old, between 1 month old and 12 months old, between 1 month old and 6 months old, or between 1 month old and 4 months old.

In some embodiments, the patient has an inherited metabolic disorder.

In some embodiments, the inherited metabolic disorder is Hurler disease, metachromatic leukodystrophy, globoid cell leukodystrophy, or cerebral adrenoleukodystrophy.

In some aspects, the present disclosure relates to a method of administering hematopoietic stem or progenitor cell transplant therapy to a patient in need thereof, the method comprising: a) conditioning the patient with a conditioning regimen; and b) infusing into the patient a population of hematopoietic stem or progenitor cells.

In some aspects, the present disclosure relates to a method of preparing a patient for hematopoietic stem or progenitor cell transplantation, the method comprising conditioning the patient with a conditioning regimen.

In some aspects, the present disclosure relates to a method of administering hematopoietic stem cell transplantation therapy to a patient in need thereof, wherein the patient has previously been conditioned with a conditioning regimen, the method comprising infusing into the patient a population of hematopoietic stem or progenitor cells.

In some embodiments, the conditioning regimen comprises administering radiation and/or a prophylactic agent to the patient.

In some embodiments, the conditioning regimen is administered prior to, during, or following the infusion of a population of stem or progenitor cells.

In some embodiments, the conditioning regimen is administered prior to the infusing of a population of hematopoietic stem or progenitor cells.

In some embodiments, the prophylactic agent is an anti-CD20 antibody.

In some embodiments, the prophylactic agent is rituximab.

In some embodiments, the conditioning regimen comprises administering one or more alkylating agents.

In some embodiments, the conditioning regimen comprises administering one or more alkylating agents including, but not limited to, nitrogen mustards (e.g. Cyclophosphamide. Chlormethine, Uramustine, Melphalan, Chlorambucil, Infosfamide, Bendabustine), nitrosoureas (e.g., Carmustine, Lomustine, Streptozocin), alkyl sulfonates (e.g., Busulfan), and the like.

In some embodiments, the conditioning regimen comprises administering the alkylating agent Cyclophosphamide (Cy).

In some embodiments, the conditioning regimen comprises administering the alkylating agent Busulfan (Bu).

In some embodiments, the conditioning regimen comprises administering two alkylating agents.

In some embodiments, the conditioning regimen comprises administering two alkylating agents simultaneously, sequentially or in alteration.

In some embodiments, the conditioning regimen comprises administering two alkylating agents sequentially.

In some embodiments, the conditioning regimen comprises administering Cyclophosphamide and Busulfan (BuCy).

In some embodiments, the conditioning regimen comprises administering Cyclophosphamide and Busulfan simultaneously, sequentially, or in alteration.

In some embodiments, the conditioning regimen comprises administering Cyclophosphamide and Busulfan sequentially.

In some embodiments, the conditioning regimen comprises administering Busulfan prior to administering Cyclophosphamide.

In some embodiments, the conditioning regimen does not comprise administering a purine analog.

In some embodiments, the conditioning regimen does not comprise administering fludarabine (Flu).

In some embodiments, the conditioning regimen does not comprise administering Busulfan and fludarabine (BuFlu).

In some embodiments, the conditioning regimen does not comprise administering Busulfan and fludarabine (BuFlu) simultaneously, sequentially, or in alteration.

In some embodiments, the conditioning regimen comprises administering an anti-leukocyte globulin.

In some embodiments, the conditioning regimen comprises administering an anti-leukocyte globulin including, but not limited to, polyclonal and monoclonal anti-leukocyte globulins such as, for example, anti-lymphocyte globulin (ALG), anti-T lymphocyte globulin, anti-thymocyte globulin (ATG) and antibodies against well-defined T lymphocyte subsets. In some embodiments, the anti-leukocyte globulin may comprise an anti-CD52 antibody (e.g., alemtuzumab (Campath)).

In some embodiments, the conditioning regimen comprises administering an anti-thymocyte globulin (ATG). As used herein, anti-thymocyte globulin (ATG) refers to an infusion of antibodies (e.g., horse, rabbit, or pig-derived antibodies) against human T-cells, In some embodiments, the anti-thymocyte globulin (ATG) is one of two anti-thymocyte globulin (ATG) agents licensed for clinical use in United States including Thymoglobulin® (anti-thymocyte globulin (rabbit), rabbit ATG, rATG; Sanofi/Genzyme) and/or Atgam® (lymphocyte immune globulin, anti-thymocyte globulin [equine], equine ATG, eATG; Pfizer). In some embodiments, the anti-thymocyte globulin is porcine ATG (pATG).

In some embodiments, the conditioning regimen comprises administering anti-thymocyte globulin (equine) (i.e., Atgam).

In some embodiments, the conditioning regimen comprises administering anti-thymocyte globulin (rabbit) (i.e., Thymoglobulin).

In some embodiments, the conditioning regimen comprises administering an anti-thymocyte globulin (ATG) and one or more alkylating agents.

In some embodiments, the conditioning regimen comprises administering an anti-thymocyte globulin (ATG) and one or more alkylating agents simultaneously, sequentially or in alteration.

In some embodiments, the conditioning regimen comprises administering an anti-thymocyte globulin (ATG) and two alkylating agents.

In some embodiments, the conditioning regimen comprises administering an anti-thymocyte globulin (ATG) and two alkylating agents simultaneously, sequentially or in alteration.

In some embodiments, the conditioning regimen comprises administering a first alkylating agent prior to an anti-thymocyte globulin (ATG) and administering a second alkylating agent simultaneously with an anti-thymocyte globulin (ATG).

In some embodiments, the conditioning regimen comprises administering anti-thymocyte globulin (rabbit) (i.e., Thymoglobulin, ATG) and Cyclophosphamide (Cy).

In some embodiments, the conditioning regimen comprises administering anti-thymocyte globulin (rabbit) (i.e., Thymoglobulin, ATG) and Cyclophosphamide (Cy) simultaneously, sequentially or in alteration.

In some embodiments, the conditioning regimen comprises administering anti-thymocyte globulin (rabbit) (i.e., Thymoglobulin, ATG) and Cyclophosphamide (Cy) simultaneously.

In some embodiments, the conditioning regimen comprises administering anti-thymocyte globulin (rabbit) (i.e., Thymoglobulin, ATG) and Busulfan (Bu).

In some embodiments, the conditioning regimen comprises administering anti-thymocyte globulin (rabbit) (i.e., Thymoglobulin, ATG) and Busulfan (Bu) sequentially.

In some embodiments, the conditioning regimen comprises administering Busulfan (Bu) prior to administering anti-thymocyte globulin (rabbit) (i.e., Thymoglobulin, ATG).

In some embodiments, the conditioning regimen comprises administering anti-thymocyte globulin (rabbit) (i.e., Thymoglobulin, ATG), Busulfan (Bu), and Cyclophosphamide (Cy).

In some embodiments, the conditioning regimen comprises administering anti-thymocyte globulin (rabbit), Busulfan, and Cyclophosphamide (BuCyATG) simultaneously, sequentially or in alteration.

In some embodiments, the conditioning regimen comprises administering Busulfan (Bu) prior to administering anti-thymocyte globulin (rabbit) (i.e., Thymoglobulin, ATG) and administering Cyclophosphamide (Cy) simultaneously with administering anti-thymocyte globulin (rabbit) (i.e., Thymoglobulin, ATG).

In some aspects, the present disclosure relates to a method of administering hematopoietic stem or progenitor cell transplant therapy to a patient in need thereof, the method comprising: a) conditioning the patient with a conditioning regimen, the conditioning regimen comprising a1) administering busulfan (Bu); a2) administering cyclophosphamide (Cy); and a3) administering anti-thymocyte globulin (rabbit)(ATG); and b) infusing into the patient a population of hematopoietic stem or progenitor cells.

In some aspects, the present disclosure relates to a method of administering hematopoietic stem or progenitor cell transplant therapy to a patient in need thereof, the method comprising: a) conditioning the patient with a conditioning regimen, the conditioning regimen comprising a1) administering busulfan (Bu) prior to administering cyclophosphamide and prior to administering anti-thymocyte globulin (rabbit) (ATG); a2) administering cyclophosphamide (Cy) after administering busulfan (Bu) and simultaneously with administering anti-thymocyte globulin (rabbit) (ATG); and a3) administering anti-thymocyte globulin (rabbit) (ATG) after administering busulfan (Bu) and simultaneously with administering cyclophosphamide (Cy); and b) infusing into the patient a population of hematopoietic stem or progenitor cells.

In some embodiments, the busulfan (Bu) is administered intravenously.

In some embodiments, the busulfan (Bu) is administered at a dose wherein the plasma exposure as measured by cumulative AUC is maintained within a range of 74-82 mg*hr/L.

In some embodiments, the busulfan (Bu) is administered at a dose wherein the plasma exposure as measured by cumulative AUC is maintained within at about 78 mg*hr/L.

In some embodiments, the busulfan (Bu) is administered at a dose wherein the plasma exposure as measured by steady state concentration (Cₛₛ) is maintained within a range of 770-850 ng/mL.

In some embodiments, the busulfan (Bu) is administered at a dose wherein the plasma exposure as measured by steady state concentration (Cₛₛ) is maintained at about 810 ng/mL.

In some embodiments, the busulfan (Bu) is administered in a total of 4 doses.

In some embodiments, the busulfan (Bu) is administered in a total of 4 doses once daily.

In some embodiments, the busulfan (Bu) is administered in a total of 4 doses once daily over a time period of about 3 hours per dose.

In some embodiments, the busulfan (Bu) is administered in a total of 4 doses once daily with an initial dose in a range of about 80 mg/m2 to about 120 mg/m2.

In some embodiments, the busulfan (Bu) is administered in a total of 16 doses.

In some embodiments, the busulfan (Bu) is administered in a total of 16 doses with one dose given every 6 hours.

In some embodiments, the busulfan (Bu) is administered in a total of 16 doses with one dose given every 6 hours over a time period of about 2 hours per dose.

In some embodiments, the busulfan (Bu) is administered in a total of 16 doses every 6 hours with a dose of about 0.6 to about 1.2 mg/kg. In some embodiments, the busulfan (Bu) is administered in a total of 16 doses about every 6 hours with an initial dose of about 1 mg/kg.

In some embodiments, the busulfan (Bu) is administered for about 4 consecutive days.

In some embodiments, the busulfan (Bu) is administered for 4 consecutive days at days −9 to −6 prior to infusing into the patient a population of hematopoietic stem or progenitor cells.

In some embodiments, the cyclophosphamide (Cy) is administered intravenously.

In some embodiments, the cyclophosphamide (Cy) is administered at a dosage of about 50 to about 60 mg/kg/day. In some embodiments, the cyclophosphamide (Cy) is administered at a dosage of about 50 mg/kg/day.

In some embodiments, the daily dosage of about 50 mg/kg/day of the cyclophosphamide (Cy) is administered over a time period of about 1 hour per dose.

In some embodiments, the cyclophosphamide (Cy) is administered for about 4 consecutive days.

In some embodiments, the cyclophosphamide (Cy) is administered for 4 consecutive days at days −5 to −2 prior to infusing into the patient a population of hematopoietic stem or progenitor cells.

In some embodiments, the first dose of the cyclophosphamide (Cy) is administered at least 24 hours after the last dose of busulfan (Bu).

In some embodiments, the anti-thymocyte globulin (rabbit) (Thymoglobulin, ATG) is administered intravenously.

In some embodiments, the anti-thymocyte globulin (rabbit) (Thymoglobulin, ATG) is administered at a dosage of about 1.5 to about 5 mg/kg/day. In some embodiments, the anti-thymocyte globulin (rabbit) (Thymoglobulin, ATG) is administered at a dosage of about 2.5 mg/kg/day.

In some embodiments, the the anti-thymocyte globulin (rabbit) (Thymoglobulin, ATG) is administered at a total dosage of about 7.5 to about 10 mg/kg. In some embodiments, the anti-thymocyte globulin (rabbit) (Thymoglobulin, ATG) is administered at a total dosage of about 10 mg/kg.

In some embodiments, the daily dosage of about 2.5 mg/kg/day of the anti-thymocyte globulin (rabbit) (Thymoglobulin, ATG) is administered over a time period of about 2 hours to about 10 hours.

In some embodiments, the daily dosage of about 2.5 mg/kg/day of the anti-thymocyte globulin (rabbit) (Thymoglobulin, ATG) is administered over a time period of about 6 hours per dose.

In some embodiments, the anti-thymocyte globulin (rabbit) (Thymoglobulin, ATG) is administered for about 4 consecutive days.

In some embodiments, the anti-thymocyte globulin (rabbit) (Thymoglobulin, ATG) is administered for 4 consecutive days at days −5 to −2 prior to infusing into the patient a population of hematopoietic stem or progenitor cells.

In some aspects, the present disclosure relates to a method of administering hematopoietic stem or progenitor cell transplant therapy to a patient in need thereof, the method comprising: a) conditioning the patient with a conditioning regimen, the conditioning regimen comprising a1) administering busulfan (Bu) at days −9 to −6 prior to infusing into the patient a population of hematopoietic stem or progenitor cells; a2) administering cyclophosphamide (Cy) at days −5 to −2 prior to infusing into the patient a population of hematopoietic stem or progenitor cells; and a3) administering anti-thymocyte globulin (rabbit)(ATG) at days −5 to −2 prior to infusing into the patient a population of hematopoietic stem or progenitor cells; and b) infusing into the patient a population of hematopoietic stem or progenitor cells at day 0.

In some aspects, the present disclosure relates to a method of administering hematopoietic stem or progenitor cell transplant therapy to a patient in need thereof, the method comprising: a) conditioning the patient with a conditioning regimen, the conditioning regimen comprising a1) administering busulfan (Bu) at a dose wherein the plasma exposure as measured by cumulative AUC is maintained within a range of 74-82 mg*hr/L for 4 consecutive days at days −9 to −6 prior to infusing into the patient a population of hematopoietic stem or progenitor cells; a2) administering cyclophosphamide (Cy) at a dose of 50 mg/kg/day for 4 consecutive days at days −5 to −2 prior to infusing into the patient a population of hematopoietic stem or progenitor cells; and a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and b) infusing into the patient a population of hematopoietic stem or progenitor cells at day 0.

In some aspects, the present disclosure relates to a method of administering hematopoietic stem or progenitor cell transplant therapy to a patient in need thereof, the method comprising: a) conditioning the patient with a conditioning regimen, the conditioning regimen comprising a1) administering busulfan (Bu) at a dose wherein the plasma exposure as measured by steady state concentration (Css) is maintained within a range of 770-850 ng/mL for 4 consecutive days at days −9 to −6 prior to infusing into the patient a population of hematopoietic stem or progenitor cells; a2) administering cyclophosphamide (Cy) at a dose of 50 mg/kg/day for 4 consecutive days at days −5 to −2 prior to infusing into the patient a population of hematopoietic stem or progenitor cells; and a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and b) infusing into the patient a population of hematopoietic stem or progenitor cells at day 0.

In some embodiments, upon transplantation, the risk of autoimmune cytopenia is prevented or reduced in the patient relative to a patient that is administered a conditioning regimen comprising busulfan and fludarabine (BuFlu) prior to transplantation. For example, in some embodiments, upon transplantation, the risk of autoimmune cytopenia is prevented or reduced in the patient relative to a patient that is administered a conditioning regimen comprising busulfan, fludarabine, and ATG (e.g., rATG) prior to transplantation.

In some embodiments, upon transplantation, the risk of autoimmune cytopenia is prevented or reduced in a patient administered a conditioning regimen comprising busulfan and cyclophosphamide relative to a patient that is administered a conditioning regimen comprising busulfan and fludarabine (BuFlu). For example, in some embodiments, upon transplantation, the risk of autoimmune cytopenia is prevented or reduced in a patient administered a conditioning regimen comprising busulfan and cyclophosphamide relative to a patient that is administered a conditioning regimen comprising busulfan, fludarabine, and ATG (e.g., rATG).

In some embodiments, upon transplantation, the risk of autoimmune cytopenia is prevented or reduced in a patient administered a conditioning regimen comprising busulfan, cyclophosphamide, and rabbit ATG relative to a patient that is administered a conditioning regimen comprising busulfan and fludarabine (BuFlu). In some embodiments, upon transplantation, the risk of autoimmune cytopenia is prevented or reduced in a patient administered a conditioning regimen comprising busulfan, cyclophosphamide, and rabbit ATG relative to a patient that is administered a conditioning regimen comprising busulfan, fludarabine, and ATG (e.g., rATG). In some embodiments, upon transplantation, the risk of autoimmune cytopenia is prevented or reduced in a patient administered a conditioning regimen comprising busulfan, cyclophosphamide, and equine ATG relative to a patient that is administered a conditioning regimen comprising busulfan and fludarabine (BuFlu). In some embodiments, upon transplantation, the risk of autoimmune cytopenia is prevented or reduced in a patient administered a conditioning regimen comprising busulfan, cyclophosphamide, and equine ATG relative to a patient that is administered a conditioning regimen comprising busulfan, fludarabine, and ATG (e.g., rATG).

In some embodiments, upon transplantation, the risk of autoimmune cytopenia is prevented or reduced in a patient administered a conditioning regimen comprising busulfan, cyclophosphamide, and rabbit ATG relative to a patient that is administered a conditioning regimen comprising busulfan, cyclophosphamide, and equine ATG. In some embodiments, upon transplantation, the risk of autoimmune cytopenia is prevented or reduced in a patient administered a conditioning regimen comprising busulfan, cyclophosphamide, and equine ATG relative to a patient that is administered a conditioning regimen comprising busulfan, cyclophosphamide, and rabbit ATG.

In some embodiments, upon transplantation, the risk of autoimmune cytopenia with an onset of at least 2 days following the infusing into the patient a population of hematopoietic stem or progenitor cells, at least 5 days, at least 10 days, at least 20 days, at least 25 days, at least 50 days, at least 75 days, at least 100 days, at least 125 days, at least 150 days, at least 175 days, at least 200 days, at least 250 days, at least 300 days, or at least 350 days following the infusing into the patient a population of hematopoietic stem or progenitor cells is prevented or reduced in the patient relative to a patient that is administered a conditioning regimen comprising busulfan and fludarabine (BuFlu) prior to transplantation.

In some embodiments, the patient has an inherited metabolic disorder.

In some embodiments, the inherited metabolic disorder is Hurler disease, metachromatic leukodystrophy, globoid cell leukodystrophy, or cerebral adrenoleukodystrophy.

In some embodiments, the hematopoietic stem or progenitor cells, or progeny thereof, maintain hematopoietic stem cell functional potential after 2 or more days (e.g., for about 2 days, 3 days, 4 days, 5 days, 6 days, 7 days, 8 days, 9 days, 10 days, or more) following infusion of the hematopoietic stem or progenitor cells into the patient.

In some embodiments, the hematopoietic stem or progenitor cells, or progeny thereof, localize to hematopoietic tissue and/or reestablish hematopoiesis following infusion of the hematopoietic stem or progenitor cells into the patient.

In some embodiments, upon infusion into the patient, the hematopoietic stem or progenitor cells give rise to recovery of a population of cells selected from the group consisting of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

In some embodiments, the conditioning regimen comprises administering radiation and/or a prophylactic agent to the patient.

In some embodiments, the conditioning regimen is administered prior to, during, or following the infusion of a population of stem or progenitor cells.

In some embodiments, the prophylactic agent is an anti-CD20 antibody.

In some embodiments, the prophylactic agent is rituximab.

In some embodiments, the prophylactic agent is administered in combination with intravenous immunoglobulin (IVIG).

In some embodiments, the conditioning regimen does not comprise busulfan plus fludarabine (BuFlu).

In some embodiments, the conditioning regimen comprises busulfan plus cyclophosphamide (BuCy).

In some embodiments, the conditioning regimen substantially ablates the B cells of the patient.

In some embodiments, the conditioning regimen ablates at least 50% of the B cells of the patient, at least 60% of the B cells of the patient, at least 70% of the B cells of the patient, at least 75% of the B cells of the patient, at least 80% of the B cells of the patient, at least 85% of the B cells of the patient, at least 90% of the B cells of the patient, at least 95% of the B cells of the patient, or at least 98% of the B cells of the patient.

In some embodiments, the patient is 2 years old or younger.

In some embodiments, the method further comprises administering a prophylactic agent against seizures prior to, during, or following the administering of busulfan (Bu).

In some embodiments, the prophylactic agent against seizures is levetiracetam (Keppra).

In some embodiments, the first dose of the prophylactic agent against seizures is administered at least about 12-24 hours prior to the first dose of busulfan (Bu) is administered and the last dose of the prophylactic agent against seizures is administered at least about 24 hours after the last dose of busulfan (Bu) is administered.

In some embodiments, the method further comprises administering a chemotherapy adjuvant prior to, during, or following the administering of cyclophosphamide.

In some embodiments, the chemotherapy adjuvant is mesna (Mesnex).

In some embodiments, the chemotherapy adjuvant is administered during the administering of cyclophosphamide.

In some embodiments, the chemotherapy adjuvant is administered during the administering of cyclophosphamide at days −5 to −2 prior to infusing into the patient a population of hematopoietic stem or progenitor cells.

In some embodiments, the method further comprises administering an immunosuppression regimen to the patient.

In some embodiments, the immunosuppression regimen comprises administering at least one immunosuppressant agent.

In some embodiments, the immunosuppressant agent is administered prior to, during, or following the conditioning regimen.

In some embodiments, the immunosuppressant agent is administered during and following the conditioning regimen.

In some embodiments, the immunosuppressant agent is mycophenolate mofetil (MMF, CellCept), cyclosporine A (CsA), and/or salts or prodrugs thereof.

In some embodiments, the immunosuppressant agent is mycophenolate mofetil (MMF).

In some embodiments, the immunosuppressant agent is cyclosporine A (CsA).

In some embodiments, the immunosuppression regimen comprises administering mycophenolate mofetil (MMF) and cyclosporine A (CsA).

In some embodiments, the immunosuppression regimen comprises administering mycophenolate mofetil (MMF) and cyclosporine A (CsA) starting at day −3 prior to infusing into the patient a population of hematopoietic stem or progenitor cells.

In some embodiments, cyclosporine A (CsA) is administered for at a dose wherein the serum trough level is maintained within a range of about 200-400 ng/mL.

In some embodiments, cyclosporine A (CsA) is administered for at least 200 days following the infusion of a population of hematopoietic stem or progenitor cells.

In some embodiments, mycophenolate mofetil (MMF) is administered intravenously or orally.

In some embodiments, mycophenolate mofetil (MMF) is administered for about three times daily for at least 40 days following the infusion of a population of hematopoietic stem or progenitor cells.

In some embodiments, mycophenolate mofetil (MMF) is administered at a dose of about 300 mg/kg/day to about 3000 mg/kg/day.

In some embodiments, the method further comprises administering granulocyte colony-stimulating factor (G-CSF) prior to, during, or following the infusion of a population of hematopoietic stem or progenitor cells.

In some embodiments, the granulocyte colony-stimulating factor (G-CSF) is administered following the infusion of a population of hematopoietic stem or progenitor cells.

In some embodiments, the granulocyte colony-stimulating factor (G-CSF) is administered starting at day +1 following the infusion of a population of hematopoietic stem or progenitor cells.

In some embodiments, the granulocyte colony-stimulating factor (G-CSF) is administered starting at day +1 following the infusion of a population of hematopoietic stem or progenitor cells until an absolute neutrophil count (ANC) is greater than or equal to about 2,500/μL for at least 2 consecutive days.

In some aspects, the present disclosure provides a method (e.g., of administering hematopoietic stem or progenitor cell transplant therapy to a patient in need thereof), the method comprising: (a) expanding, ex vivo, a population of hematopoietic stem or progenitor cells (e.g., CD34+ cells) comprising no more than 1×10⁸ CD34+ cells; and (b) infusing into the patient the expanded population of hematopoietic stem or progenitor cells, or progeny thereof.

In some aspects, the present disclosure provides a method (e.g., of administering hematopoietic stem or progenitor cell transplant therapy to a patient in need thereof), the method comprising infusing into the patient a population of hematopoietic stem or progenitor cells that have been expanded ex vivo, wherein the population, prior to expansion, comprises no more than 1×10⁸ CD34+ cells.

In some embodiments, the population, prior to expansion, comprises no more than 9×10⁷ CD34+ cells.

In some embodiments, the population, prior to expansion, comprises no more than 8×10⁷ CD34+ cells.

In some embodiments, the population, prior to expansion, comprises no more than 7×10⁷ CD34+ cells.

In some embodiments, the population, prior to expansion, comprises no more than 6×10⁷ CD34+ cells.

In some embodiments, the population, prior to expansion, comprises no more than 5×10⁷ CD34+ cells.

In some embodiments, the population, prior to expansion, comprises no more than 9×10⁶ CD34+ cells.

In some embodiments, the population, prior to expansion, comprises no more than 8×10⁶ CD34+ cells.

In some embodiments, the population, prior to expansion, comprises no more than 7×10⁸ CD34+ cells.

In some embodiments, the population, prior to expansion, comprises no more than 6×10⁸ CD34+ cells.

In some embodiments, the population, prior to expansion, comprises no more than 5×10⁸ CD34+ cells.

In some embodiments, the population, prior to expansion, comprises no more than 1×10⁸ CD34+ cells.

In some embodiments, the step of expanding comprises contacting the population of hematopoietic stem or progenitor cells (e.g., CD34+ cells) with an aryl hydrocarbon receptor antagonist (e.g, SR-1, compound 2, a compound represented by formula (IV), or a compound represented by formula (V)).

In some embodiments, prior to infusion into the patient, the hematopoietic stem or progenitor cells are mobilized and isolated from a donor.

In some embodiments, the donor is a human.

In some embodiments, the hematopoietic stem or progenitor cells are mobilized by contacting the hematopoietic stem or progenitor cells with a mobilizing amount of a CXCR4 antagonist and/or a CXCR2 agonist.

In some embodiments, the CXCR4 antagonist is plerixafor or BL-8040.

In some embodiments, the CXCR2 agonist is Gro-β, Gro-β T, or a variant thereof.

In some aspects, the present disclosure provides a method of treating or preventing a disorder (e.g., a stem cell disorder) in a patient in need thereof, the method comprising administering to (e.g., infusing into) the patient a population of hematopoietic stem or progenitor cells (e.g., expanded cord blood (e.g., MGTA-458)), wherein the patient has previously been conditioned with a conditioning regimen.

In some aspects, the present disclosure provides a method of treating or preventing a disorder (e.g., a stem cell disorder) in a patient in need thereof, the method comprising: a) conditioning the patient with a conditioning regimen; and b) administering to (e.g., infusing into) the patient a population of hematopoietic stem or progenitor cells (e.g., expanded cord blood (e.g., MGTA-456)).

In some aspects, the present disclosure provides a method of treating a stem cell disorder in a patient (e.g., a human patient), the method comprising administering hematopoietic stem or progenitor cell transplant therapy to the patient in accordance with the method of any of the foregoing aspects or embodiments.

In some embodiments, the stem cell disorder is a hemoglobinopathy disorder. The hemoglobinopathy disorder may be, for example, sickle cell anemia, thalassemia, Fanconi anemia, aplastic anemia, or Wiskott-Aldrich syndrome.

In some embodiments, the stem cell disorder is a myelodysplastic disorder. In some embodiments, the stem cell disorder is an immunodeficiency disorder, such as a congenital immunodeficiency or an acquired immunodeficiency, such as human immunodeficiency virus or acquired immune deficiency syndrome.

In some embodiments, the stem cell disorder is a metabolic disorder, such as glycogen storage diseases, mucopolysaccharidoses, Gaucher disease, Hurler disease, sphingolipidoses, metachromatic leukodystrophy (MLD), globoid cell leukodystrophy (GLD, also referred to as Krabbe disease), or cerebral adrenoleukodystrophy (cALD).

In some embodiments, the stem cell disorder is an inherited metabolic disorder. Non-limiting examples of inherited metabolic disorders include Hurler disease, metachromatic leukodystrophy (MLD), globoid cell leukodystrophy (GLD, also referred to as Krabbe disease), and cerebral adrenoleukodystrophy (cALD).

In some embodiments, the stem cell disorder is cancer, such as leukemia, lymphoma, multiple myeloma, or neuroblastoma. The cancer may be, for instance, a hematological cancer. In some embodiments, the cancer is myeloid leukemia, acute lymphoid leukemia, chronic myeloid leukemia, chronic lymphoid leukemia, multiple myeloma, diffuse large B-cell lymphoma, or non-Hodgkin's lymphoma.

In some embodiments, the stem cell disorder is adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, or juvenile rheumatoid arthritis.

In some embodiments, the stem cell disorder is an autoimmune disorder, such as multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitists, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis. Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pemicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis.

In some embodiments, the hematopoietic stem cells are autologous with respect to the patient. For instance, autologous hematopoietic stem cells can be removed from a donor and the cells can subsequently be administered to (e.g., infused into) the patient so as to repopulate one or more cell types of the hematopoietic lineage.

In some embodiments, the hematopoietic stem cells are allogeneic with respect to the patient. For instance, allogeneic hematopoietic stem cells can be removed from a donor, such as donor that is HLA-matched with respect to the patient, for instance, a closely related family member of the patient. In some embodiments, the allogenic hematopoietic stem cells are HLA-mismatched with respect to the patient. Following withdrawal of the allogeneic hematopoietic stem cells from a donor, the cells can subsequently be administered to (e.g., infused into) the patient so as to repopulate one or more cell types of the hematopoietic lineage.

In some embodiments, the hematopoietic stem or progenitor cells, or progeny thereof, maintain hematopoietic stem cell functional potential after two or more days following infusion of the hematopoietic stem or progenitor cells into the patient. In some embodiments, the hematopoietic stem or progenitor cells, or progeny thereof, localize to hematopoietic tissue and/or reestablish hematopoiesis following infusion of the hematopoietic stem or progenitor cells into the patient. For instance, upon infusion into the patient, the hematopoietic stem or progenitor cells may give rise to recovery of a population of cells selected from the group consisting of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

### Stem Cells

In some embodiments, the stem cells of which the population is modified (e.g., expanded) with the compositions and methods described are capable of being expanded upon contacting the aryl hydrocarbon receptor antagonist. In some embodiments, the stem cells are genetically modified stem cells. In some embodiments, the stem cells are not genetically modified stem cells.

In some embodiments, the stem cells are embryonic stem cells or adult stem cells. In some embodiments, the stem cells are totipotentent stem cells, pluripotent stem cells, multipoteltent stem cells, oligopotent stem cells, or unipotent stem cells. In some embodiments, the stem cells are tissue-specific stem cells.

In some embodiments, the stem cells are hematopoietic stem cells, intestinal stem cells, osteoblastic stem cells, mesenchymal stem cells (i.e., lung mesenchymal stem cells, bone marrow-derived mesenchymal stromal cells, or bone marrow stromal cells), neural stem cells (i.e., neuronal dopaminergic stem cells or motor-neuronal stem cells), epithelial stem cells (i.e., lung epithelial stem cells, breast epithelial stem cells, vascular epithelial stem cells, or intestinal epithelial stem cells), cardiac myocyte progenitor stem cells, skin stem cells (i.e., epidermal stem cells or follicular stem cells (hair follicle stem cells)), skeletal muscle stem cells, adipose stem cells, liver stem cells, induced pluripotent stem cells, umbilical cord stem cells, amniotic fluid stem cells, limbal stem cells, dental pulp stem cells, placental stem cells, myoblasts, endothelial progenitor cells, exfoliated teeth derived stem cells, or hair follicle stem cells.

In some embodiments, the stem cells are hematopoietic stem cells.

In some embodiments, the stem cells are primary stem cells. For example, the stem cells are obtained from bone marrow, adipose tissue, or blood. In some embodiments, the the stem cells are cultured stem cells.

In some embodiments, the stem cells are CD34+ cells. In some embodiments, the stem cells are CD90+ cells. In some embodiments, the stem cells are CD45RA− cells. In some embodiments, the stem cells are CD34+CD90+ cells. In some embodiments, the stem cells are CD34+CD45RA-cells. In some embodiments, the stem cells are CD90+CD45RA− cells. In some embodiments, the stem cells are CD34+CD90+CD45RA− cells.

In some embodiments, the hematopoietic stem cells are extracted from the bone marrow, mobilized into the peripheral blood and then collected by apheresis, or isolated from umbilical cord blood units.

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoietic stem cells. In some embodiments, the hematopoietic stem cells are CD34+CD90+CD45RA− hematopoietic stem cells.

In some embodiments, the method reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu). For example, in some embodiments, the method reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu), fludarabine (Flu), and ATG (e.g., rATG).

In some embodiments, the method reduces the risk of autoimmune cytopenia in the patient by about 1% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more, as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the method prevents the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the method prevents autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the method reduces the severity of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, the method reduces the severity of autoimmune cytopenia in the patient by about 1% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more, as compared to a comparable method in which the patient is conditioned prior to receiving the population of expanded hematopoietic stem or progenitor cells by a conditioning regimen comprising administering to the patient busulfan (Bu) and fludarabine (Flu).

In some embodiments, in the comparable method, the patient is conditioned prior to receiving the population of expanded hematopoietic stem or progenitor cells by a conditioning regimen comprising administering to the patient busulfan (Bu), fludarabine (Flu), and ATG (e.g., rATG).

In some embodiments, in the comparable method, the patient is conditioned prior to receiving the population of expanded hematopoietic stem or progenitor cells by a conditioning regimen as described in Example 2.

In some embodiments, the comparable method is substantially the same as the method other than that the patient is conditioned prior to receiving the population of expanded hematopoietic stem or progenitor cells by a conditioning regimen comprising administering to the patient busulfan (Bu) and fludarabine (Flu).

In some embodiments, the comparable method is substantially the same as the method other than that the patient is conditioned prior to receiving the population of expanded hematopoietic stem or progenitor cells by a conditioning regimen comprising administering to the patient busulfan (Bu), fludarabine (Flu), and ATG (e.g., rATG).

### Gene-Modified Hematopoietic Stem and Progenitor Cells

Hematopoietic stem and progenitor cells for use in conjunction with the compositions and methods described herein include those that have been genetically modified, such as those that have been altered so as to express a therapeutic transgene. Compositions and methods for the genetic modification of hematopoietic stem and progenitor cells are described in the sections that follow.

The compositions and methods described herein provide strategies for disrupting a gene of interest and for promoting the expression of target genes in populations of hematopoietic stem and progenitor cells, as well as for expanding these cells. For instance, a population of hematopoietic stem cells may be expanded according to the methods described herein and may be genetically modified, e.g., so as to exhibit an altered gene expression pattern. Alternatively, a population of cells may be enriched with hematopoietic stem cells, or a population of hematopoietic stem cells may be maintained in a multi-potent state, and the cells may further be modified using established genome editing techniques known in the art. For instance, one may use a genome editing procedure to promote the expression of an exogenous gene or inhibit the expression of an endogenous gene within a hematopoietic stem cell. Populations of hematopoietic stem cells may be expanded, enriched, or maintained in a multi-potent state according to the methods described herein and subsequently genetically modified so as to express a desired target gene, or populations of these cells may be genetically modified first and then expanded, enriched, or maintained in a multi-potent state.

In some embodiments, the populations (e.g., plurality) of hematopoietic stem cells are expanded, enriched, or maintained in a multi-potent state according to the methods described herein by being contacted with an aryl hydrocarbon receptor antagonist as described herein and subsequently genetically modified so as to express a desired target gene and substantially maintain the engraftable properties of the hematopoietic stem cells cells. In some embodiments, the populations (e.g., plurality) of hematopoietic stem cells are expanded, enriched, or maintained in a multi-potent state according to the methods described herein by being contacted with an aryl hydrocarbon receptor antagonist as described herein and subjected to conditions during a period of time sufficient to induce cell cycling, and subsequently genetically modified so as to express a desired target gene and substantially maintain the engraftable properties of the hematopoietic stem cells cells. In some embodiments, the conditions sufficient to induce cell cycling may comprise contacting the hematopoietic stem cells with one or more cytokines in amounts sufficient to induce cell cycling. Non-limiting examples of cytokines include SCF, IL6, TPO, FLT3L, and combinations thereof. Other agents or methods may also be used to induce cell cycling.

In some embodiments, the period of time sufficient to induce cell cycling may be at least about 1 day, at least about 2 days, at least about 3 days, at least about 4 days, or at least about 5 days. In some embodiments, the period of time sufficient to induce cell cycling is about 1 to about 5 days, about 1 to about 4 days, about 2 to about 4 days, about 1 to about 3 days, or about 2 to about 3 days. In some embodiments, the period of time sufficient to induce cell cycling may vary depending on the lineage of the cells.

In some embodiments, contacting the hematopoietic stem cells with an aryl hydrocarbon receptor antagonist does not affect cell cycling. Advantageously, actively cycling cells may be more easily genetically modified so as to express a desired target gene than a non-cycling cell. Additionally, in some embodiments, contacting the hematopoietic stem cells with an aryl hydrocarbon receptor antagonist does not prevent stem cells from entering the cell cycle, and allows the stem cells to remain as stem cells (e.g., including dividing so as to multiply in number without substantially differentiating), delaying differentiation and prolonging engraftment potential relative to cells (e.g., hematopoietic stem cells) not contacted with an aryl hydrocarbon receptor antagonist.

In some embodiments, the populations (e.g., plurality) of hematopoietic stem cells are expanded, enriched, or maintained in a multi-potent state according to the methods described herein by being contacted with an aryl hydrocarbon receptor antagonist as described herein during at least a period of time sufficient to induce cell cycling and subsequently genetically modified so as to express a desired target gene resulting in improved genetic modification relative to a comparable method wherein the populations (e.g., plurality) of hematopoietic stem cells are not contacted with an aryl hydrocarbon receptor antagonist as described herein during a period of time sufficient to induce cell cycling prior to being subsequently genetically modified.

In some embodiments, the populations of hematopoietic stem cells are expanded, enriched, or maintained in a multi-potent state according to the methods described herein by being contacted with an aryl hydrocarbon receptor antagonist as described herein during a period of time sufficient to induce cell cycling and subsequently genetically modified so as to express a desired target gene resulting in improved engraftment potential relative to a comparable method wherein the the populations of hematopoietic stem cells are not contacted with an aryl hydrocarbon receptor antagonist as described herein during a period of time sufficient to induce cell cycling prior to being subsequently genetically modified.

In some embodiments, hematopoietic stem cells are expanded, enriched, or maintained in a multi-potent state according to the methods described herein by being contacted with an aryl hydrocarbon receptor antagonist as described herein during a period of time sufficient to induce cell cycling in substantially all of the hematopoietic stem cells.

In some embodiments, the populations (e.g., plurality) of hematopoietic stem cells are expanded subsequently to being genetically modified. For example, the hematopoietic stem cells may be expanded in the presence of an aryl hydrocarbon receptor antagonist subsequently to being genetically modified. Expansion of the genetically modified hematopoietic stem cells may be performed, for example, to increase the number of engraftable genetically modified cells in a hematopoietic stem cell graft.

A wide array of methods has been established for the incorporation of target genes into the genome of a cell (e.g., a mammalian cell, such as a murine or human cell) so as to facilitate the expression of such genes.

### Polynucleotides Encoding Target Genes

One example of a platform that can be used to facilitate the expression of a target gene in a hematopoietic stem cell is by the integration of the polynucleotide encoding a target gene into the nuclear genome of the cell. A variety of techniques have been developed for the introduction of exogenous genes into a eukaryotic genome. One such technique involves the insertion of a target gene into a vector, such as a viral vector. Vectors for use with the compositions and methods described herein can be introduced into a cell by a variety of methods, including transformation, transfection, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition. Cold Spring Harbor University Press, New York (2014); and Ausubel, et al., Current Protocols in Molecular Biology, John Wiley & Sons, New York (2015), the disclosures of each of which are incorporated herein by reference.

Exogenous genes can also be introduced into a mammalian cell through the use of a vector containing the gene of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Viral vectors containing the VSV-G protein are described in further detail, e.g., in U.S. Pat. No. 5,512,421; and in U.S. Pat. No. 5,670,354, the disclosures of each of which are incorporated by reference herein.

Recognition and binding of the polynucleotide encoding a target gene by mammalian RNA polymerase is an important molecular event for gene expression to occur. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Alternatively, promoters derived from viral genomes can be used for the stable expression of target genes in mammalian cells. Examples of functional viral promoters that can be used to promote mammalian expression of these enzymes include adenovirus late promoter, vaccinia virus 7.5K promoter, SV40 promoter, cytomegalovirus promoter, mouse mammary tumor virus (MMTV) promoter. LTR promoter of HIV, promoter of moloney virus, Epstein barr virus (EBV) promoter, Rous sarcoma virus (RSV) promoter, and the cytomegalovirus (CMV) promoter. Additional viral promoters include the SV40 late promoter from simian virus 40, the Baculovirus polyhedron enhancer/promoter element, Herpes Simplex Virus thymidine kinase (HSV tk) promoter, and the 35S promoter from Cauliflower Mosaic Virus. Suitable phage promoters for use with the compositions and methods described herein include, but are not limited to, the E. coli T7 and T3 phage promoters, the S. typhimurium phage SP6 promoter, B. subtilis SP01 phage and B. subtilis phage phi 29 promoters, and N4 phage and K11 phage promoters as described in U.S. Pat. No. 5,547,892, the disclosure of which is incorporated herein by reference.

Upon incorporation of a polynucleotide encoding a target gene has been incorporated into the genome of a cell (e.g., the nuclear genome of a hematopoietic stem cell), the transcription of this polynucleotide can be induced by methods known in the art. For example expression can be induced by exposing the mammalian cell to an external chemical reagent, such as an agent that modulates the binding of a transcription factor and/or RNA polymerase to the mammalian promoter and thus regulate gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use with the compositions and methods described herein include enhancer sequences. Enhancers represent another class of regulatory elements that induce a conformational change in the polynucleotide comprising the gene of interest such that the DNA adopts a three-dimensional orientation that is favorable for binding of transcription factors and RNA polymerase at the transcription initiation site. Thus, polynucleotides for use with the compositions and methods described herein include those that encode a target gene and additionally include a mammalian enhancer sequence. Many enhancer sequences are now known from mammalian genes, and examples include enhancers from the genes that encode mammalian globin, elastase, albumin, α-fetoprotein, and insulin. Enhancers for use with the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al. Nature 297:17 (1982), the disclosure of which is incorporated herein by reference. An enhancer may be spliced into a vector containing a polynucleotide encoding a target gene, for example, at a position 5′ or 3′ to this gene. In a preferred orientation, the enhancer is positioned at the 5′ side of the promoter, which in turn is located 5′ relative to the polynucleotide encoding the target gene.

In addition to promoting high rates of transcription and translation, stable expression of an exogenous gene in a hematopoietic stem cell can be achieved by integration of the polynucleotide comprising the gene into the nuclear DNA of the cell. A variety of vectors for the delivery and integration of polynucleotides encoding exogenous proteins into the nuclear DNA of a mammalian cell have been developed. Examples of expression vectors are disclosed in, e.g., WO94/11026, the disclosure of which is incorporated herein by reference. Expression vectors for use with the compositions and methods described herein contain a polynucleotide sequence that encodes a target gene, as well as, e.g., additional sequence elements used for the expression of these enzymes and/or the integration of these polynucleotide sequences into the genome of a mammalian cell. Certain vectors that can be used for the expression of target genes include plasmids that contain regulatory sequences, such as promoter and enhancer regions, which direct gene transcription. Other useful vectors for expression of target genes contain polynucleotide sequences that enhance the rate of translation of these genes or improve the stability or nuclear export of the mRNA that results from gene transcription. These sequence elements often encode features within RNA transcripts that enhance the nuclear export, cytosolic half-life, and ribosomal affinity of these molecules, e.g., 5′ and 3′ untranslated regions, an internal ribosomal entry site (IRES), and polyadenylation signal site in order to direct efficient transcription of the gene carried on the expression vector. Exemplary expression vectors may also contain a polynucleotide encoding a marker for selection of cells that contain such a vector. Non-limiting examples of a suitable marker include genes that encode resistance to antibiotics, such as ampicillin, chloramphenicol, kanamycin, or nourseothricin.

### Vectors for the Expression of Target Genes

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and often do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picomavirus and alphavirus, and double stranded DNA viruses including herpes virus (e.g., Herpes Simplex virus types 1 and 2. Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses. D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996, the disclosure of which is incorporated herein by reference). Other examples of viral vectors include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described in, e.g., U.S. Pat. No. 5,801,030, the disclosure of which is incorporated herein by reference.

### Additional Transfection Methods

Other techniques that can be used to introduce a polynucleotide, such as DNA or RNA (e.g., mRNA, tRNA, siRNA, miRNA, shRNA, chemically modified RNA) into a mammalian cell are well known in the art. For instance, electroporation can be used to permeabilize mammalian cells by the application of an electrostatic potential. Mammalian cells, such as hematopoietic stem cells, subjected to an external electric field in this manner are subsequently predisposed to the uptake of exogenous nucleic acids. Electroporation of mammalian cells is described in detail, e.g., in Chu et al. Nucleic Acids Research 15:1311 (1987), the disclosure of which is incorporated herein by reference. A similar technique, Nucleofection™, utilizes an applied electric field in order to stimulate the update of exogenous polynucleotides into the nucleus of a eukaryotic cell. Nucleofection™ and protocols useful for performing this technique are described in detail, e.g., in Distler et al. Experimental Dermatology 14:315 (2005), as well as in US 2010/0317114, the disclosures of each of which are incorporated herein by reference.

Additional techniques useful for the transfection of hematopoietic stem cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a hematopoietic stem cell. Squeeze-poration is described in detail, e.g., in Sharei et al. Journal of Visualized Experiments 81:e50980 (2013), the disclosure of which is incorporated herein by reference.

Lipofection represents another technique useful for transfection of hematopoietic stem cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids. e.g., by direct fusion of the liposome with the cell membrane or by endocytosis of the complex. Lipofection is described in detail, e.g., in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Cationic molecules that associate with polynucleotides so as to impart a positive charge favorable for interaction with the cell membrane include activated dendrimers (described, e.g., in Dennig. Topics in Current Chemistry 228:227 (2003), the disclosure of which is incorporated herein by reference) and diethylaminoethyl (DEAE)-dextran, the use of which as a transfection agent is described in detail, e.g., in Gulick et al. Current Protocols in Molecular Biology 40:1:9.2:9.2.1 (1997), the disclosure of which is incorporated herein by reference. Magnetic beads are another tool that can be used to transfect hematopoietic stem cells in a mild and efficient manner, as this methodology utilizes an applied magnetic field in order to direct the uptake of nucleic acids. This technology is described in detail, e.g., in US 2010/0227406, the disclosure of which is incorporated herein by reference.

Another useful tool for inducing the uptake of exogenous nucleic acids by hematopoietic stem cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al. Methods in Cell Biology 82:309 (2007), the disclosure of which is incorporated herein by reference.

Microvesicles represent another potential vehicle that can be used to modify the genome of a hematopoietic stem cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al. Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

In addition to viral vectors, a variety of additional tools have been developed that can be used for the incorporation of exogenous genes into hematopoietic stem cells. One such method that can be used for incorporating polynucleotides encoding target genes into hematopoietic stem cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US2005/0112764), the disclosures of each of which are incorporated herein by reference.

Another useful tool for the disruption and integration of target genes into the genome of a hematopoietic stem cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing hematopoietic stem cell genomes in order to cleave DNA prior to the incorporation of a gene encoding a target gene. The use of CRISPR/Cas to modulate gene expression has been described in. e.g., U.S. Pat. No. 8,697,359, the disclosure of which is incorporated herein by reference.

The CRISPR/Cas system can be used to create one or more double stranded breaks in a target DNA sequence, which can then be repaired by either the homologous recombination (HR) or non-homologous end joining (NHEJ) DNA repair pathways. The Cas9 enzyme, together with a guide RNA specific to the target DNA (gRNA), can be supplied to a cell to induce one or more double strand breask. The Cas9 enzyme can be supplied as a protein, as a ribonucleoprotein complexed with the guide RNA, or as an RNA or DNA encoding the Cas9 protein that is then used by the cell to synthesize the Cas9 protein. The gRNA may comprise both tracrRNA and crRNA sequences in a chimeric RNA. Alternatively, or in addition, the gRNA may comprise a scaffold region that binds to the Cas9 protein, and a complementary base pairing region, also sometimes called a spacer, that targets the gRNA Cas9 protein complex to a particular DNA sequence. In some cases, the complementary base pairing region can be about 20 nucleotides in length, and is complementary to target DNA sequence immediately adjacent to a protospacer adjacent motif (e.g., a PAM motif). In some cases, the PAM comprises a sequence of NGG, NGA or NAG. The complementary base pairing region of the gRNA hybridizes to the target DNA sequence, and guides the gRNA Cas9 protein complex to the target sequence where the Cas9 endonuclease domains then cut within the target sequence, generating a double strand break that may be 3-4 nucleotides upstream of the PAM. Thus, by altering the complementary base pairing region, almost any DNA sequence can be targeted for the generation of a double stranded break. Methods for selecting an appropriate complementary base pairing region will be known to those skilled in the art. For example, gRNAs can be selected to minimize the number of off-target binding sites of the gRNA in the target DNA sequence. In some cases, modified Cas9 genome editing systems may be used to, for example, increase DNA targeting specificity. An example of a modified Cas9 genome editing system comprises split Cas9 systems such as the Dimeric Cas9-Fok1 genome editing system.

The double strand break or breaks generated by CRISPR/Cas9 genome editing system may be repaired by the non homologous end joining pathway (NHEJ), which ligates the ends of the double strand break together. NHEJ may result in deletions in the DNA around or near the site of the double strand break. Alternatively, the double strand break generated by CRISPR/Cas9 genome editing system may be repaired through a homology directed repair, also called homologous recombination (HR) repair pathway. In the HR pathway, the double strand break is repaired by exchanging sequences between two similar or identical DNA molecules. The HR repair pathway can therefore be used to introduce exogenous DNA sequences into the genome. In using the HR pathway for genome editing, a DNA template is supplied to the cell along with the Cas9 and gRNA. In some cases, the template may contain exogenous sequences to be introduced into the genome via genome editing flanked by homology arms that comprise DNA sequences on either side of the site of the Cas9 induced double strand break. These homology arms may be, for example, between about 50 or 1000 nucleotides, or in other cases up to several kilobases in length or longer. The template may be a linear DNA, or a circular DNA such as a plasmid, or may be supplied using a viral vector or other means of delivery. The template DNA may comprise double stranded or single stranded DNA. All manner of delivering the template DNA, the gRNA and the Cas9 protein to the cell to achieve the desired genome editing are envisaged as being within the scope of the invention.

The CRISPR/Cas9 and HR based genome editing systems of the disclosure provide not only methods of introducing exogenous DNA sequences into a genome or DNA sequence of interest, but also a platform for correcting mutations in genes. An altered or corrected version of a mutated sequence, for example a sequence changing one or more point mutations back to the wild type consensus sequence, inserting a deleted sequence, or deleting an inserted sequence, could be supplied to the cell as a template sequence, and that template sequence used by the cell to fix a CRISPR/Cas9 induced double strand break via the HR pathway. For example, in a patient with one or more disease causing mutations, hematopoietic stem and/or progenitor cells such as the hematopoietic stem and/or progenitor cells of the patient, can be removed from the body. The mutation can then corrected by CRISPR/Cas9 and HR mediated genome editing in the genome of one or more of these hematopoietic stem and/or progenitor cells, the corrected hematopoietic stem and/or progenitor cell(s) expanded with the methods of the disclosure, and then the edited cell population infused back into the patient, thereby supplying a source of the wild type version of the gene and curing the patient of the disease caused by the mutation or mutations in that gene. Mutations that can cause genetic diseases include not only point mutations, but also insertions, deletions and inversions. These mutations can be in protein coding sequence and affect the amino acid sequence of the protein, or they may be in non-coding sequences such as untranslated regions, promoters, cis regulatory elements required for gene expression, sequences required for splicing, or sequences required for DNA structure. All mutations are potentially editable by CRISPR/Cas9 mediated genome editing methods of the disclosure. In some cases, the patient may be conditioned to eliminate or reduce the native hematopoietic stem and/or progenitor cells that carry the mutant version of the gene, thus enriching for the exogenously supplied genome edited hematopoietic stem and/or progenitor cells. Both autologous and allogeneic genome edited hematopoietic stem and/or progenitor cells can be used to treat a genetic disease of a patient of the disclosure.

In addition to the CRISPR/Cas9 system, alternative methods for disruption of a target DNA by site-specifically cleaving genomic DNA prior to the incorporation of a gene of interest in a hematopoietic stem and/or progenitor cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Umov et al. Nature Reviews Genetics 11:636 (2010); and in Joung et al. Nature Reviews Molecular Cell Biology 14:49 (2013), the disclosure of both of which are incorporated herein by reference. As with the CRISPR/Cas9 genome editing systems, double strand breaks introduced by TALENS or ZFNs can also repaired via the HR pathway, and this pathway can be used to introduce exogenous DNA sequences or repair mutations in the DNA.

Additional genome editing techniques that can be used to disrupt or incorporate polynucleotides encoding target genes into the genome of a hematopoietic stem cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a hematopoietic stem cell. These single-chain nucleases have been described extensively in, e.g., U.S. Pat. Nos. 8,021,867 and 8,445,251, the disclosures of each of which are incorporated herein by reference.

### Methods for Expanding Hematopoietic Stem Cells

In some aspects, the disclosure features a method of producing an expanded population of hematopoietic stem cells ex vivo, the method including contacting a population of hematopoietic stem cells with the compound of any one of the above aspects or embodiments in an amount sufficient to produce an expanded population of hematopoietic stem cells.

In some aspects, the disclosure features a method of enriching a population of cells with hematopoietic stem cells ex vivo, the method including contacting a population of hematopoietic stem cells with the compound of any one of the above aspects or embodiments in an amount sufficient to produce a population of cells enriched with hematopoietic stem cells.

In some aspects, the disclosure features a method of maintaining the hematopoietic stem cell functional potential of a population of hematopoietic stem cells ex vivo for two or more days, the method including contacting a first population of hematopoietic stem cells with the compound of any one of the above aspects or embodiments, wherein the first population of hematopoietic stem cells exhibits a hematopoietic stem cell functional potential after two or more days that is greater than that of a control population of hematopoietic stem cells cultured under the same conditions and for the same time as the first population of hematopoietic stem cells but not contacted with the compound.

In some embodiments, said method for expanding hematopoietic stem cells, comprises (a) providing a starting cell population comprising hematopoetic stem cells and (b) culturing said starting cell population ex vivo in the presence of an AHR antagonist agent compound of any one of the above aspects or embodiments.

The starting cell population comprising hematopoietic stem cells will be selected by the person skilled in the art depending on the envisaged use. Various sources of cells comprising hematopoietic stem cells have been described in the art, including bone marrow, peripheral blood, neonatal umbilical cord blood, placenta or other sources such as liver, particularly fetal liver.

The cell population may first be subjected to enrichment or purification steps, including negative and/or positive selection of cells based on specific cellular markers in order to provide the starting cell population. Methods for isolating said starting cell population based on specific cellular markers may use fluorescent activated cell sorting (FACS) technology also called flow cytometry or solid or insoluble substrate to which is bound antibodies or ligands that interact with specific cell surface markers. For example, cells may be contacted with a solid substrate (e.g., column of beads, flasks, magnetic particles) containing the antibodies and any unbound cells are removed. When a solid substrate comprising magnetic or paramagnetic beads is used, cells bound to the beads can be readily isolated by a magnetic separator.

In some embodiments, said starting cell population is enriched in a desirable cell marker phenotype (e.g., CD34+, CD133+, CD90+) or based on efflux of dyes such as rhodamine, Hoechst or aldehyde dehydrogenase activity. In some embodiments, said starting cell population is enriched in CD34+ cells. Methods for enriching blood cell population in CD34+ cells include kits commercialized by Miltenyi Biotec (CD34+ direct isolation kit, Miltenyi Biotec, Bergisch, Gladbach, Germany) or by Baxter (Isolex 3000).

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoietic stem cells. In some embodiments, the hematopoietic stem cells are CD34+CD90+CD45RA− hematopoietic stem cells.

In some embodiments, the hematopoietic stem cells are mammalian cells, such as human cells. In some embodiments, the human cells are CD34+ cells, such as CD34+ cells are CD34+, CD34+CD38−, CD34+CD38−CD90+, CD34+CD38−CD90+CD45RA−, CD34+CD38−CD90+CD45RA−CD49F+, or CD34+CD90+CD45RA− cells.

In some embodiments, the hematopoietic stem cells are obtained from human cord blood, mobilized human peripheral blood, or human bone marrow. The hematopoietic stem cells may, for example, be freshly isolated from the human or may have been previously cryopreserved.

The amount of cord blood from a single birth is often inadequate to treat an adult or an older child. One advantage of the expansion methods using the compounds of the disclosure, or an agent capable of down-regulating the activity and/or expression of aryl hydrocarbon receptor and/or a downstream effector of aryl hydrocarbon receptor pathway, is that it enables the production of a sufficient amount of hematopoietic stem cells from only one cord blood unit.

Accordingly, in some embodiments, the starting cell population is derived from neonatal umbilical cord blood cells which have been enriched in CD34+ cells. In some embodiments, said starting cell population is derived from one or two umbilical corm blood units.

In some embodiments, the starting cell population is derived from human mobilized peripheral blood cells which nave been enriched in CD34+ cells. In some embodiments, said starting cell population is derived from human mobilized peripheral blood cells isolated from only one patient.

Said starting cell population enriched in CD34+ cells may preferably contain at least about 50% CD34+ cells, in some embodiments, more than about 90% CD34+ cells, and may comprise between 10⁵ and 10⁹ nucleated cells.

The starting cell population may be used directly for expansion or frozen and stored for use at a later date.

Conditions for culturing the starting cell population for hematopoietic stem cell expansion will vary depending, inter alia, on the starting cell population, the desired final number of cells, and desired final proportion of HSCs.

In some embodiments, the culturing conditions comprises the use of other cytokines and growth factors, generally known in the art for hematopoietic stem cell expansion. Such cytokines and growth factors include without limitation IL-1, IL-3, IL-6, IL-11, G-CSF, GM-CSF, SCF, FIT3-L, thrombopoietin (TPO), erythropoeitin, and analogs thereof. As used herein, “analogs” include any structural variants of the cytokines and growth factors having the biological activity as the naturally occurring forms, including without limitation, variants with enhanced or decreased biological activity when compared to the naturally occurring forms or cytokine receptor agonists such as an agonist antibody against the TPO receptor (for example, VB22B sc(Fv)2 as detailed in patent publication WO 2007/145227, and the like). Cytokine and growth factor combinations are chosen to expand HSC and progenitor cells while limiting the production of terminally differentiated cells. In some embodiments, one or more cytokines and growth factors are selected from the group consisting of SCF, Flt3-L and TPO. In some embodiments, at least TPO is used in a serum-free medium under suitable conditions for HSC expansion. In some embodiments, a mixture of IL6, SCF, Flt3-L and TPO is used in the method for expanding HSCs in combination with the compound of the present disclosure.

The expansion of HSC may be carried out in a basal medium, which may be supplemented with mixtures of cytokines and growth factors. A basal medium typically comprises amino acids, carbon sources, vitamins, serum proteins (e.g. albumin), inorganic salts, divalent cations, buffers and any other element suitable for use in expansion of HSC. Examples of such basal medium appropriate for a method of expanding HSC include, without limitation, StemSpan® SFEM-Serum-Free Expansion Medium (StemCell Technologies, Vancouver, Canada), StemSpan® H3000-Defined Medium (StemCell Technologies, Vancouver, Canada), CellGro® SCGM (CellGenix, Freiburg Germany), StemPro®-34 SFM (Invitrogen).

In some embodiments, the compound of the present disclosure is administered during the expansion method of said starting cell population under a concentration appropriate for HSC expansion. In some embodiments, said compound or AHR modulating agent is administered at a concentration comprised between 1 pM and 100 μM, for example between 10 pM and 10 μM, or between 100 pM and 1 μM.

In some embodiments where starting cell population essentially consists of CD34+ enriched cells from one or two cord blood units, the cells are grown under conditions for HSC expansion from about 3 days to about 90 days, for example between 7 and 2 days and/or until the indicated fold expansion and the characteristic cell populations are obtained. In some embodiments, the cells are grown under conditions for HSC expansion not more than 21 days, 14 days or 7 days.

In some embodiments, the starting cell population is cultured during a time sufficient to reach an absolute number of CD34+ cells of at least 10⁵, 10⁶, 10⁷, 10⁸ or 10⁹ cells. In some embodiments, said starting cell population is cultured during a time sufficient for a 10 to 50000 fold expansion of CD34+ cells, for example between 100 and 10000 fold expansion, for examples between 50 and 1000 fold expansion.

The cell population obtained after the expansion method may be used without further purification or may be subject to further purification or selection steps.

The cell population may then be washed to remove the compound of the present disclosure and/or any other components of the cell culture and resuspended in an appropriate cell suspension medium for short term use or in a long-term storage medium, for example a medium suitable for cryopreservation.

In some embodiments, the hematopoietic stem or progenitor cells are expanded ex vivo prior to infusion into the patient.

In some embodiments, the hematopoietic stem or progenitor cells are expanded ex vivo prior to infusion into the patient by contacting the hematopoietic stem or progenitor cells with at least one agent selected from the group consisting of an aryl hydrocarbon receptor antagonist, nicotinamide, UM729, and UM171.

In some embodiments, the expanded cord blood or population of hematopoietic stem or progenitor cells is selected from the group consisting of MGTA-456, omidubicel (NiCord), and ECT-001.

In some embodiments, the expanded cord blood or population of hematopoietic stem or progenitor cells is MGTA-456.

In some embodiments, the hematopoietic stem or progenitor cells are expanded ex vivo by contacting the hematopoietic stem or progenitor cells with an aryl hydrocarbon receptor antagonist.

In some embodiments, the aryl hydrocarbon receptor antagonist is SR-1.

In some embodiments, the aryl hydrocarbon receptor antagonist is compound 2.

### Aryl Hydrocarbon Receptor Antagonists

Prior to infusion into a patient, hematopoietic and progenitor cells may be expanded ex vivo, for instance, by treatment with an aryl hydrocarbon receptor antagonist. Aryl hydrocarbon receptor antagonists useful in conjunction with the compositions and methods described herein include those described in U.S. Pat. No. 9,580,426, the disclosure of which is incorporated herein by reference in its entirety.

In some embodiments, the aryl hydrocarbon receptor antagonist is a compound represented by formula (I)

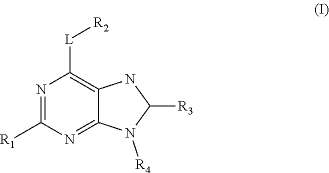

or a pharmaceutically acceptable salt, hydrate, or solvate thereof, wherein:

L is selected from —NR₅ₐ(CH₂)₂₋₃, —NR₅ₐ(CH₂)₂NR_{5b}—, —NR₅ₐ(CH₂)₂S—, —NR₅ₐCH₂CH(OH)— and —NR₅ₐCH(CH₃)CH₂—; wherein R₅ₐ and R_{5b} are independently selected from hydrogen and C₁₋₄ alkyl;

R₁ is selected from thiophenyl, 1H-benzoimidazolyl, isoquinolinyl, 1H-imidazopyridinyl, benzothiophenyl, pyrimidinyl, pyridinyl, pyrazinyl, pyridazinyl, and thiazolyl; for instance, wherein the thiophenyl, 1H-benzoimidazolyl, isoquinolinyl, 1H-imidazopyridinyl, benzothiophenyl, pyrimidinyl, pyridinyl, pyrazinyl, pyridazinyl, or thiazolyl of R₁ can be optionally substituted by 1 to 3 radicals independently selected from cyano, hydroxy, C₁₋₄ alkyl, C₁₋₄ alkoxy, halo, halo-substituted-C₁₋₄ alkyl, halo-substituted-C₁₋₄ alkoxy, amino, —C(O)R₅ₐ, —S(O)₀₋₂R₅ₐ, —C(O)OR₅ₐ and —C(O)NR₅ₐR_{5b}; wherein R₅ₐ and R_{5b} are independently selected from hydrogen and C₁₋₄ alkyl;

R₂ is selected from —S(O)₂NR₈ₐR_{8b}, —NR₈ₐC(O)R_{8b}—, —NR₈ₐC(O)NR_{8b}R_{8c}, phenyl, 1H-pyrrolopyridin-3-yl, 1H-pyrrolopyridin-8-yl, 1H-indolyl thiophenyl, pyridinyl, 1H-1,2,4-triazolyl, 2-oxoimidazolidinyl, 1H-pyrazolyl, 2-oxo-2,3-dihydro-1H-benzoimidazolyl and 1H-indazolyl; wherein R₈ₐ, R_{8b} and R_{8c} are independently selected from hydrogen and C₁₋₄ alkyl; and the phenyl, 1H-pyrrolopyridin-3-yl, 1H-pyrrolo[2,3-b]pyridin-5-yl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl or 1H-indazolyl of R₂ is optionally substituted with 1 to 3 radicals independently selected from hydroxy, halo, methyl, methoxy, amino, —O(CH₂)₂NR₇ₐR_{7b}, —S(O)₂NR₇ₐR_{7b}, —OS(O)₂NR₇ₐR_{7b} and —NR₇ₐS(O)₂R_{7b}; wherein R₇ₐ and R_{7b} are independently selected from hydrogen and C₁₋₄ alkyl;

R₃ is selected from hydrogen, C₁₋₄ alkyl and biphenyl; and

R₄ is selected from C₁₋₁₀ alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, and benzyl, (4-pentylphenyl)(phenyl)methyl and 1-(1-(2-oxo-6,9,12-trioxa-3-azatetradecan-14-yl)-1H-1,2,3-triazol-4-yl)ethyl wherein said alkyl, cyclopropyl, cyclohexyl, 2-(2-oxopyrrolidin-1-yl)ethyl, oxetan-3-yl, oxetan-2-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl or 1-(1-(2-oxo-6,9,12-trioxa-3-azatetradecan-14-yl)-1H-1,2,3-triazol-4-yl)ethyl can be optionally substituted with 1 to 3 radicals independently selected from hydroxy, C₁₋₄ alkyl and halo-substituted-C₁₋₄ alkyl.

In some embodiments, the aryl hydrocarbon receptor antagonist is SR-1, represented by formula (1), below, or a pharmaceutically acceptable salt, hydrate, or solvate thereof.

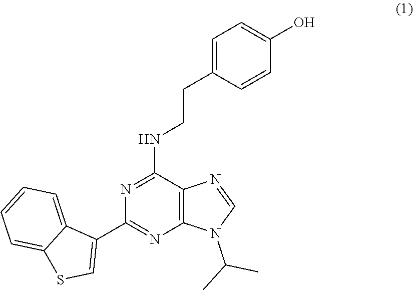 [SMILES: CC(C)n1cnc2c(NCCc3ccc(O)cc3)nc(-c3csc4ccccc34)nc21]

In some embodiments, the aryl hydrocarbon receptor antagonist is Compound 2, represented by formula (2), below, or a pharmaceutically acceptable salt, hydrate, or solvate thereof.

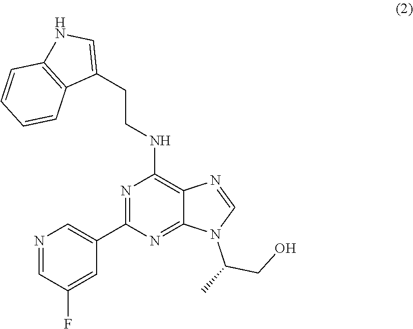 [SMILES: C[C@@H](CO)n1cnc2c(NCCc3c[nH]c4ccccc34)nc(-c3cncc(F)c3)nc21]

In some embodiments, the aryl hydrocarbon receptor antagonist is Compound 2-ent, represented by formula (2-ent), below, or a pharmaceutically acceptable salt, hydrate, or solvate thereof.

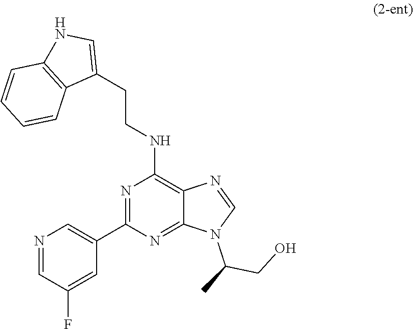 [SMILES: C[C@H](CO)n1cnc2c(NCCc3c[nH]c4ccccc34)nc(-c3cncc(F)c3)nc21]

In some embodiments, the aryl hydrocarbon receptor antagonist is Compound 2-rac, represented by formula (2-rac), below, or a pharmaceutically acceptable salt, hydrate, or solvate thereof.

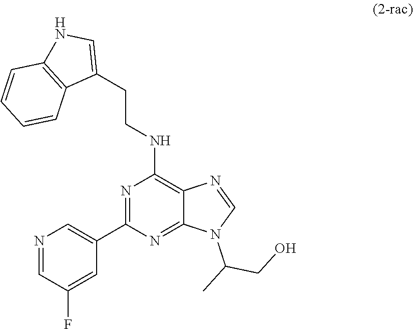 [SMILES: CC(CO)n1cnc2c(NCCc3c[nH]c4ccccc34)nc(-c3cncc(F)c3)nc21]

In some embodiments, the aryl hydrocarbon receptor antagonist is compound represented by formula (IV)

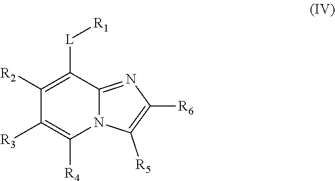

or a pharmaceutically acceptable salt, hydrate, or solvate thereof, wherein:

L is a linker selected from the group consisting of —NR₇ₐ(CR₈ₐR_{8b})ₙ—, —O(CR₈ₐR_{8b})ₙ—, —C(O)(CR₈ₐR_{8b})ₙ, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, —NR₇ₐ(CR₈ₐR_{8b})ₙNR₇ₐ—, —NR₇ₐ(CR₈ₐR_{8b})ₙO—, —NR₇ₐ(CR₈ₐR_{8b})ₙS—, —O(CR₈ₐR_{8b})ₙNR₇ₐ—, —O(CR₈ₐR_{8b})ₙO—, —O(CR₈ₐR_{8b})ₙS—, —S(CR₈ₐR_{8b})ₙNR₇ₐ—, —S(CR₈ₐR_{8b})ₙO—, —S(CR₈ₐR_{8b})ₙS—, and —NR₇ₐC(O)O(CR₈ₐR_{8b})—, wherein R₇ₐ, R_{7b}, R₈ₐ and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl:

R₂ is selected from the group consisting of hydrogen and optionally substituted C1-4 alkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

As used herein to describe linkers (represented by “L” in formulas (IV), (V), and the like), the notation “-(Linker)-” (wherein “linker” is represented using chemical symbols such as NR₇ₐ(CR₈ₐR_{8b})ₙ, O(CR₈ₐR_{8b})ₙ, C(O)(CR₈ₐR_{8b})ₙ, C(S)(CR₈ₐR_{8b})ₙ, S(O)₀₋₂(CR₈ₐR_{8b})ₙ, (CR₈ₐR_{8b})ₙ, —NR₇ₐC(O)(CR₈ₐR_{8b})ₙ, NR₇ₐC(S)(CR₈ₐR_{8b})ₙ, OC(O)(CR₈ₐR_{8b})ₙ, OC(S)(CR₈ₐR_{8b})ₙ, C(O)NR₇ₐ(CR₈ₐR_{8b})ₙ—, C(S)NR₇ₐ(CR₈ₐR_{8b})ₙ, C(O)O(CR₈ₐR_{8b})ₙ, C(S)O(CR₈ₐR_{8b})ₙ, S(O)₂NR₇ₐ(CR₈ₐR_{8b})ₙ. NR₇ₐS(O)₂(CR₈ₐR_{8b})ₙ, and NR₇ₐC(O)NR_{7b}(CR₈ₐR_{8b})ₙ) designates that the left hyphen represents a covalent bond to the indicated position on the imidazopyridine or imidazopyrazine ring system, while the right hyphen represents a covalent bond to R₁.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, —OC(S)NR₉ₐR_{9b}R_{9c}, phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, and —OC(S)CR₉ₐR_{9b}R_{9c}.

In some embodiments, R₁ is selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, R₁ is selected from the group consisting of:

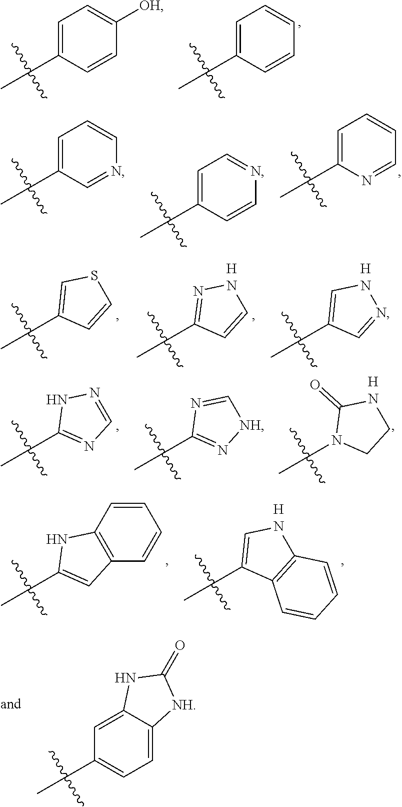

In some embodiments, R₁ is selected from the group consisting of:

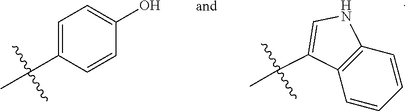 [SMILES: *c1ccc(O)cc1 ; "and" ; *c1c[nH]c2ccccc12 ; "."]

In some embodiments, R₁ is selected from the group consisting of phenol-4-yl and 1H-indol-3-yl.

In some embodiments, L is selected from the group consisting of —NR₇ₐ(CR₈ₐR_{8b})ₙ— and —O(CR₈ₐR_{8b})ₙ—.

In some embodiments, L is selected from the group consisting of —NH(CH₂)₂— and —O(CH₂)₂—.

In some embodiments, R₂ is hydrogen.

In some embodiments, R₃ is selected from the group consisting of optionally substituted aryl and optionally substituted heteroaryl.

In some embodiments, R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted, for example, with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄ alkyl.

In some embodiments, R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted, for example, with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein the thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted, for example, with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of optionally substituted:

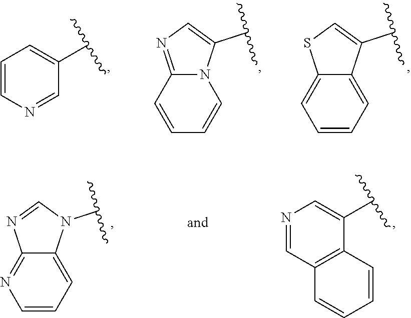 [SMILES: *c1cccnc1 ; *c1cnc2ccccn12 ; *c1csc2ccccc12 ; *n1cnc2ncccc21 ; *c1cncc2ccccc12]

In some embodiments, R₃ is pyridin-3-yl, wherein the pyridin-3-yl is optionally substituted at C5, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, the pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

In some embodiments, R₃ is selected from the group consisting of:

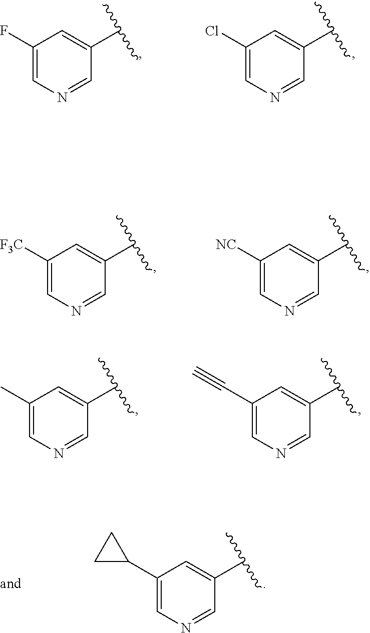

In some embodiments, R₃ is imidazo[1,2-a]pyridin-3-yl, wherein the imidazo[1,2-a]pyridin-3-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is benzo[b]thiophen-3-yl, wherein the benzo[b]thiophen-3-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is 1H-imidazo[4,5-b]pyridin-1-yl, wherein the 1H-imidazo[4,5-b]pyridin-1-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is isoquinolin-4-yl, wherein the isoquinolin-4-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₄ is hydrogen.

In some embodiments, R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted, for example, with from 1 to 3 substituents independently selected from the group consisting of hydroxy, C1-4 alkyl, and halo-substituted-C1-4 alkyl.

In some embodiments, R₃ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

In some embodiments, R₅ is (S)-1-hydroxypropan-2-yl.

In some embodiments, R₅ is (R)-1-hydroxypropan-2-yl

In some embodiments, R₅ is (S)-sec-butyl.

In some embodiments, R₅ is (R)-sec-butyl.

In some embodiments, R₅ is selected from the group consisting of (i), (ii), (iii), (iv), and (v)

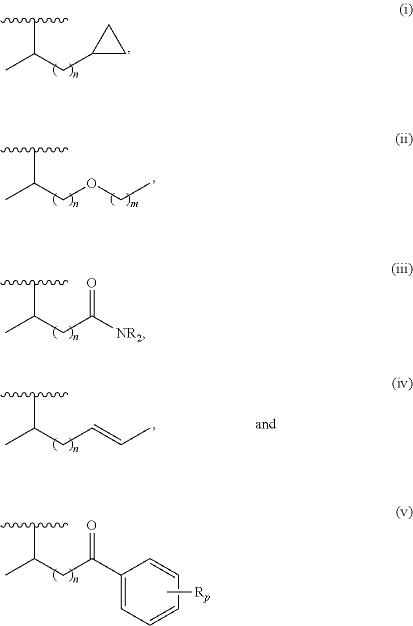

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

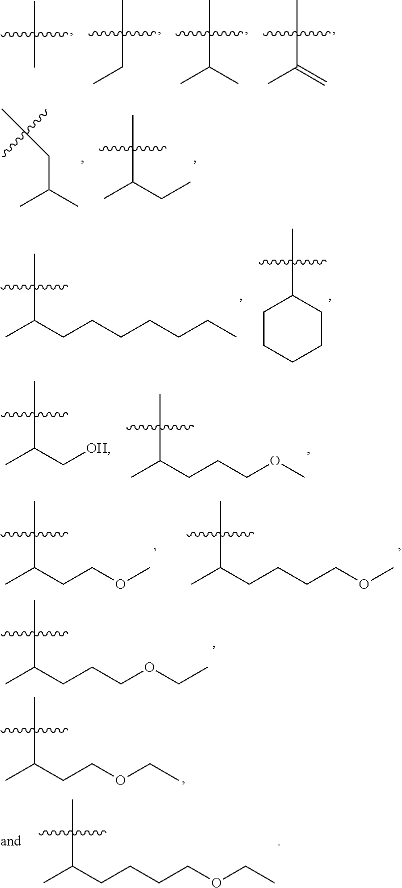

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, R₅ is (S)-4-methoxybutan-2-yl.

In some embodiments, R₅ is (R)-4-methoxybutan-2-yl.

In some embodiments, R₅ is (S)-5-methoxypentan-2-yl.

In some embodiments, R₅ is (R)-5-methoxypentan-2-yl.

In some embodiments, R₅ is (S)-4-ethoxybutan-2-yl.

In some embodiments, R₅ is (R)-4-ethoxybutan-2-yl.

In some embodiments, R₈ is hydrogen.

In some embodiments, the aryl hydrocarbon receptor antagonist is a compound represented by formula (IV-a)

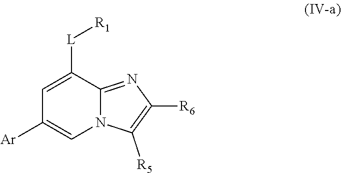

or a pharmaceutically acceptable salt, hydrate, or solvate thereof, wherein:

L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl (for example, R₁ may be selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl);

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, Ar is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound by formula (IV-b)

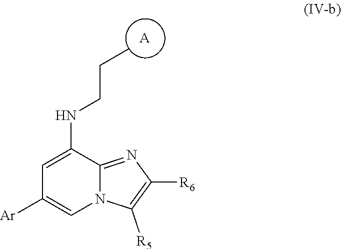

or a pharmaceutically acceptable salt, hydrate, or solvate thereof, wherein:

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, A is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, A is selected from the group consisting of phenol-4-yl and 1H-indol-3-yl.

In some embodiments, the aryl hydrocarbon receptor antagonist is a compound represented by formula (IV-c)

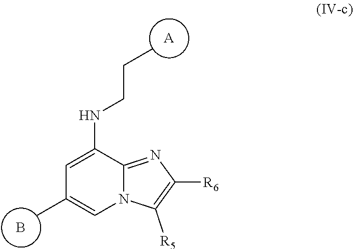

or a pharmaceutically acceptable salt, hydrate, or solvate thereof, wherein:

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, B is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (IV-d)

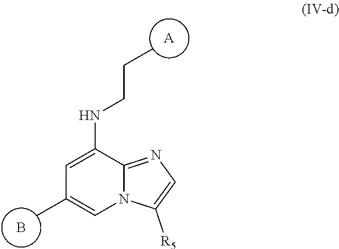

or a pharmaceutically acceptable salt, hydrate, or solvate thereof, wherein:

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl.

In some embodiments, the aryl hydrocarbon receptor antagonist is a compound represented by formula (IV-e)

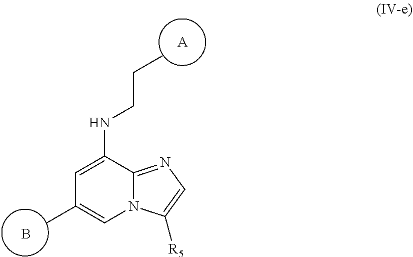

or a pharmaceutically acceptable salt, hydrate, or solvate thereof, wherein:

A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy. C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₃ is selected from the group consisting of (i), (ii), (iii), (iv), and (v)

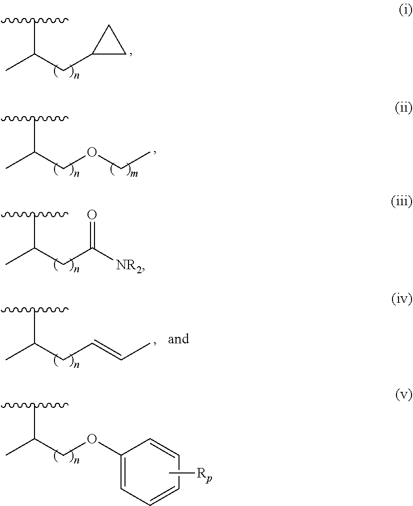

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₃ is selected from the group consisting of:

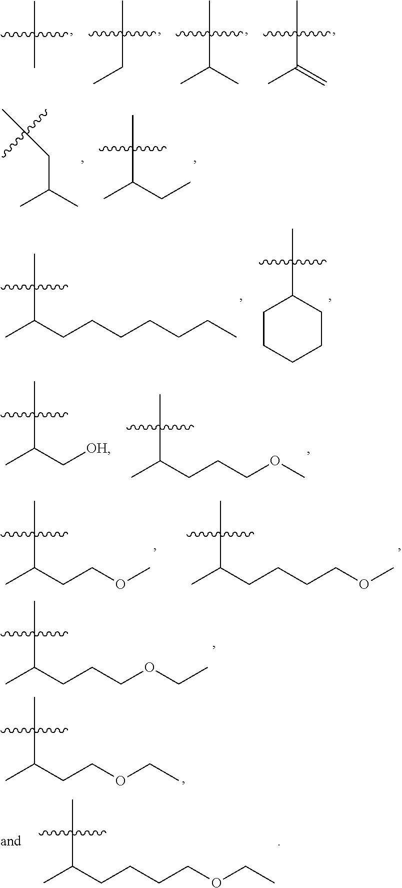

In some embodiments, R₅ is (ii).

In some embodiments, R₃ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-8-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (IV-f)

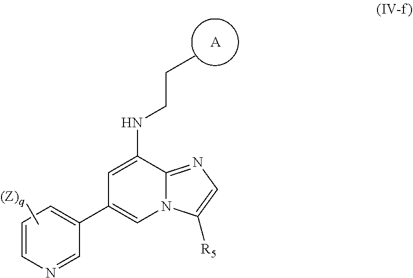

or a pharmaceutically acceptable salt, hydrate, or solvate thereof, wherein

A is an optionally substituted ring system selected from the group consisting of phenol-4-yl and 1H-indol-3-yl;

q is an integer from 0 to 4;

each Z is independently a substituent selected from the group consisting of C1-4 alkyl, halo, halo-substituted-C1-4 alkyl, C2-4 alkenyl, C2-4 alkynyl, C3-8 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₃ is selected from the group consisting of (i), (ii), (iii), (iv), and (v)

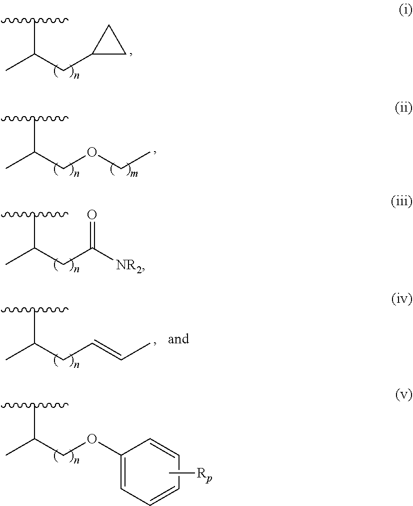

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

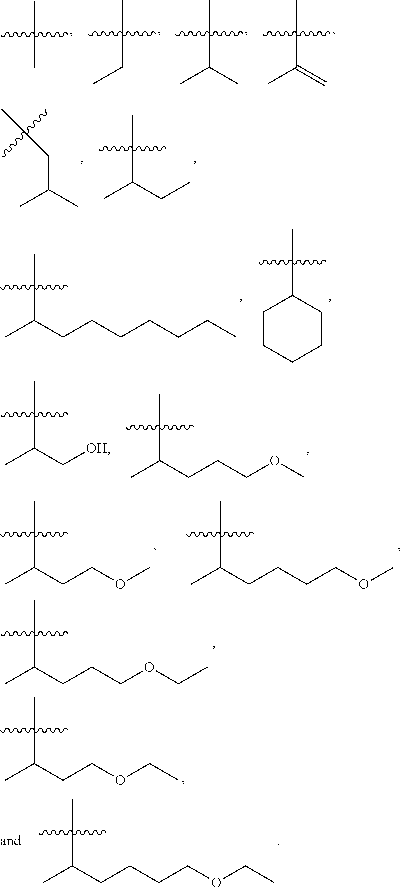

In some embodiments, R₅ is (ii);

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, each Z is independently a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (IV-g)

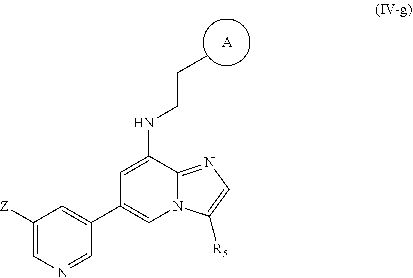

or a pharmaceutically acceptable salt, hydrate, or solvate thereof, wherein:

A is an optionally substituted ring system selected from the group consisting of phenol-4-yl and 1H-indol-3-yl;

Z is a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₃ is selected from the group consisting of (i), (ii), (iii), (iv), and (v)

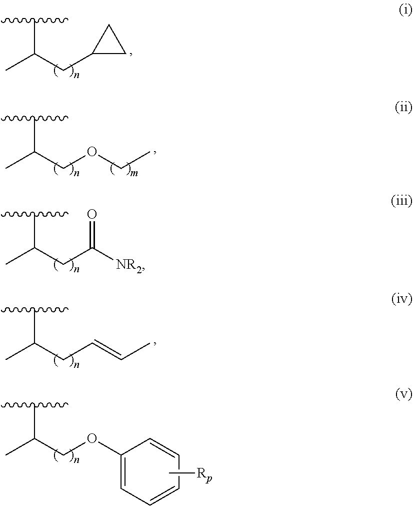

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

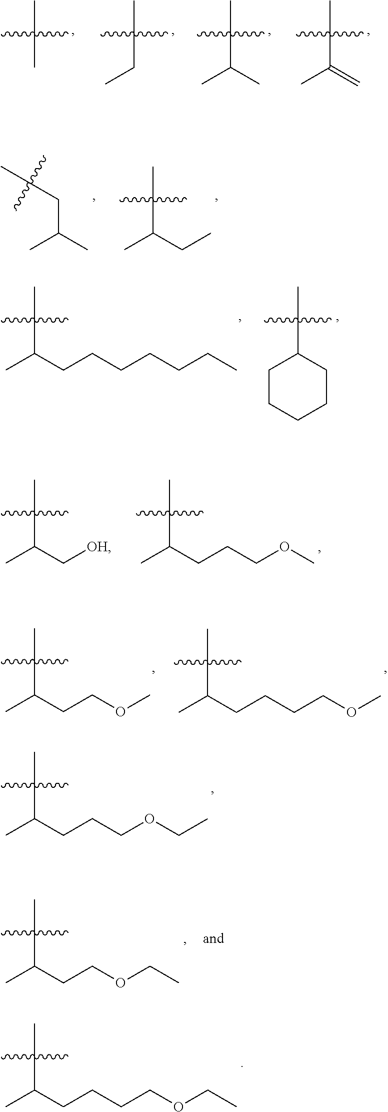

In some embodiments, R₅ is (ii).

In some embodiments, R₃ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-8-methoxyhexan-2-yl, 6-ethoxyhexan-2-yl, (S)-8-ethoxyhexan-2-yl, and (R)-6-ethoxyhexan-2-yl.

In some embodiments, the aryl hydrocarbon receptor antagonist is a compound represented by formula (IV-h)

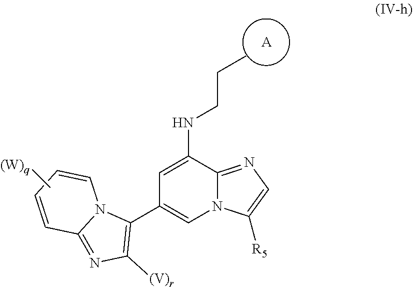

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₅ is selected from the group consisting of (i), (ii), (iii), (iv), and (v)

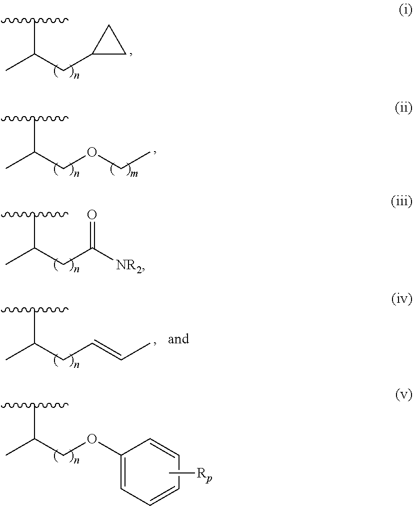

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

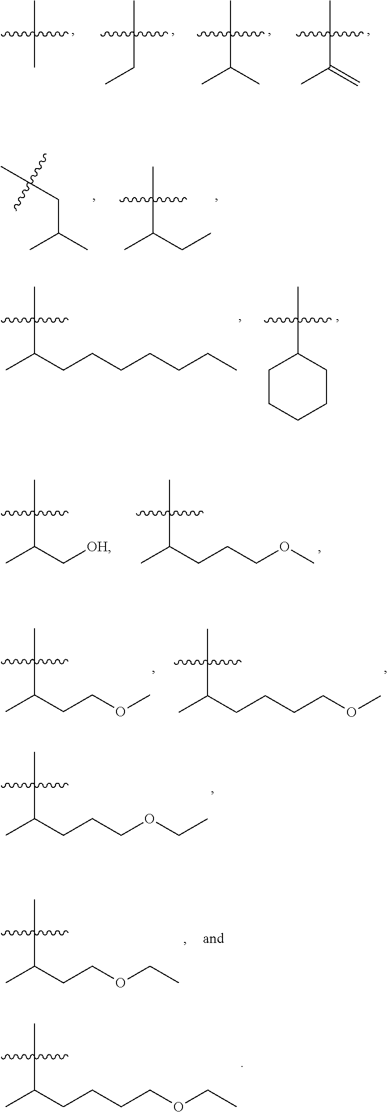

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (IV-i)

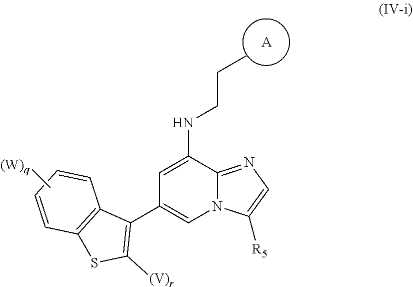

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₃ is selected from the group consisting of (i), (ii), (iii), (iv), and (v)

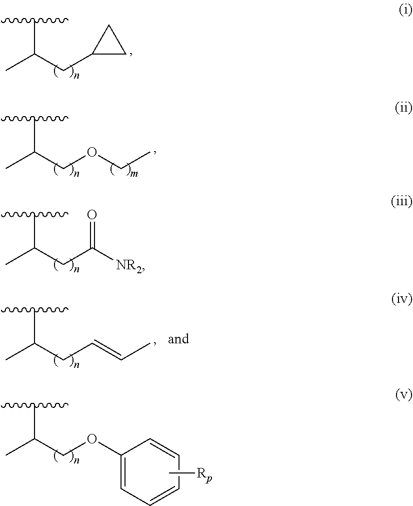

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

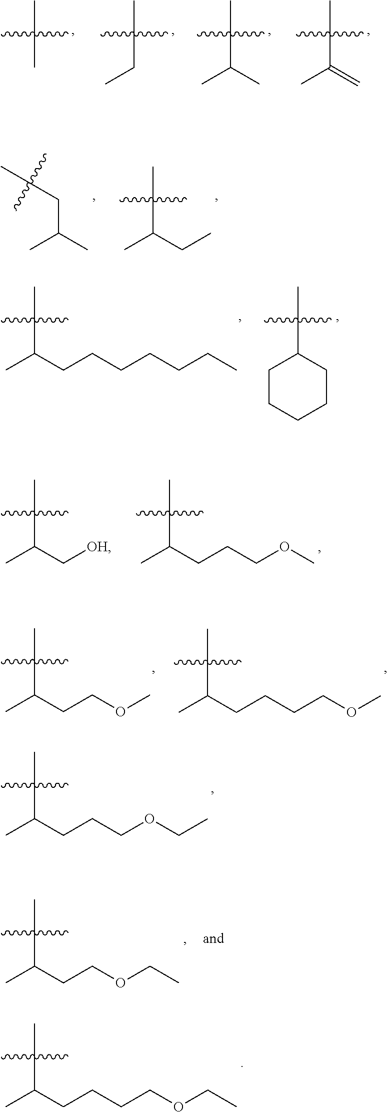

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (IV-j)

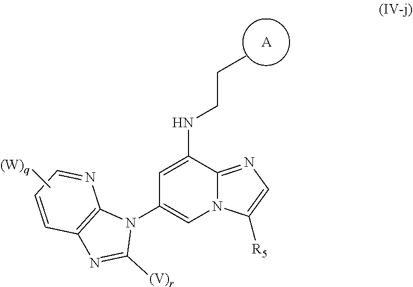

or a pharmaceutically acceptable salt, hydrate, or solvate thereof, wherein

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₉ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₅ is selected from the group consisting of (i), (ii), (iii), (iv), and (v)

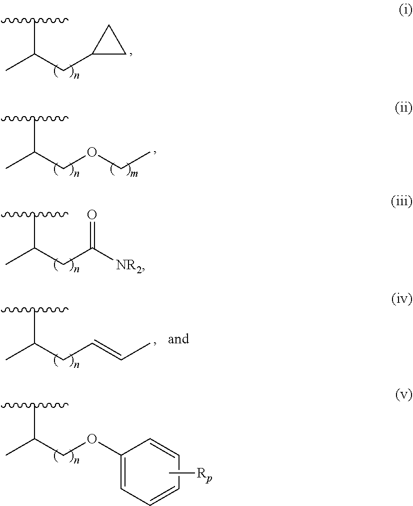

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

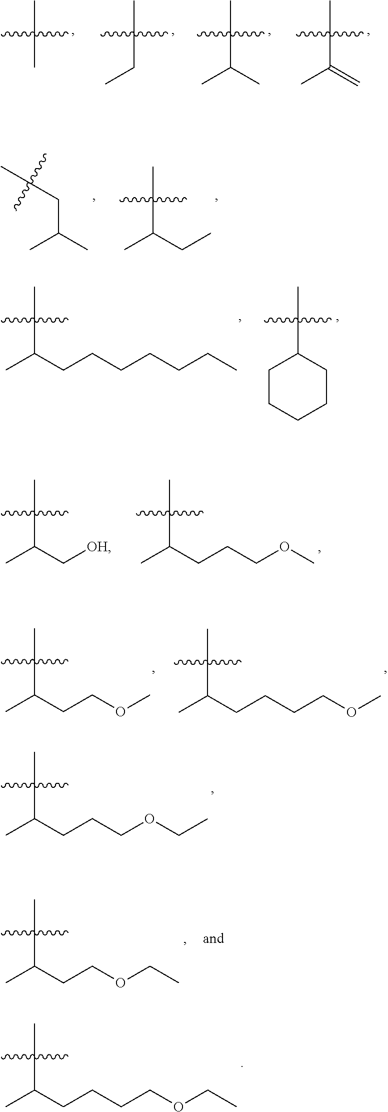

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (IV-k)

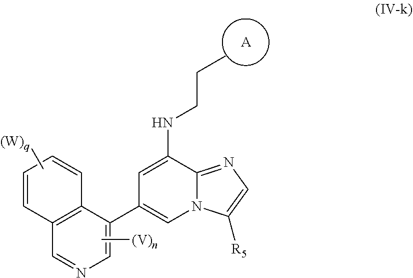

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl. C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₅ is selected from the group consisting of (i), (ii), (iii), (iv), and (v)

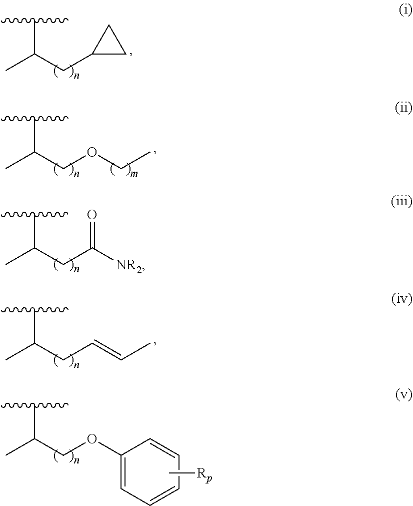

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

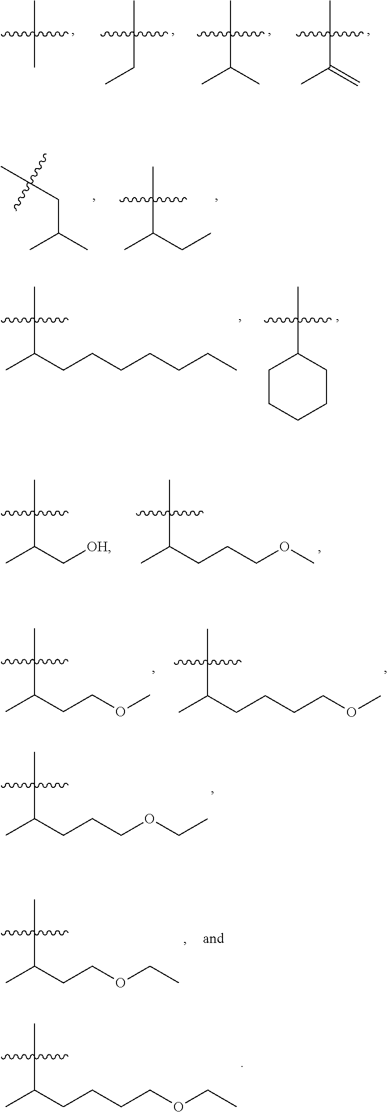

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is compound (3), compound (4), compound (5), compound (6), compound (7), compound (8), compound (9), compound (10), compound (11), compound (12), compound (13), compound (25), compound (27), or compound (28)

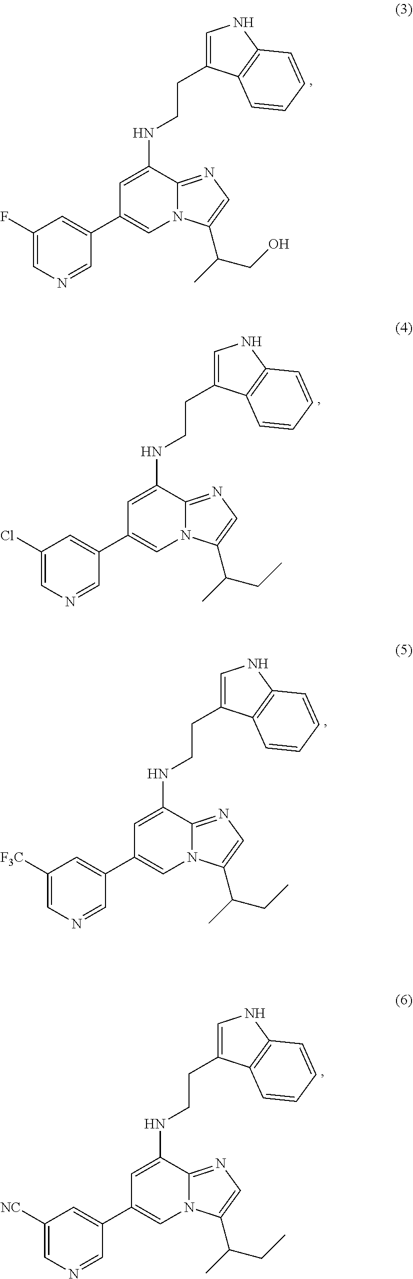 [SMILES: CC(CO)c1cnc2c(NCCc3c[nH]c4ccccc34)cc(-c3cncc(F)c3)cn12 ; CCC(C)c1cnc2c(NCCc3c[nH]c4ccccc34)cc(-c3cncc(Cl)c3)cn12 ; CCC(C)c1cnc2c(NCCc3c[nH]c4ccccc34)cc(-c3cncc(C(F)(F)F)c3)cn12 ; CCC(C)c1cnc2c(NCCc3c[nH]c4ccccc34)cc(-c3cncc(C#N)c3)cn12]

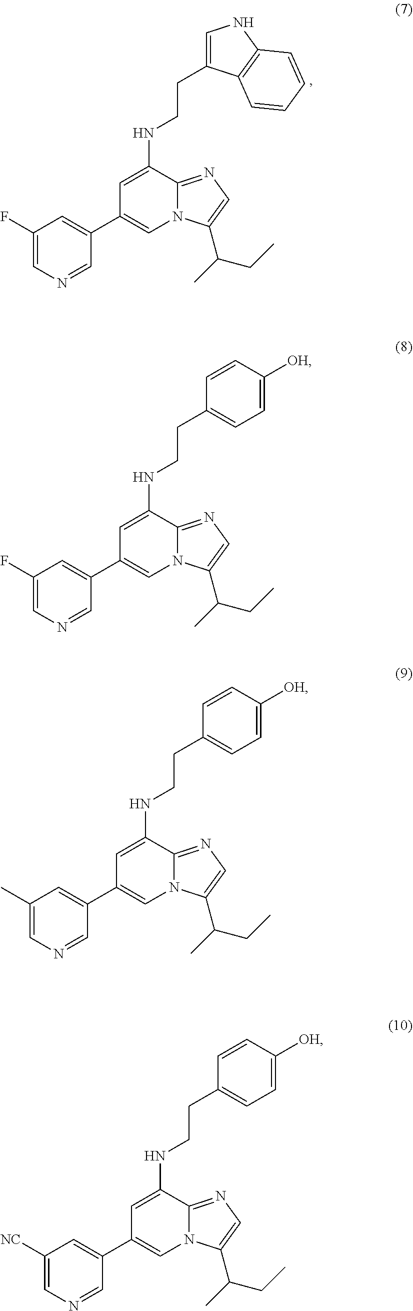 [SMILES: CCC(C)c1cnc2c(NCCc3c[nH]c4ccccc34)cc(-c3cncc(F)c3)cn12 ; CCC(C)c1cnc2c(NCCc3ccc(O)cc3)cc(-c3cncc(F)c3)cn12 ; CCC(C)c1cnc2c(NCCc3ccc(O)cc3)cc(-c3cncc(C)c3)cn12 ; CCC(C)c1cnc2c(NCCc3ccc(O)cc3)cc(-c3cncc(C#N)c3)cn12]

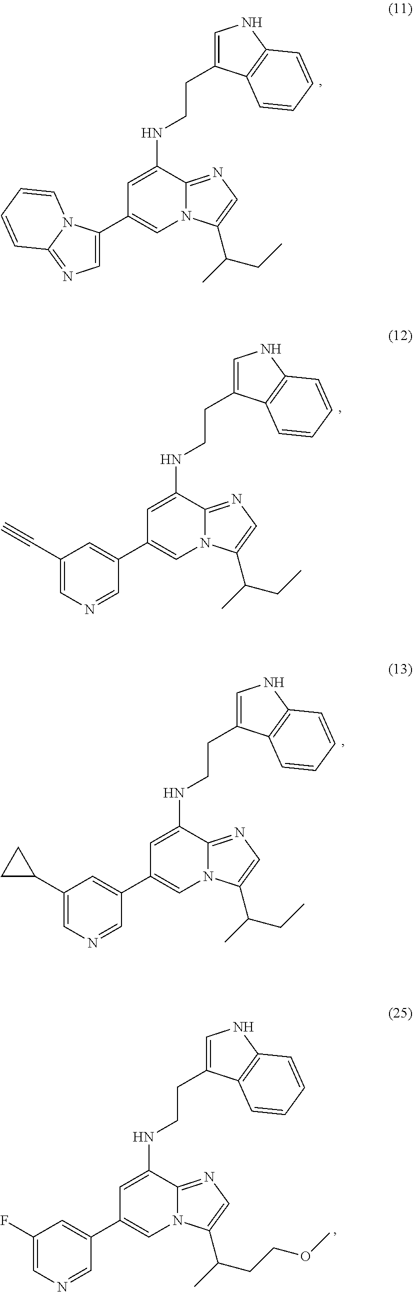 [SMILES: CCC(C)c1cnc2c(NCCc3c[nH]c4ccccc34)cc(-c3cnc4ccccn34)cn12 ; C#Cc1cncc(-c2cc(NCCc3c[nH]c4ccccc34)c3ncc(C(C)CC)n3c2)c1 ; CCC(C)c1cnc2c(NCCc3c[nH]c4ccccc34)cc(-c3cncc(C4CC4)c3)cn12 ; COCCC(C)c1cnc2c(NCCc3c[nH]c4ccccc34)cc(-c3cncc(F)c3)cn12]

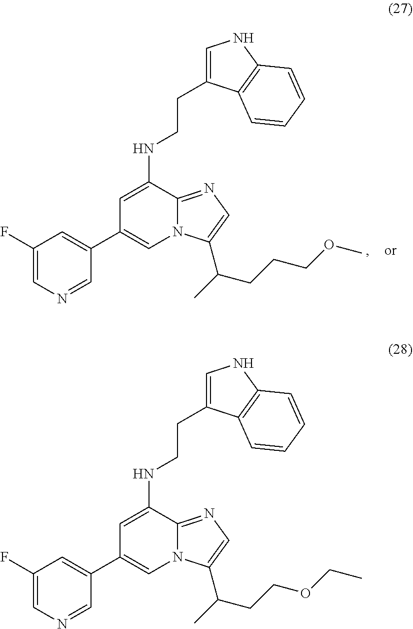 [SMILES: COCCCC(C)c1cnc2c(NCCc3c[nH]c4ccccc34)cc(-c3cncc(F)c3)cn12 ; CCOCCC(C)c1cnc2c(NCCc3c[nH]c4ccccc34)cc(-c3cncc(F)c3)cn12]

or a pharmaceutically acceptable salt, hydrate, or solvate thereof.

In some embodiments, the aryl hydrocarbon receptor antagonist is a compound represented by formula (V)

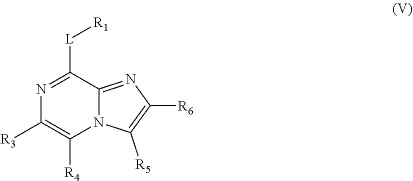

or a pharmaceutically acceptable salt, hydrate, or solvate thereof, wherein:

L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—. —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, —NR₇ₐ(CR₈ₐR_{8b})ₙNR₇ₐ—, —NR₇ₐ(CR₈ₐR_{8b})ₙO—, —NR₇ₐ(CR₈ₐR_{8b})ₙS—, —O(CR₈ₐR_{8b})ₙNR₇ₐ—, —O(CR₈ₐR_{8b})ₙO—, —O(CR₈ₐR_{8b})ₙS—, —S(CR₈ₐR_{8b})ₙNR₇ₐ—, —S(CR₈ₐR_{8b})ₙO—, —S(CR₈ₐR_{8b})ₙS—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, —OC(S)NR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, —OC(S)CR₉ₐR_{9b}R_{9c}, phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}; wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, and —OC(S)CR₉ₐR_{9b}R_{9c}.

In some embodiments, R₁ is selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}.

In some embodiments, R₁ is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, R₁ is selected from the group consisting of:

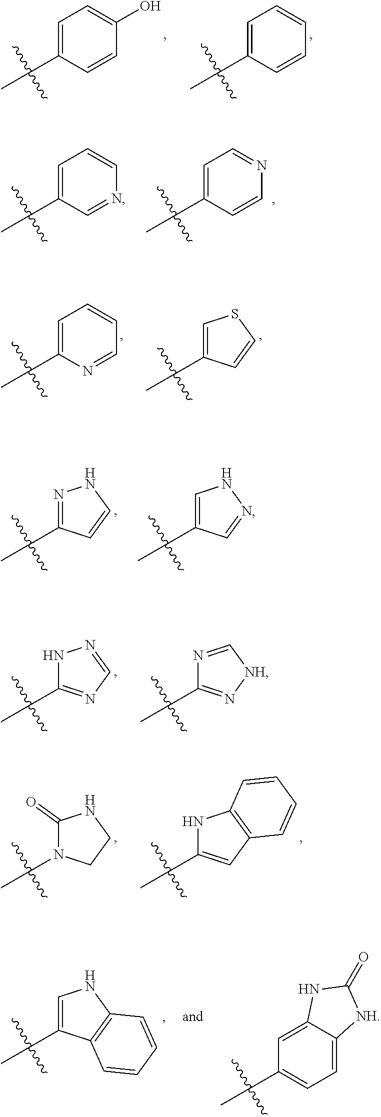

In some embodiments, R₁ is selected from the group consisting of:

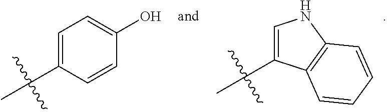 [SMILES: *c1ccc(O)cc1 ; *c1c[nH]c2ccccc12]

In some embodiments, R₁ is selected from the group consisting of phenol-4-yl and 1H-indol-3-yl.

In some embodiments, L is selected from the group consisting of —NR₇ₐ(CR₈ₐR_{8b})ₙ— and —O(CR₈ₐR_{8b})ₙ—.

In some embodiments, L is selected from the group consisting of —NH(CH₂)₂— and —O(CH₂)₂—.

In some embodiments, R₃ is selected from the group consisting of optionally substituted aryl and optionally substituted heteroaryl.

In some embodiments, R₃ is selected from the group consisting of phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the phenyl, thiophenyl, furanyl, 1H-benzoimidazolyl, quinolinyl, isoquinolinyl, imidazopyridinyl, benzothiophenyl, pyrimidinyl, pyridinyl, 1H-imidazolyl, pyrazinyl, pyridazinyl, 1H-pyrrolyl, or thiazolyl is optionally substituted, for example, with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, and wherein R₁₁ₐ and R_{11b} are each independently selected from the group consisting of hydrogen and C₁₋₄ alkyl.

In some embodiments, R₃ is selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted, for example, with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, and imidazo[1,2-a]pyridin-3-yl, wherein the thiophen-3-yl, benzo[b]thiophen-3-yl, pyridin-3-yl, pyrimidin-5-yl, 1H-imidazol-1-yl, 1H-benzo[d]imidazol-1-yl, isoquinolin-4-yl, 1H-imidazo[4,5-b]pyridin-1-yl, or imidazo[1,2-a]pyridin-3-yl is optionally substituted, for example, with from 1 to 3 substituents independently selected from the group consisting of cyano, hydroxy. C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}.

In some embodiments, R₃ is selected from the group consisting of optionally substituted:

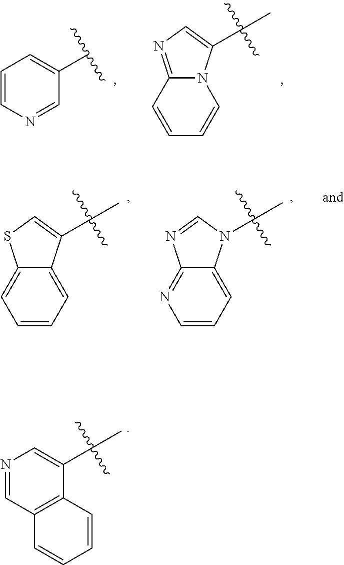 [SMILES: *c1cccnc1 ; *c1cnc2ccccn12 ; *c1csc2ccccc12 ; *n1cnc2ncccc21 ; *c1cncc2ccccc12]

In some embodiments, R₃ is pyridin-3-yl, wherein the pyridin-3-yl is optionally substituted at C5, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, the pyridin-3-yl is substituted at C5 with a substituent selected from the group consisting of ethoxycarbonyl, methoxy, cyano, methyl, methylsulfonyl, fluoro, chloro, trifluoromethyl, ethynyl, and cyclopropyl.

In some embodiments, R₃ is selected from the group consisting of:

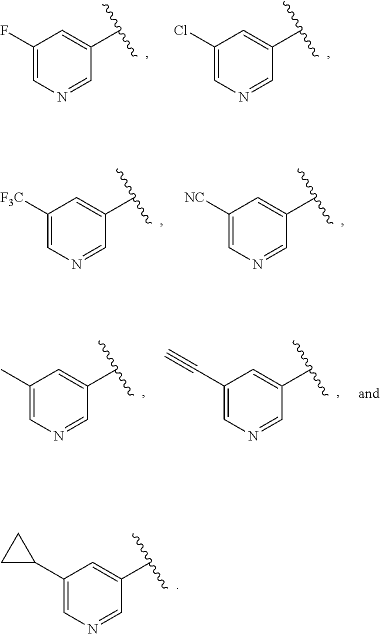

In some embodiments, R₃ is imidazo[1,2-a]pyridin-3-yl, wherein the imidazo[1,2-a]pyridin-3-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₃ is benzo[b]thiophen-3-yl, wherein the benzo[b]thiophen-3-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁ₐ, —C(O)OR₁₁ₐ, and —C(O)NR₁₁ₐR_{11b}.

In some embodiments, R₃ is 1H-imidazo[4,5-b]pyridin-1-yl, wherein the 1H-imidazo[4,5-b]pyridin-1-yl is optionally substituted, for example, with a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁ₐ, —C(O)OR₁₁ₐ, and —C(O)NR₁₁ₐR_{11b}.

In some embodiments, R₃ is isoquinolin-4-yl, wherein the isoquinolin-4-yl is optionally substituted, for example, with a substituent selected from the group consisting of C1-4 alkyl, halo, halo-substituted-C1-4 alkyl, C2-4 alkenyl, C2-4 alkynyl, C3-8 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}.

In some embodiments, R₄ is hydrogen.

In some embodiments, R₃ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted, for example, with from 1 to 3 substituents independently selected from the group consisting of hydroxy, C1-4 alkyl, and halo-substituted-C1-4 alkyl.

In some embodiments, R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl.

In some embodiments, R₅ is (S)-1-hydroxypropan-2-yl.

In some embodiments, R₅ is (R)-1-hydroxypropan-2-yl.

In some embodiments, R₅ is (S)-sec-butyl.

In some embodiments, R₅ is (R)-sec-butyl.

In some embodiments, R₃ is selected from the group consisting of (i), (ii), (iii), (iv), and (v)

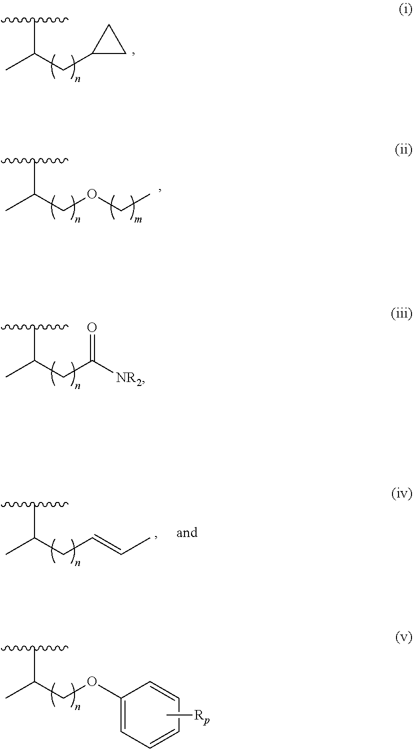

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

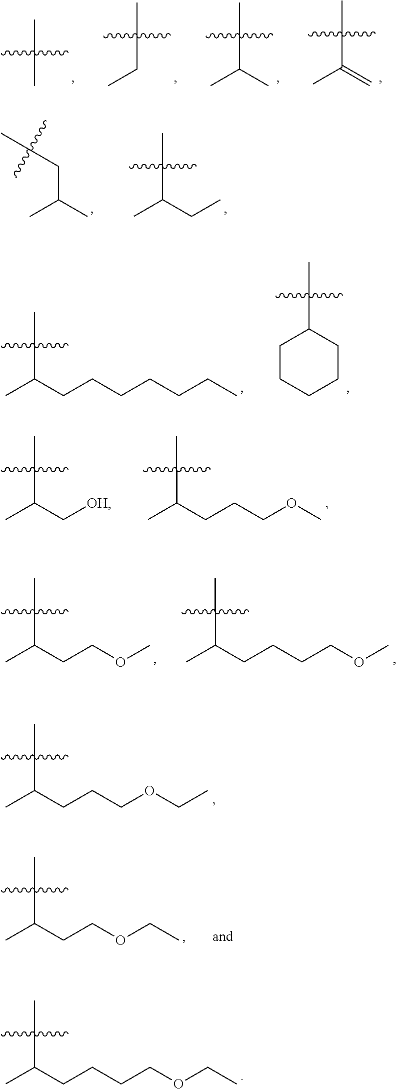

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, R₅ is (S)-4-methoxybutan-2-yl.

In some embodiments, R₅ is (R)-4-methoxybutan-2-yl.

In some embodiments, R₅ is (S)-5-methoxypentan-2-yl.

In some embodiments, R₅ is (R)-5-methoxypentan-2-yl.

In some embodiments, R₅ is (S)-4-ethoxybutan-2-yl.

In some embodiments, R₅ is (R)-4-ethoxybutan-2-yl.

In some embodiments, R₈ is hydrogen.

In some embodiments, the aryl hydrocarbon receptor antagonist is a compound represented by formula (V-a)

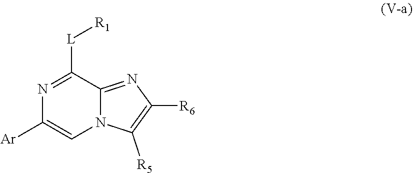

or a pharmaceutically acceptable salt, hydrate, or solvate thereof, wherein:

L is a linker selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ, and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R₉ₐ, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl (for example, R₁ may be selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted, for example, with from 1 to 3 substituents independently selected from the group consisting of cyano, hydroxy, C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl);

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, Ar is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (V-b)

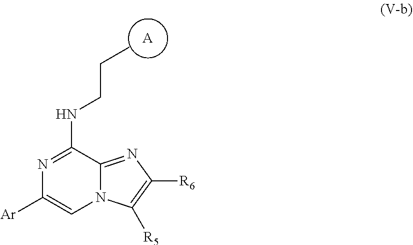

or a pharmaceutically acceptable salt, hydrate, or solvate thereof, wherein:

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

Ar is selected from the group consisting of optionally substituted monocyclic aryl and heteroaryl, such as optionally substituted thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, A is selected from the group consisting of phenyl, phenol-4-yl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl.

In some embodiments, A is selected from the group consisting of phenol-4-yl and 1H-indol-3-yl.

In some embodiments, the aryl hydrocarbon receptor antagonist is a compound represented by formula (V-c)

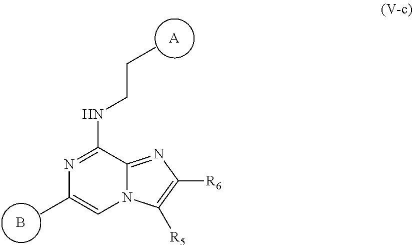

or a pharmaceutically acceptable salt, hydrate, or solvate thereof, wherein

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR_{11b}R_{11b}, wherein R₁₁ₐ and R_{11b} are each independently selected from the group consisting of hydrogen and C₁₋₄ alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

In some embodiments, B is pyridin-3-yl, wherein the pyridin-3-yl is optionally substituted at C5, for example, with a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (V-d)

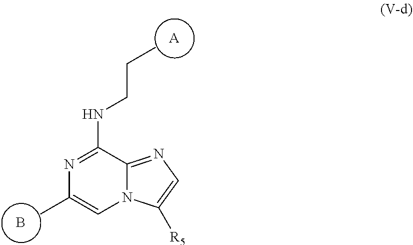

or a pharmaceutically acceptable salt, hydrate, or solvate thereof, wherein:

A is an optionally substituted ring system selected from the group consisting of phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, and 1H-indazolyl, wherein the phenyl, 1H-pyrrolopyridinyl, 1H-indolyl, thiophenyl, pyridinyl, 1H-1,2,4-triazolyl, 2-oxoimidazolidinyl, 1H-pyrazolyl, 2-oxo-2,3-dihydro-1H-benzoimidazolyl, or 1H-indazolyl is optionally substituted with from 1 to 3 substituents independently selected from the group consisting of cyano, hydroxy, C1-4 alkyl, C₁₋₄ alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{11b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophenyl, furanyl, 1H-benzoimidazolyl, isoquinolinyl, imidazopyridinyl, benzothiophenyl, pyrimidinyl, pyridinyl, 1H-imidazolyl, pyrazinyl, pyridazinyl, 1H-pyrrolyl, and thiazolyl, wherein the thiophenyl, furanyl, 1H-benzoimidazolyl, isoquinolinyl, 1H-imidazopyridinyl, benzothiophenyl, pyrimidinyl, pyridinyl, 1H-imidazolyl, pyrazinyl, pyridazinyl, 1H-pyrrolyl, or thiazolyl is optionally substituted with from 1 to 3 substituents independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl.

In some embodiments, the aryl hydrocarbon receptor antagonist is a compound represented by formula (V-e)

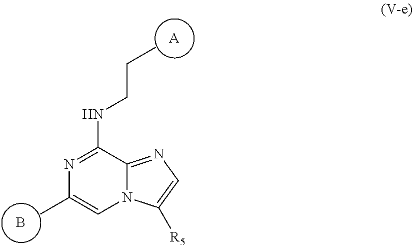

or a pharmaceutically acceptable salt, hydrate, or solvate thereof, wherein:

A is an optionally substituted ring system selected from the group consisting of phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, and 2-oxo-2,3-dihydro-1H-benzo[d]imidazol-5-yl, wherein the phenyl, 1H-indol-2-yl, 1H-indol-3-yl, thiophen-3-yl, pyridin-2-yl, pyridin-3-yl, pyridin-4-yl, 1H-1,2,4-triazol-3-yl, 1H-1,2,4-triazol-5-yl, 2-oxoimidazolidin-1-yl, 1H-pyrazol-3-yl, 1H-pyrazol-4-yl, or 2-oxo-2,3-dihydro-1H-benzo[d]imidazol-5-yl is optionally substituted with from 1 to 3 substituents independently selected from the group consisting of cyano, hydroxy. C1-4 alkyl, C1-4 alkoxy, halo, halo-substituted-C1-4 alkyl, halo-substituted-C1-4 alkoxy, amino, —O(CH₂)₂NR₁₀ₐR_{10b}, —S(O)₂NR₁₀ₐR_{10b}, —OS(O)₂NR₁₀ₐR_{10b}, and —NR₁₀ₐS(O)₂R_{10b}, wherein R₁₀ₐ and R_{10b} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

B is an optionally substituted ring system selected from the group consisting of thiophen-2-yl, thiophen-3-yl, furan-3-yl, 1H-benzo[d]imidazol-1-yl, isoquinolin-4-yl, 1H-imidazo[4,5-b]pyridin-1-yl, imidazo[1,2-a]pyridin-3-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl and thiazol-5-yl, wherein the thiophen-2-yl, thiophen-3-yl, furan-3-yl, 1H-benzo[d]imidazol-1-yl, isoquinolin-4-yl, 1H-imidazo[4,5-b]pyridin-1-yl, benzo[b]thiophen-3-yl, pyrimidin-5-yl, pyridin-2-yl, pyridin-3-yl, pyridin-4-yl, 1H-imidazol-1-yl, pyrazin-2-yl, pyridazin-4-yl, 1H-pyrrol-2-yl, or thiazol-5-yl is optionally substituted with from 1 to 3 substituents independently selected from the group consisting of cyano, hydroxy. C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₁ₐ, —S(O)₀₋₂R_{11b}, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₃ is selected from the group consisting of (i), (i), (iii), (iv), and (v)

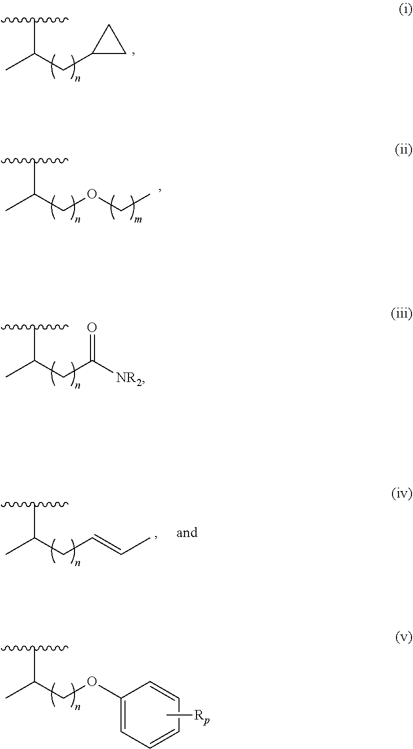

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₃ is selected from the group consisting of:

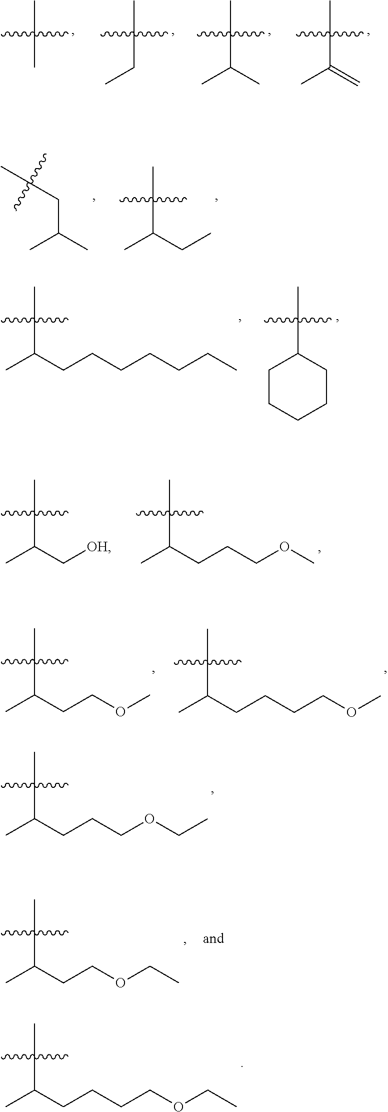

In some embodiments, R₅ is (ii).

In some embodiments, R₃ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-8-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (V-f)

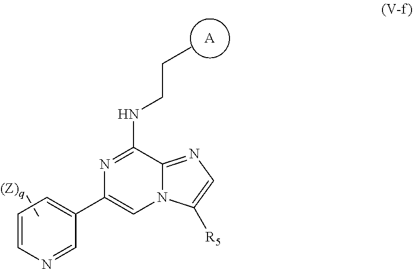

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4;

each Z is independently a substituent selected from the group consisting of C1-4 alkyl, halo, halo-substituted-C1-4 alkyl, C2-4 alkenyl, C2-4 alkynyl, C3-8 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₃ is selected from the group consisting of (i), (ii), (iii), (iv), and (v)

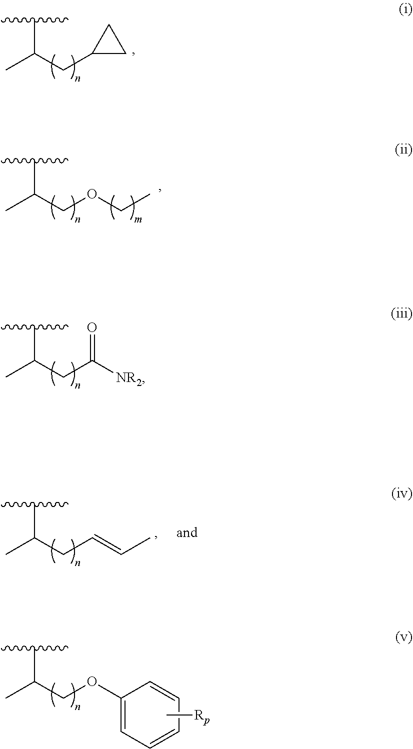

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

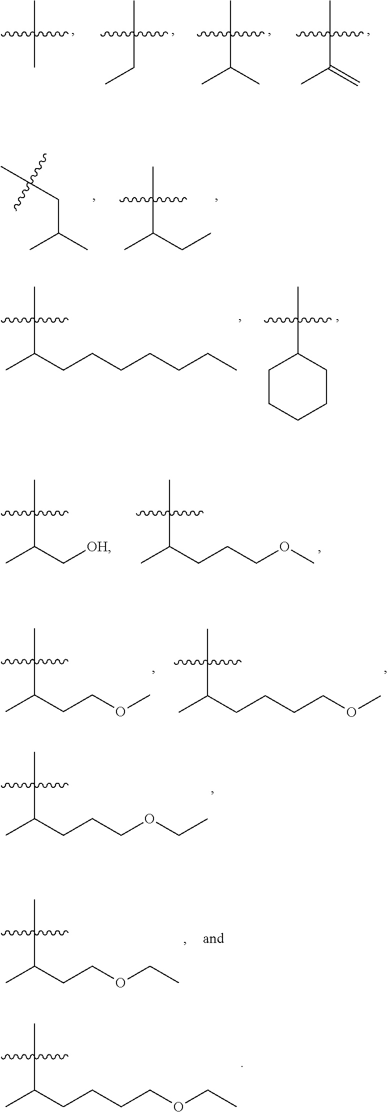

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, each Z is independently a substituent selected from the group consisting of ethoxycarbonyl, methoxy, cyano, methyl, methylsulfonyl, fluoro, chloro, trifluoromethyl, ethynyl, and cyclopropyl.

In some embodiments, the aryl hydrocarbon receptor antagonist is a compound represented by formula (V-g)

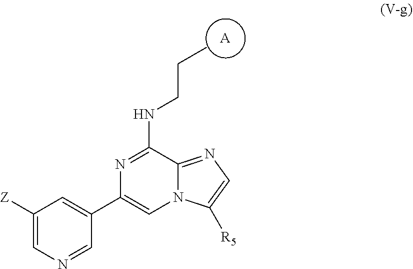

or a pharmaceutically acceptable salt, hydrate, or solvate thereof, wherein:

A is an optionally substituted ring system selected from the group consisting of phenol-4-yl and 1H-indol-3-yl;

Z is a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ and —C(O)NR₁₁ₐR_{11b}, wherein R₁₁ₐ and R_{11b} are each independently selected from the group consisting of hydrogen and C₁₋₄ alkyl; and

R₅ is selected from the group consisting of isopropyl, methyl, ethyl, prop-1-en-2-yl, isobutyl, cyclohexyl, sec-butyl, (S)-sec-butyl, (R)-sec-butyl, 1-hydroxypropan-2-yl, (S)-1-hydroxypropan-2-yl, (R)-1-hydroxypropan-2-yl, and nonan-2-yl, or R₃ is selected from the group consisting of (i), (i), (iii), (iv), and (v)

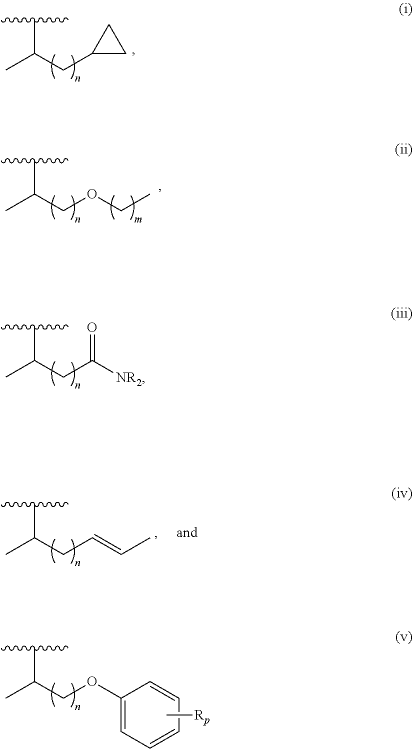

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₃ is selected from the group consisting of:

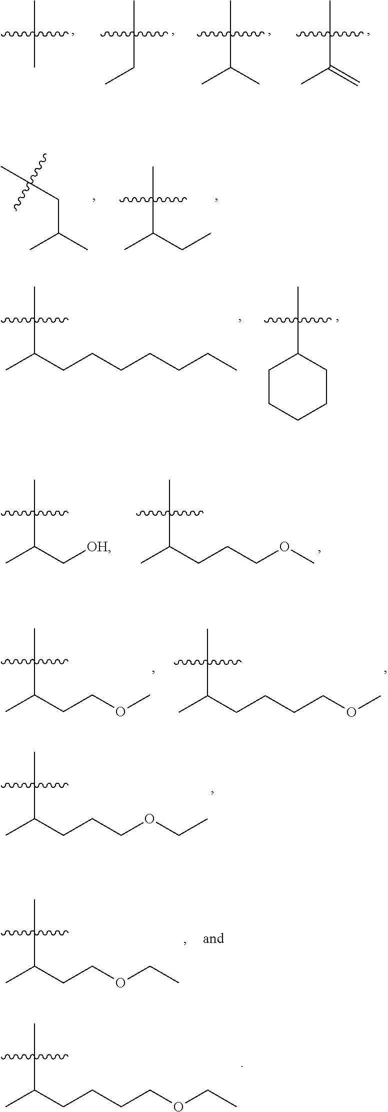

in some embodiments, R₅ is (ii).

in some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (V-h)

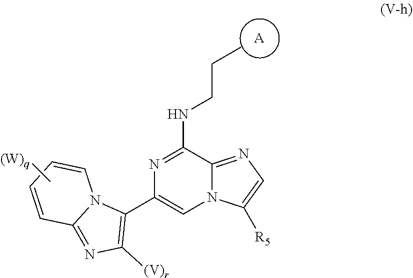

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4:

r is 0 or 1;

W and V are each independently a substituent selected from the group consisting of C1-4 alkyl, halo, halo-substituted-C1-4 alkyl. C2-4 alkenyl, C2-4 alkynyl. C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₃ is selected from the group consisting of (i), (ii), (iii), (iv), and (v)

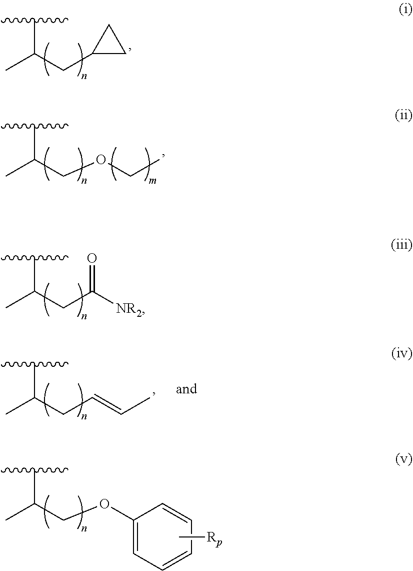

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl. C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₃ is selected from the group consisting of:

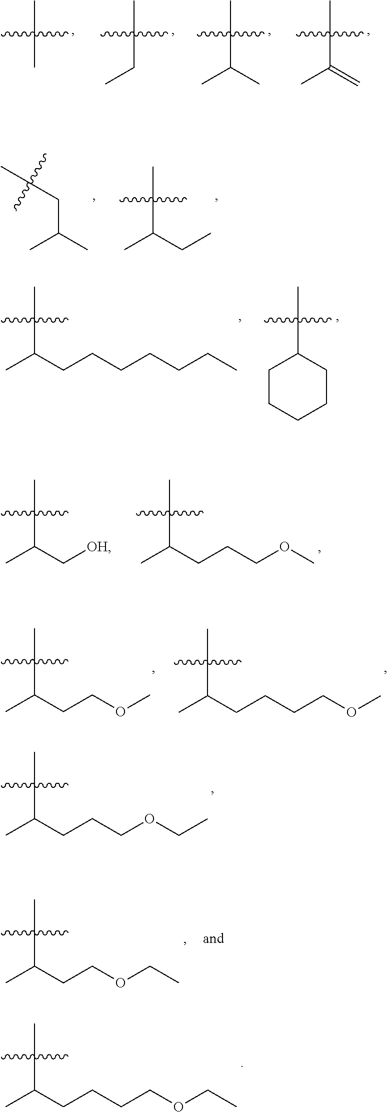

In some embodiments, R₅ is (ii).

In some embodiments, R₃ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-8-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (V-i)

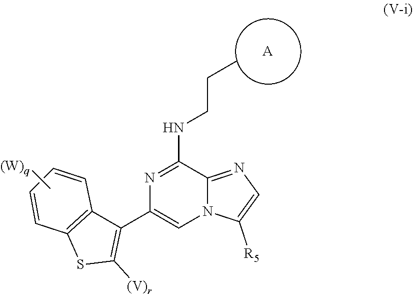

or a pharmaceutically acceptable salt, hydrate, or solvate thereof, wherein

A is an optionally substituted ring system selected from the group consisting of phenol-4-yl and 1H-indol-3-yl;

q is an integer from 0 to 4:

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₃ is selected from the group consisting of (i), (ii), (iii), (iv), and (v)

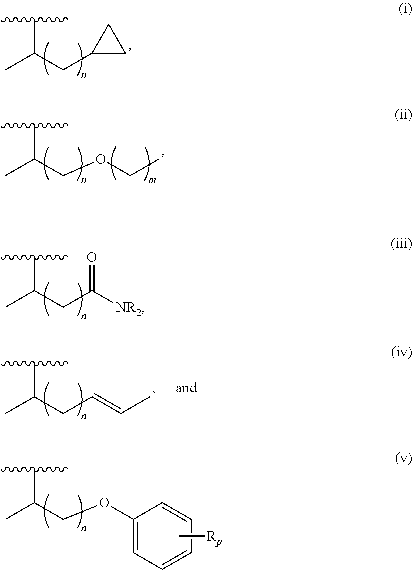

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

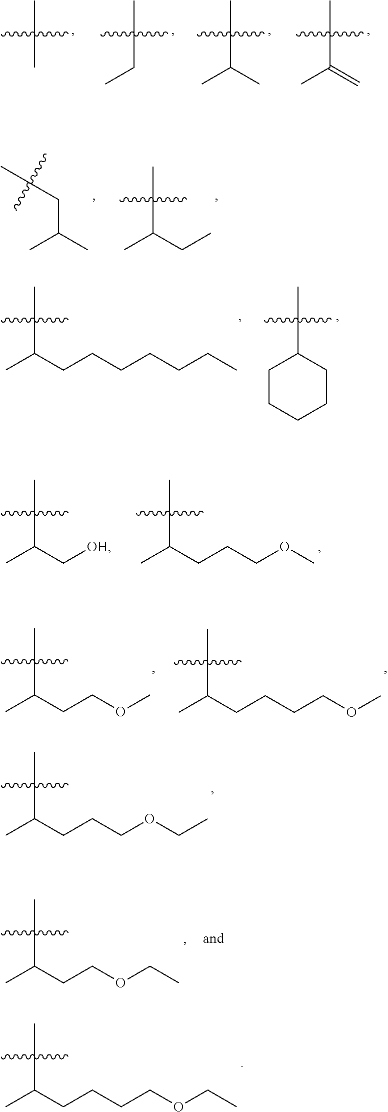

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (V-j)

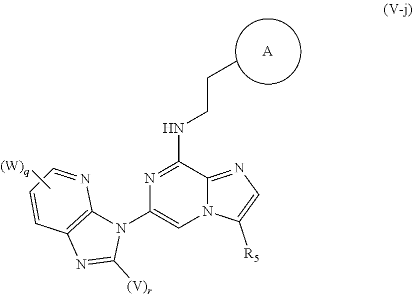

or a pharmaceutically acceptable salt, hydrate, or solvate thereof, wherein:

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-8,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₅ is selected from the group consisting of (i), (i), (iii), (iv), and (v)

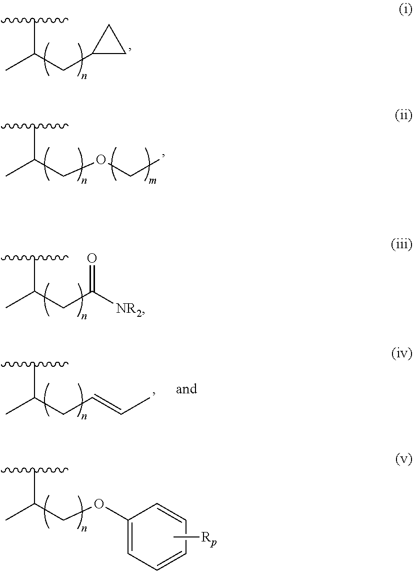

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₃ is selected from the group consisting of:

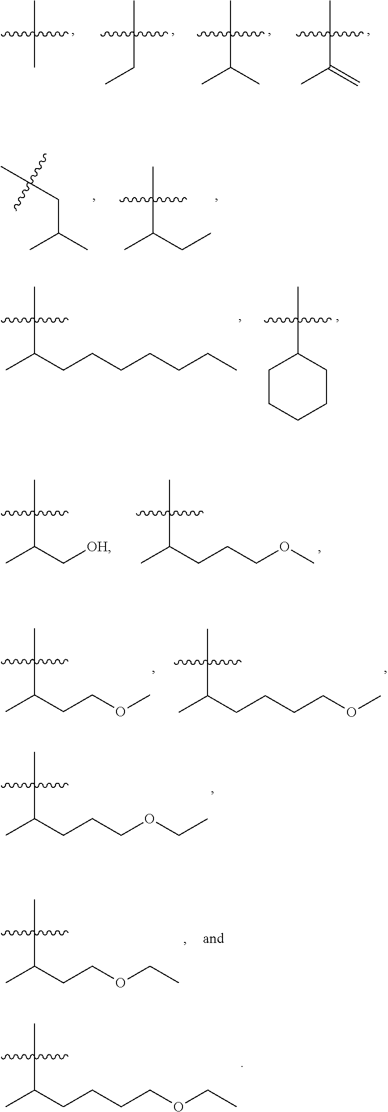

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is a compound represented by formula (V-k)

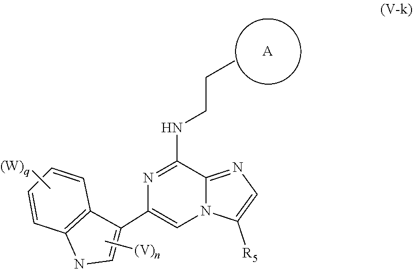

or a pharmaceutically acceptable salt, hydrate, or solvate thereof, wherein

A is an optionally substituted ring system selected from the group consisting of phenol-4-yl and 1H-indol-3-yl;

q is an integer from 0 to 4;

r is 0 or 1;

W and V are each independently a substituent selected from the group consisting of C1-4 alkyl, halo, halo-substituted-C1-4 alkyl, C2-4 alkenyl, C2-4 alkynyl, C3-6 cycloalkyl, C1-4 alkoxy, cyano, amino, C(O)R₁₁ₐ, —S(O)₀₋₂R₁₁ₐ, —C(O)OR₁₁ₐ, and —C(O)NR₁₁ₐR_{11b}, wherein R₁₁ₐ and R_{11b} are each independently selected from the group consisting of hydrogen and C₁₋₄ alkyl; and

R₅ is selected from the group consisting of C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, and 1-(1-(2-oxo-6,9,12-trioxa-3-azatetradecan-14-yl)-1H-1,2,3-triazol-4-yl)ethyl, wherein the C1-10 alkyl, prop-1-en-2-yl, cyclohexyl, cyclopropyl, 2-(2-oxopyrrolidin-1-yl)ethyl, oxetan-2-yl, oxetan-3-yl, benzhydryl, tetrahydro-2H-pyran-2-yl, tetrahydro-2H-pyran-3-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl is optionally substituted with from 1 to 3 substituents independently selected from the group consisting of hydroxy, C1-4 alkyl, and halo-substituted-C1-4 alkyl, or R₅ is selected from the group consisting of (i), (ii), (iii), (iv), and (v)

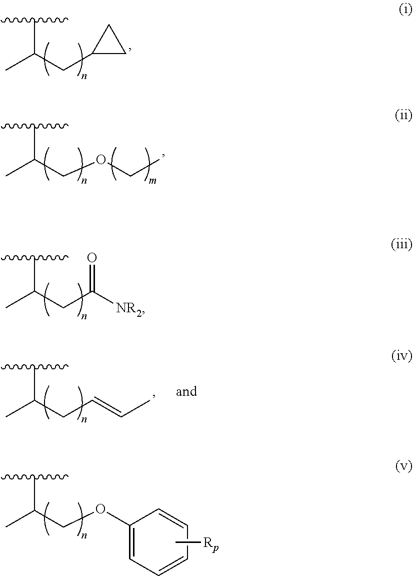

wherein n is an integer from 1 to 6, m is an integer from 0 to 6, p is an integer from 0 to 5, and each R is independently selected from the group consisting of cyano, hydroxy, C1-4 alkyl, C2-4 alkenyl, C2-4 alkynyl, C3-6 cycloalkyl, C1-4 alkoxy, halo, halo-substituted-C1-4 alkyl, halo-substituted-C1-4 alkoxy, amino, —C(O)R₁₂ₐ, —S(O)₀₋₂R₁₂ₐ, —C(O)OR₁₂ₐ, and —C(O)NR₁₂ₐR_{12b}, and wherein R₁₂ₐ and R_{12b} are each independently selected from the group consisting of hydrogen and C₁₋₄ alkyl.

In some embodiments, R₅ is selected from the group consisting of:

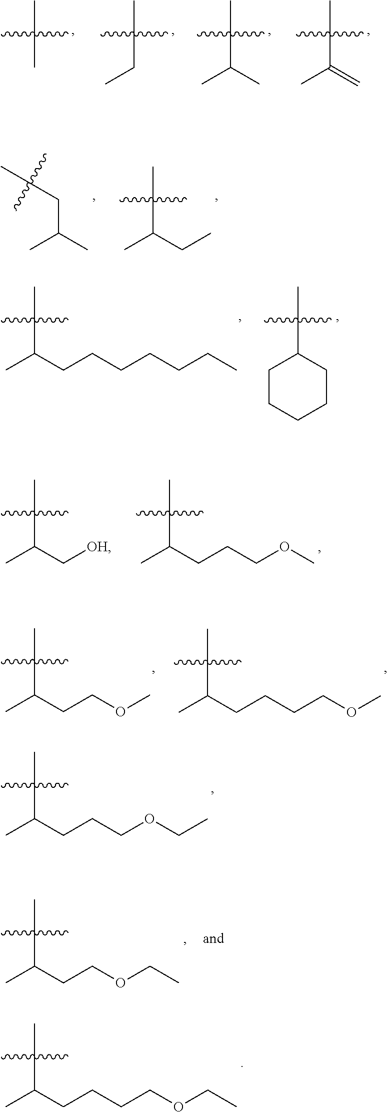

In some embodiments, R₅ is (ii).

In some embodiments, R₅ is selected from the group consisting of 4-methoxybutan-2-yl, (S)-4-methoxybutan-2-yl, (R)-4-methoxybutan-2-yl, 4-ethoxybutan-2-yl, (S)-4-ethoxybutan-2-yl, (R)-4-ethoxybutan-2-yl, 5-methoxypentan-2-yl, (S)-5-methoxypentan-2-yl, (R)-5-methoxypentan-2-yl, 5-ethoxypentan-2-yl, (S)-5-ethoxypentan-2-yl, (R)-5-ethoxypentan-2-yl, 6-methoxyhexan-2-yl, (S)-6-methoxyhexan-2-yl, (R)-6-methoxyhexan-2-yl, 6-ethoxyhexan-2-yl, (S)-6-ethoxyhexan-2-yl, and (R)-6-ethoxyhexan-2-yl.

In some embodiments, the aryl hydrocarbon receptor antagonist is compound (14), compound (15), compound (16), compound (17), compound (18), compound (19), compound (20), compound (21), compound (22), compound (23), compound (24), compound (26), compound (29), or compound (30)

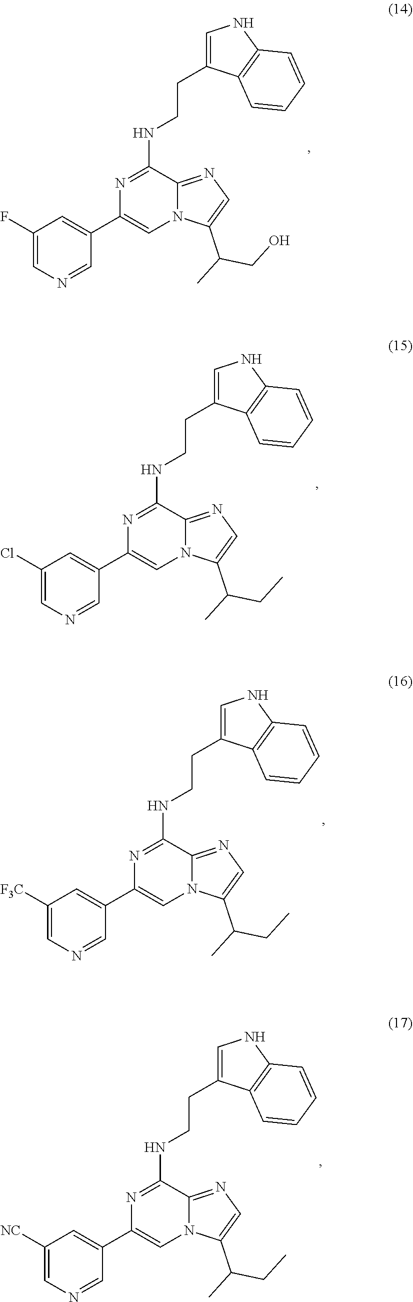 [SMILES: CC(CO)c1cnc2c(NCCc3c[nH]c4ccccc34)nc(-c3cncc(F)c3)cn12 ; CCC(C)c1cnc2c(NCCc3c[nH]c4ccccc34)nc(-c3cncc(Cl)c3)cn12 ; CCC(C)c1cnc2c(NCCc3c[nH]c4ccccc34)nc(-c3cncc(C(F)(F)F)c3)cn12 ; CCC(C)c1cnc2c(NCCc3c[nH]c4ccccc34)nc(-c3cncc(C#N)c3)cn12]

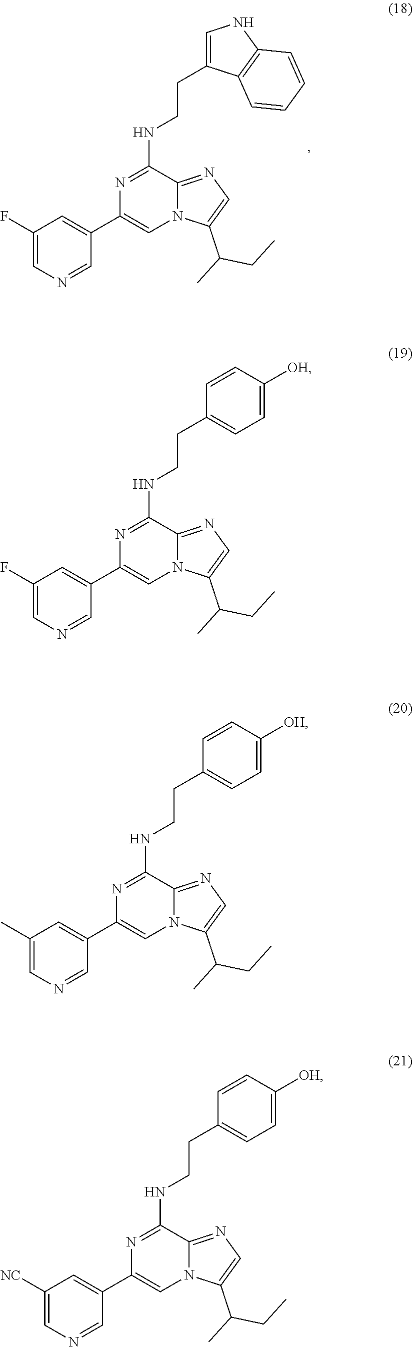 [SMILES: CCC(C)c1cnc2c(NCCc3c[nH]c4ccccc34)nc(-c3cncc(F)c3)cn12 ; CCC(C)c1cnc2c(NCCc3ccc(O)cc3)nc(-c3cncc(F)c3)cn12 ; CCC(C)c1cnc2c(NCCc3ccc(O)cc3)nc(-c3cncc(C)c3)cn12 ; CCC(C)c1cnc2c(NCCc3ccc(O)cc3)nc(-c3cncc(C#N)c3)cn12]

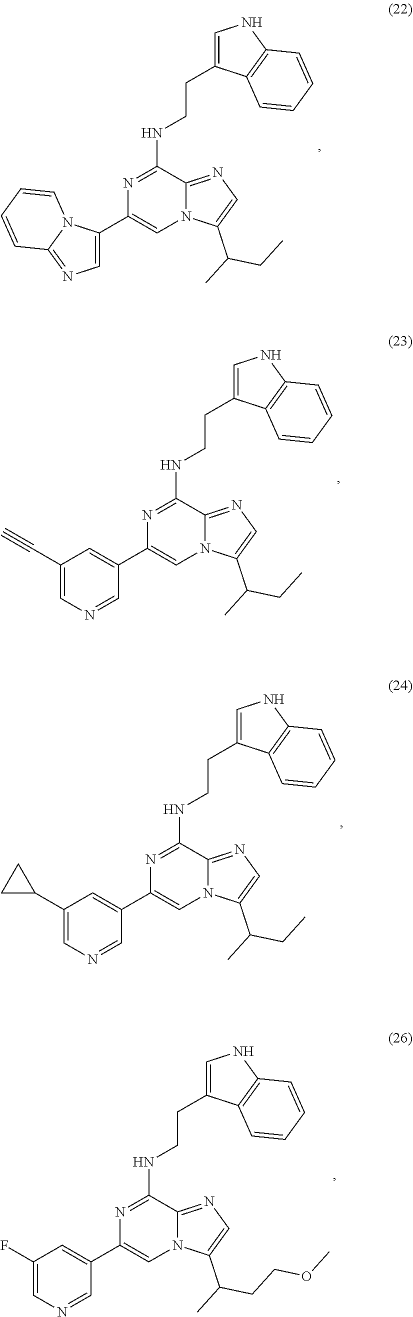 [SMILES: CCC(C)c1cnc2c(NCCc3c[nH]c4ccccc34)nc(-c3cnc4ccccn34)cn12 ; C#Cc1cncc(-c2cn3c(C(C)CC)cnc3c(NCCc3c[nH]c4ccccc34)n2)c1 ; CCC(C)c1cnc2c(NCCc3c[nH]c4ccccc34)nc(-c3cncc(C4CC4)c3)cn12 ; COCCC(C)c1cnc2c(NCCc3c[nH]c4ccccc34)nc(-c3cncc(F)c3)cn12]

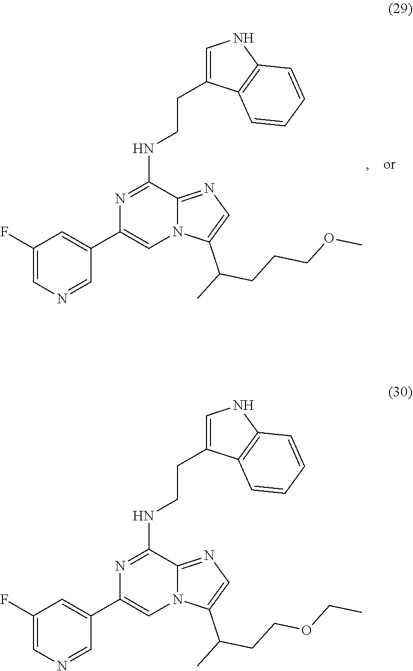 [SMILES: COCCCC(C)c1cnc2c(NCCc3c[nH]c4ccccc34)nc(-c3cncc(F)c3)cn12 ; CCOCCC(C)c1cnc2c(NCCc3c[nH]c4ccccc34)nc(-c3cncc(F)c3)cn12]

or a pharmaceutically acceptable salt, hydrate, or solvate thereof.

### CXCR4 Antagonists

Exemplary CXCR4 antagonists for use in conjunction with the compositions and methods described herein are compounds represented by formula (I)

Z-linker-Z′ (I)

or a pharmaceutically acceptable salt thereof, wherein Z is:
(i) a cyclic polyamine containing from 9 to 32 ring members, wherein from 2 to 8 of the ring members are nitrogen atoms separated from one another by 2 or more carbon atoms; or
(ii) an amine represented by formula (IA)

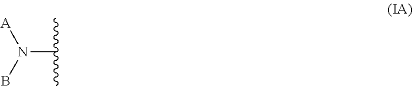

wherein A includes a monocyclic or bicyclic fused ring system including at least one nitrogen atom and B is H or a substituent of from 1 to 20 atoms;
and wherein Z′ is:
(i) a cyclic polyamine containing from 9 to 32 ring members, wherein from 2 to 8 of the ring members are nitrogen atoms separated from one another by 2 or more carbon atoms;
(ii) an amine represented by formula (IB)

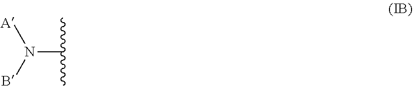

wherein A′ includes a monocyclic or bicyclic fused ring system including at least one nitrogen atom and B′ is H or a substituent of from 1 to 20 atoms; or
(iii) a substituent represented by formula (IC)

N(R)—(CR₂)ₙ—X (IC)

wherein each R is independently H or C₁-C₆ alkyl, n is 1 or 2, and X is an aryl or heteroaryl group or a mercaptan;

wherein the linker is a bond, optionally substituted alkylene (e.g., optionally substituted C₁-C₆ alkylene), optionally substituted heteroalkylene (e.g., optionally substituted C₁-C₆ heteroalkylene), optionally substituted alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), optionally substituted heteroalkynylene (e.g., optionally substituted C₂-C₆ heteroalkynylene), optionally substituted cycloalkylene, optionally substituted heterocycloalkylene, optionally substituted arylene, or optionally substituted heteroarylene.

In some embodiments, Z and Z′ may each independently a cyclic polyamine containing from 9 to 32 ring members, of which from 2 to 8 are nitrogen atoms separated from one another by 2 or more carbon atoms. In some embodiments, Z and Z′ are identical substituents. As an example, Z may be a cyclic polyamine including from 10 to 24 ring members. In some embodiments, Z may be a cyclic polyamine that contains 14 ring members. In some embodiments, Z includes 4 nitrogen atoms. In some embodiments, Z is 1,4,8,11-tetraazocyclotetradecane.

In some embodiments, the linker is represented by formula

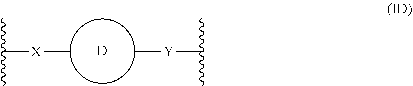

wherein ring D is an optionally substituted aryl group, an optionally substituted heteroaryl group, an optionally substituted cycloalkyl group, or an optionally substituted heterocycloalkyl group; and

X and Y are each independently optionally substituted alkylene (e.g., optionally substituted C₁-C₆ alkylene), optionally substituted heteroalkylene (e.g., optionally substituted C₁-C₆ heteroalkylene), optionally substituted alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), or optionally substituted heteroalkynylene (e.g., optionally substituted C₂-C₆ heteroalkynylene).

As an example, the linker may be represented by formula (IE)

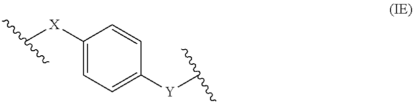

wherein ring D is an optionally substituted aryl group, an optionally substituted heteroaryl group, an optionally substituted cycloalkyl group, or an optionally substituted heterocycloalkyl group; and

X and Y are each independently optionally substituted alkylene (e.g., optionally substituted C₁-C₆ alkylene), optionally substituted heteroalkylene (e.g., optionally substituted C₁-C₆ heteroalkylene), optionally substituted C₂-C₆ alkenylene (e.g., optionally substituted C₂-C₆ alkenylene), optionally substituted heteroalkenylene (e.g., optionally substituted C₂-C₆ heteroalkenylene), optionally substituted alkynylene (e.g., optionally substituted C₂-C₆ alkynylene), or optionally substituted heteroalkynylene (e.g., optionally substituted C₂-C₆ heteroalkynylene). In some embodiments. X and Y are each independently optionally substituted C₁-C₆ alkylene. In some embodiments, X and Y are identical substituents. In some embodiments, X and Y may be each be methylene, ethylene, n-propylene, n-butylene, n-pentylene, or n-hexylene groups. In some embodiments, X and Y are each methylene groups.

The linker may be, for example, 1,3-phenylene, 2,6-pyridine, 3,5-pyridine, 2,5-thiophene, 4,4′-(2,2′-bipyrimidine), 2,9-(1,10-phenanthroline), or the like. In some embodiments, the linker is 1,4-phenylene-bis-(methylene).

CXCR4 antagonists useful in conjunction with the compositions and methods described herein include plerixafor (also referred to herein as “AMD3100” and “Mozibil”), or a pharmaceutically acceptable salt thereof, represented by formula (II), 1,1′-[1,4-phenylenebis(methylene)]-bis-1,4,8,11-tetra-azacyclotetradecane.

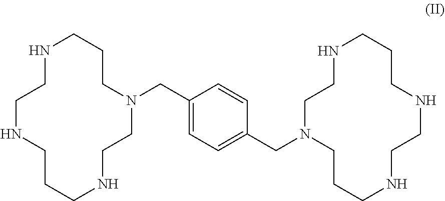 [SMILES: c1cc(CN2CCCNCCNCCCNCC2)ccc1CN1CCCNCCNCCCNCC1]

Additional CXCR4 antagonists that may be used in conjunction with the compositions and methods described herein include variants of plenxafor, such as a compound described in U.S. Pat. No. 5,583,131, the disclosure of which is incorporated herein by reference as it pertains to CXCR4 antagonists. In some embodiments, the CXCR4 antagonist may be a compound selected from the group consisting of: 1,1′-[1,3-phenylenebis(methylene)]-bis-1,4,8,11-tetra-azacyclotetradecane; 1,1′-[1,4-phenylene-bis-(methylene)]-bis-1,4,8,11-tetraazacyclotetradecane; bis-zinc or bis-copper complex of 1,1′-[1,4-phenylene-bis-(methylene)]-bis-1,4,8,11-tetraazacyclotetradecane; 1,1′-[3,3′-biphenylene-bis-(methylene)]-bis-1,4,8,11-tetraazacyclotetradecane; 11,11′-[1,4-phenylene-bis-(methylene)]-bis-1,4,7,11-tetraazacyclotetradecane; 1,11′-[1,4-phenylene-bis-(methylene)]-1,4,8,11-tetraazacyclotetradecane-1,4,7,11-tetraazacyclotetradecane; 1,1′-[2,6-pyridine-bis-(methylene)]-bis-1,4,8,11-tetraazacyclotetradecane; 1,1-[3,5-pyridine-bis-(methylene)]-bis-1,4,8,11-tetraazacyclotetradecane; 1,1′-[2,5-thiophene-bis-(methylene)]-bis-1,4,8,11-tetraazacyclotetradecane; 1,1′-[4,4′-(2,2′-bipyridine)-bis-(methylene)]-bis-1,4,8,11-tetraazacyclotetradecane; 1,1′-[2,9-(1,10-phenanthroline)-bis-(methylene)]-bis-1,4,8,11-tetraazacyclotetradecane; 1,1′-[1,3-phenylene-bis-(methylene)]-bis-1,4,7,10-tetraazacyclotetradecane; 1,1′-[1,4-phenylene-bis-(methylene)]-bis-1,4,7,10-tetraazacyclotetradecane; 1′-[5-nitro-1,3-phenylenebis(methylene)]bis-1,4,8,11-tetraazacyclotetradecane; 1′,1′-[2,4,5,6-tetrachloro-1,3-phenyleneis(methylene)]bis-1,4,8,11-tetraazacyclotetradecane; 1,1′-[2,3,5,6-tetra-fluoro-1,4-phenylenebis(methylene)]bis-1,4,8,11-tetraazacyclotetradecane; 1,1′-[1,4-naphthylene-bis-(methylene)]bis-1,4,8,11-tetraazacyclotetradecane; 1,1′-[1,3-phenylenebis-(methylene)]bis-1,5,9-triazacyclododecane; 1,1′-[1,4-phenylene-bis-(methylene)]-1,5,9-triazacyclododecane; 1,1′-[2,5-dimethyl-1,4-phenylenebis-(methylene)]-bis-1,4,8,11-tetraazacyclotetradecane; 1,1′-[2,5-dichloro-1,4-phenylenebis-(methylene)]-bis-1,4,8,11-tetraazacyclotetradecane; 1,1′-[2-bromo-1,4-phenylenebis-(methylene)]-bis-1,4,8,11-tetraazacyclotetradecane; and 1,1′-[6-phenyl-2,4-pyridinebis-(methylene)]-bis-1,4,8,11-tetraazacyclotetradecane.

In some embodiments, the CXCR4 antagonist is a compound described in US 2006/0035829, the disclosure of which is incorporated herein by reference as it pertains to CXCR4 antagonists. In some embodiments, the CXCR4 antagonist may be a compound selected from the group consisting of 3,7,11,17-tetraazabicyclo(13.3.1)heptadeca-1(17),13,15-triene; 4,7,10,17-tetraazabicyclo(13.3.1)heptadeca-1(17),13,15-triene; 1,4,7,10-tetraazacyclotetradecane; 1,4,7-triazacyclotetradecane; and 4,7,10-triazabicyclo(13.3.1)heptadeca-1(17),13,15-triene.

The CXCR4 antagonist may be a compound described in WO 2001/044229, the disclosure of which is incorporated herein by reference as it pertains to CXCR4 antagonists. In some embodiments, the CXCR4 antagonist may be a compound selected from the group consisting of: N-[4-(11-fluoro-1,4,7-triazacyclotetradecanyl)-1,4-phenylenebis(methylene)]-2-(aminomethyl)pyridine; N-[4-(11,11-difluoro-1,4,7-triazacyclotetradecanyl)-1,4-phenylenebis(methylene)]-2-(aminomethyl)pyridine; N-[4-(1,4,7-triazacyclotetradecan-2-onyl)-1,4-phenylenebis(methylene)]-2-(aminomethyl)pyridine; N-[12-(5-oxa-1,9-diazacyclotetradecanyl)-1,4-phenylenebis(methylene)]-2-(aminomethyl)pyridine; N-[4-(11-oxa-1,4,7-triazacyclotetradecanyl)-1,4-phenylenebis(methylene)]-2-(aminomethyl)pyridine; N-[4-(11-thia-1,4,7-triazacyclotetradecanyl)-1,4-phenylenebis(methylene)]-2-(aminomethyl)pyridine; N-[4-(11-sulfoxo-1,4,7-triazacyclotetradecanyl)-1,4-phenylenebis(methylene)]-2-(aminomethyl)pyridine; N-[4-(11-sulfono-1,4,7-triazacyclotetradecanyl)-1,4-phenylenebis(methylene)]-2-(aminomethyl)pyridine; and N-[4-(3-carboxo-1,4,7-triazacyclotetradecanyl)-1,4-phenylenebis(methylene)]-2-(aminomethyl)pyridine.

Additional CXCR4 antagonists useful in conjunction with the compositions and methods described herein include compounds described in WO 2000/002870, the disclosure of which is incorporated herein by reference as it pertains to CXCR4 antagonists. In some embodiments, the CXCR4 antagonist may be a compound selected from the group consisting of: N-[1,4,8,11-tetraazacyclotetra-decanyl-1,4-phenylenebis-(methylene)]-2-(aminomethyl)pyridine; N-[1,4,8,11-tetraazacyclotetra-decanyl-1,4-phenylenebis(methylene)]-N-methyl-2-(aminomethyl)pyridine; N-[1,4,8,11-tetraazacyclotetra-decanyl-1,4-phenylenebis(methylene)]-4-(aminomethyl)pyridine; N-[1,4,8,11-tetraazacyclotetra-decanyl-1,4-phenylenebis(methylene)]-3-(aminomethyl)pyridine; N-[1,4,8,11-tetraazacyclotetra-decanyl-1,4-phenylenebis(methylene)]-(2-aminomethyl-5-methyl)pyrazine; N-[1,4,8,11-tetraazacyclotetra-decanyl-1,4-phenylenebis(methylene)]-2-(aminoethyl) pyridine; N-[1,4,8,11-tetraazacyclotetra-decanyl-1,4-phenylenebis(methylene)]-2-(aminomethyl)thiophene; N-[1,4,8,11-tetraazacyclotetra-decanyl-1,4-phenylenebis(methylene)]-2-(aminomethyl)mercaptan; N-[1,4,8,11-tetraazacyclotetra-decanyl-1,4-phenylenebis(methylene)]-2-amino benzylamine; N-[1,4,8,11-tetraazacyclotetra-decanyl-1,4-phenylenebis(methylene)]-4-amino benzylamine; N-[1,4,8,11-tetraazacyclotetra-decanyl-1,4-phenylenebis(methylene)]-4-(aminoethyl)imidazole; N-[1,4,8,11-tetraazacyclotetra-decanyl-1,4-phenylenebis(methylene)]-benzylamine; N-[4-(1,4,7-triazacyclotetra-decanyl)-1,4-phenylenebis(methylene)]-2-(aminomethyl)pyridine; N-[7-(4,7,10,17-tetraazabicyclo[13.3.1]heptadeca-1(17),13,15-trienyl)-1,4-phenylenebis(methylene)]-2-(aminomethyl)pyridine; N-[7-(4,7,10-triazabicyclo[13.3.1]heptadeca-1(17),13,15-trienyl)-1,4-phenylenebis(methylene)]-2-(aminomethyl)pyridine; N-[1-(1,4,7-triazacyclotetra-decanyl)-1,4-phenylenebis(methylene)]-2-(aminomethyl)pyridine; N-[4-[4,7,10,17-tetraazabicyclo[13.3.1]heptadeca-1(17),13,15-trienyl]-1,4-phenylenebis(methylene)]-2-(aminomethyl)pyridine; N-[4-[4,7,10-triazabicyclo[13.3.1]heptadeca-1(17),13,15-trienyl]-1,4-phenylenebis(methylene)]-2-(aminomethyl)pyridine; N-[1,4,8,11-tetraazacyclotetradecanyl-1,4-phenylenebis(methylene)]-purine; 1-[1,4,8,11-tetraazacyclotetradecanyl-1,4-phenylenebix(methylene)]-4-phenylpiperazine; N-[4-(1,7-diazacyclotetradecanyl)-1,4-phenylenebis(methylene)]-2-(aminomethyl)pyridine; and N-[7-(4,10-diazabicyclo[13.3.1]heptadeca-1(17),13,15-trienyl)-1,4-phenylenebis(methylene)]-2-(aminomethyl)pyridine.

In some embodiments, the CXCR4 antagonist is a compound selected from the group consisting of 1-[2,6-dimethoxypyrid-4-yl(methylene)]-1,4,8,11-tetraazacyclotetradecane; 1-[2-chloropyrid-4-yl(methylene)]-1,4,8,11-tetraazacyclotetradecane; 1-[2,6-dimethylpyrid-4-yl(methylene)]-1,4,8,11-tetraazacyclotetradecane; 1-[2-methylpyrid-4-yl(methylene)]-1,4,8,11-tetraazacyclotetradecane; 1-[2,6-dichloropyrid-4-yl(methylene)]-1,4,8,11-tetraazacyclotetradecane; 1-[2-chloropyrid-5-yl(methylene)]-1,4,8,11-tetraazacyclotetradecane; and 7-[4-methylphenyl (methylene)]-4,7,10,17-tetraazabicyclo[13.3.1]heptadeca-1(17).13,15-triene.

In some embodiments, the CXCR4 antagonist is a compound described in U.S. Pat. No. 5,698,546, the disclosure of which is incorporated herein by reference as it pertains to CXCR4 antagonists. In some embodiments, the CXCR4 antagonist may be a compound selected from the group consisting of 7,7′-[1,4-phenylene-bis(methylene)]bis-3,7,11,17-tetraazabicyclo[13.3.1]heptadeca-1(17),13,15-triene; 7,7′-[1,4-phenylene-bis(methylene)]bis[15-chloro-3,7,11,17-tetraazabicyclo [13.3.1]heptadeca-1 (17),13,15-triene]; 7,7′-[1,4-phenylene-bis(methylene)]bis[15-methoxy-3,7,11,17-tetraazabicyclo[13.3.1]heptadeca-1(17),13,15-triene]; 7,7′-[1,4-phenylene-bis(methylene)]bis-3,7,11,17-tetraazabicyclo[13.3.1]-heptadeca-13,16-triene-15-one; 7,7′-[1,4-phenylene-bis(methylene)]bis-4,7,10,17-tetraazabicyclo[13.3.1]-heptadeca-1(17),13,15-triene; 8,8′-[1,4-phenylene-bis(methylene)]bis-4,8,12,19-tetraazabicyclo[15.3.1]nonadeca-1(19),15,17-triene; 6,6′-[1,4-phenylene-bis(methylene)]bis-3,6,9,15-tetraazabicyclo[11.3.1]pentadeca-1 (15),11,13-triene; 6,6′-[1,3-phenylene-bis(methylene)]bis-3,6,9,15-tetraazabicyclo[11.3.1]pentadeca-1 (15),11,13-triene; and 17,17′-[1,4-phenylene-bis(methylene)]bis-3,6,14,17,23,24-hexaazatricyclo[17.3.1.1.^{8,12}]tetracosa-1(23),8,10,12(24),19,21-hexaene.

In some embodiments, the CXCR4 antagonist is a compound described in U.S. Pat. No. 5,021,409, the disclosure of which is incorporated herein by reference as it pertains to CXCR4 antagonists. In some embodiments, the CXCR4 antagonist may be a compound selected from the group consisting at 2,2′-bicyclam, 6,6′-bicyclam; 3,3′-(bis-1,5,9,13-tetraaza cyclohexadecane); 3,3′-(bis-1,5,8,11,14-pentaazacyclohexadecane); methylene (or polymethylene) di-1-N-1,4,8,11-tetraaza cyclotetradecane; 3,3′-bis-1,5,9,13-tetraazacyclohexadecane; 3,3′-bis-1,5,8,11,14-pentaazacyclohexadecane; 5,5′-bis-1,4,8,11-tetraazacyclotetradecane; 2,5′-bis-1,4,8,11-tetraazacyclotetradecane; 2,6′-bis-1,4,8,11-tetraazacyclotetradecane; 11,11′-(1,2-ethanediyl)bis-1,4,8,11-tetraazacyclotetradecane; 11,11′-(1,2-propanediyl)bis-1,4,8,11-tetraazacyclotetradecane; 11,11′-(1,2-butanediyl)bis-1,4,8,11-tetraazacyclotetradecane; 11,11′-(1,2-pentanediyl)bis-1,4,8,11-tetraazacyclotetradecane; and 11,11′-(1,2-hexanediyl)bis-1,4,8,11-tetraazacyclotetradecane.

In some embodiments, the CXCR4 antagonist is a compound described in WO 2000/056729, the disclosure of which is incorporated herein by reference as it pertains to CXCR4 antagonists. In some embodiments, the CXCR4 antagonist may be a compound selected from the group consisting of: N-(2-pyridinylmethyl)-N′-(6,7,8,9-tetrahydro-5H-cyclohepta[b]pyridin-9-yl)-1,4-benzenedimethanamine; N-(2-pyridinylmethyl)-N′-(5,6,7,8-tetrahydro-8-quinolinyl)-1,4-benzenedimethanamine; N-(2-pyridinylmethyl)-N′-(6,7-dihydro-5H-cyclopenta[b]pyridin-7-yl)-1,4-benzenedimethanamine; N-(2-pyridinylmethyl)-N′-(1,2,3,4-tetrahydro-1-naphthalenyl)-1,4-benzenedimethanamine; N-(2-pyridinylmethyl)-N′-(1-naphthalenyl)-1,4-benzenedimethanamine; N-(2-pyridinylmethyl)-N′-(8-quinolinyl)-1,4-benzenedimethanamine; N-(2-pyridinylmethyl)-N′-[2-[(2-pyridinylmethyl)amino]ethyl]-N′-(1-methyl-1,2,3,4-tetrahydro-8-quinolinyl)-1,4-benzenedimethanamine; N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzenedimethanamine; N-(2-pyridinylmethyl)-N′-(1,2,3,4-tetrahydro-8-quinolinyl)-1,4-benzenedimethanamine; N-(2-pyridinylmethyl)-N′-[2-[(1H-imidazol-2-ylmethyl)amino]ethyl]-N′-(1,2,3,4-tetrahydro-1-naphthalenyl)-1,4-benzenedimethanamine; N-(2-pyridinylmethyl)-N′-(2-phenyl-5,6,7,8-tetrahydro-8-quinolinyl)-1,4-benzenedimethanamine; N,N′-bis(2-pyridinylmethyl)-N′-(2-phenyl-5,6,7,8-tetrahydro-8-quinolinyl)-1,4-benzenedimethanamine; N-(2-pyridinylmethyl)-N′-(5,6,7,8-tetrahydro-5-quinolinyl)-1,4-benzenedimethanamine; N-(2-pyridinylmethyl)-N′-(1H-imidazol-2-ylmethyl)-N′-(5,6,7,8-tetrahydro-5-quinolinyl)-1,4-benzenedimethanamine; N-(2-pyridinylmethyl)-N′-(1H-imidazol-2-ylmethyl)-N′-(5,6,7,8-tetrahydro-8-quinolinyl)-1,4-benzenedimethanamine; N-(2-pyridinylmethyl)-N′-[(2-amino-3-phenyl)propyl]-N′-(5,6,7,8-tetrahydro-8-quinolinyl)-1,4-benzenedimethanamine; N-(2-pyridinylmethyl)-N′-(1H-imidazol-4-ylmethyl)-N′-(5,6,7,8-tetrahydro-8-quinolinyl)-1,4-benzenedimethanamine; N-(2-pyridinylmethyl)-N′-(2-quinolinylmethyl)-N′-(5,6,7,8-tetrahydro-8-quinolinyl)-1,4-benzenedimethanamine; N-(2-pyridinylmethyl)-N′-(2-(2-naphthoyl)aminoethyl)-N′-(5,6,7,8-tetrahydro-8-quinolinyl)-1,4-benzenedimethanamine; N-(2-pyridinylmethyl)-N′—[(S)-(2-acetylamino-3-phenyl)propyl]-N′-(5,6,7,8-tetrahydro-8-quinolinyl)-1,4-benzenedimethanamine; N-(2-pyridinylmethyl)-N′—[(S)-(2-acetylamino-3-phenyl)propyl]-N′-(5,6,7,8-tetrahydro-8-quinolinyl)-1,4-benzenedimethanamine; N-(2-pyridinylmethyl)-N′-[3-((2-naphthalenylmethyl)amino)propyl]-N′-(5,6,7,8-tetrahydro-8-quinolinyl)-1,4-benzenedimethanamine; N-(2-pyridinylmethyl)-N′-[2-(S)-pyrollidinylmethyl]-N′-(5,6,7,8-tetrahydro-8-quinolinyl)-1,4-benzenedimethanamine; N-(2-pyridinylmethyl)-N′-[2-(R)-pyrollidinylmethyl]-N′-(5,6,7,8-tetrahydro-8-quinolinyl)-1,4-benzenedimethanamine; N-(2-pyridinylmethyl)-N′-[3-pyrazolylmethyl]-N′-(5,8,7,8-tetrahydro-8-quinolinyl)-1,4-benzenedimethanamine; N-(2-pyridinylmethyl)-N′-[2-pyrrolylmethyl]-N′-(5,6,7,8-tetrahydro-8-quinolinyl)-1,4-benzenedimethanamine; N-(2-pyridinylmethyl)-N′-[2-thiopheneylmethyl]-N′-(5,6,7,8-tetrahydro-8-quinolinyl)-1,4-benzenedimethanamine; N-(2-pyridinylmethyl)-N′-[2-thiazolylmethyl]-N′-(5,6,7,8-tetrahydro-8-quinolinyl)-1,4-benzenedimethanamine; N-(2-pyridinylmethyl)-N′-[2-furanylmethyl]-N′-(5,6,7,8-tetrahydro-8-quinolinyl)-1,4-benzenedimethanamine; N-(2-pyridinylmethyl)-N′-[2-[(phenylmethyl)amino]ethyl]-N′-(5,6,7,8-tetrahydro-8-quinolinyl)-1,4-benzenedimethanamine; N-(2-pyridinylmethyl)-N′-(2-aminoethyl)-N′-(5,6,7,8-tetrahydro-8-quinolinyl)-1,4-benzenedimethanamine; N-(2-pyridinylmethyl)-N′-3-pyrrolidinyl-N′-(5,6,7,8-tetrahydro-8-quinolinyl)-1,4-benzenedimethanamine N-(2-pyridinylmethyl)-N′-4-piperidinyl-N′-(5,6,7,8-tetrahydro-8-quinolinyl)-1,4-benzenedimethanamine; N-(2-pyridinylmethyl)-N′-[2-[(phenyl)amino]ethyl]-N′-(5,6,7,8-tetrahydro-8-quinolinyl)-1,4-benzenedimethanamine; N-(2-pyridinylmethyl)-N′-(7-methoxy-1,2,3,4-tetrahydro-2-naphthalenyl)-1,4-benzenedimethanamine; N-(2-pyridinylmethyl)-N′-(6-methoxy-1,2,3,4-tetrahydro-2-naphthalenyl)-1,4-benzenedimethanamine; N-(2-pyridinylmethyl)-N′-(1-methyl-1,2,3,4-tetrahydro-2-naphthalenyl)-1,4-benzenedimethanamine; N-(2-pyridinylmethyl)-N′-(7-methoxy-3,4-dihydronaphthalenyl)-1-(aminomethyl)-4-benzamide; N-(2-pyridinylmethyl)-N′-(6-methoxy-3,4-dihydronaphthalenyl)-1-(aminomethyl)-4-benzamide; N-(2-pyridinylmethyl)-N′-(1H-imidazol-2-ylmethyl)-N′-(7-methoxy-1,2,3,4-tetrahydro-2-naphthalenyl)-1,4-benzenedimethanamine; N-(2-pyridinylmethyl)-N′-(8-hydroxy-1,2,3,4-tetrahydro-2-naphthalenyl)-1,4-benzenedimethanamine; N-(2-pyridinylmethyl)-N′-(1H-imidazol-2-ylmethyl)-N′-(8-hydroxy-1,2,3,4-tetrahydro-2-naphthalenyl)-1,4-benzenedimethanamine; N-(2-pyridinylmethyl)-N′-(8-Fluoro-1,2,3,4-tetrahydro-2-naphthalenyl)-1,4-benzenedimethanamine; N-(2-pyridinylmethyl)-N′-(1H-imidazol-2-ylmethyl)-N′-(8-Fluoro-1,2,3,4-tetrahydro-2-naphthalenyl)-1,4-benzenedimethanamine; N-(2-pyridinylmethyl)-N′-(5,6,7,8-tetrahydro-7-quinolinyl)-1,4-benzenedimethanamine; N-(2-pyridinylmethyl)-N′-(1H-imidazol-2-ylmethyl)-N′-(5,6,7,8-tetrahydro-7-quinolinyl)-1,4-benzenedimethanamine; N-(2-pyridinylmethyl)-N′-[2-[(2-naphthalenylmethyl)amino]ethyl]-N′-(5,6,7,8-tetrahydro-8-quinolinyl)-1,4-benzenedimethanamine; N-(2-pyridinylmethyl)-N′-[2-(isobutylamino)ethyl)]-N′-(5,6,7,8-tetrahydro-8-quinolinyl)-1,4-benzenedimethanamine; N-(2-pyridinylmethyl)-N′-[2-[(2-pyridinylmethyl)amino]ethyl]-N′-(5,6,7,8-tetrahydro-8-quinolinyl)-1,4-benzenedimethanamine; N-(2-pyridinylmethyl)-N′-[2-[(2-furanylmethyl)amino]ethyl]-N′-(5,6,7,8-tetrahydro-8-quinolinyl)-1,4-benzenedimethanamine; N-(2-pyridinylmethyl)-N′-(2-guanidinoethyl)-N′-(5,6,7,8-tetrahydro-8-quinolinyl)-1,4-benzenedimethanamine; N-(2-pyridinylmethyl)-N′-[2-[bis-[(2-methoxy)phenylmethyl]amino]ethyl]-N′-(5,6,7,8-tetrahydro-8-quinolinyl)-1,4-benzenedimethanamine; N-(2-pyridinylmethyl)-N′-[2-[(1H-imidazol-4-ylmethyl)amino]ethyl]-N′-(5,6,7,8-tetrahydro-8-quinolinyl)-1,4-benzenedimethanamine; N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine; N-(2-pyridinylmethyl)-N′-[2-(phenylureido)ethyl]-N′-(5,6,7,8-tetrahydro-8-quinolinyl)-1,4-benzenedimethanamine; N-(2-pyridinylmethyl)-N′—[[N″-(n-butyl)carboxamido]methyl]-N-(5,6,7,8-tetrahydro-8-quinolinyl)-1,4-benzenedimethanamine; N-(2-pyridinylmethyl)-N′-(carboxamidomethyl)-N′-(5,6,7,8-tetrahydro-8-quinolinyl)-1,4-benzenedimethanamine; N-(2-pyridinylmethyl)-N′—[(N″-phenyl)carboxamidomethyl]-N′-(5,6,7,8-tetrahydro-8-quinolinyl)-1,4-benzenedimethanamine; N-(2-pyridinylmethyl)-N′-(carboxymethyl)-N′-(5,6,7,8-tetrahydro-8-quinolinyl)-1,4-benzenedimethanamine; N-(2-pyridinylmethyl)-N′-(phenylmethyl)-N′-(5,6,7,8-tetrahydro-8-quinolinyl)-1,4-benzenedimethanamine; N-(2-pyridinylmethyl)-N′-(1H-benzimidazol-2-ylmethyl)-N′-(5,6,7,8-tetrahydro-8-quinolinyl)-1,4-benzenedimethanamine; N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine (hydrobromide salt); N-(2-pyridinylmethyl)-N′-(5-nitro-1H-benzimidazol-2-ylmethyl)-N′-(5,6,7,8-tetrahydro-8-quinolinyl)-1,4-benzenedimethanamine; N-(2-pyridinylmethyl)-N′-[(1H)-5-azabenzimidazol-2-ylmethyl]-N′-(5,6,7,8-tetrahydro-8-quinolinyl)-1,4-benzenedimethanamine; N-(2-pyridinylmethyl)-N-(4-phenyl-1H-imidazol-2-ylmethyl)-N′-(5,6,7,8-tetrahydro-8-quinolinyl)-1,4-benzenedimethanamine; N-(2-pyridinylmethyl)-N′-[2-(2-pyridinyl)ethyl]-N′-(5,6,7,8-tetrahydro-8-quinolinyl)-1,4-benzenedimethanamine; N-(2-pyridinylmethyl)-N′-(2-benzoxazolyl)-N′-(5,6,7,8-tetrahydro-8-quinolinyl)-1,4-benzenedimethanamine; N-(2-pyridinylmethyl)-N′-(trans-2-aminocyclohexyl)-N′-(5,6,7,8-tetrahydro-8-quinolinyl)-1,4-benzenedimethanamine; N-(2-pyridinylmethyl)-N′-(2-phenylethyl)-N′-(5,6,7,8-tetrahydro-8-quinolinyl)-1,4-benzenedimethanamine; N-(2-pyridinylmethyl)-N′-(3-phenylpropyl)-N′-(5,6,7,8-tetrahydro-8-quinolinyl)-1,4-benzenedimethanamine; N-(2-pyridinylmethyl)-N′-(trans-2-aminocyclopentyl)-N-(5,6,7,8-tetrahydro-8-quinolinyl)-1,4-benzenedimethanamine; N-[[4-[[(2-pyridinylmethyl)amino]methyl]phenyl]methyl]-N-(5,6,7,8-tetrahydro-8-quinolinyl)-glycinamide; N-[[4-[[(2-pyridinylmethyl)amino]methyl]phenyl]methyl]-N-(5,6,7,8-tetrahydro-8-quinolin-(L)-alaninamide; N-[[4-[[(2-pyridinylmethyl)amino]methyl]phenyl]methyl]-N-(5,6,7,8-tetrahydro-8-quinolinyl)-(L)-aspartamide; N-[[4-[[(2-pyridinylmethyl)amino]methyl]phenyl]methyl]-N-(5,6,7,8-tetrahydro-8-quinolinyl)-pyrazinamide; N-[[4-[[(2-pyridinylmethyl)amino]methyl]phenyl]methyl]-N-(5,6,7,8-tetrahydro-8-quinolinyl)-(L)-prolinamide; N-[[4-[[(2-pyridinylmethyl)amino]methyl]phenyl]methyl]-N-(5,6,7,8-tetrahydro-8-quinolinyl)-(L)-lysinamide; N-[[4-[[(2-pyridinylmethyl)amino]methyl]phenyl]methyl]-N-(5,6,7,8-tetrahydro-8-quinolinyl)-benzamide; N-[[4-[[(2-pyridinylmethyl)amino]methyl]phenyl]methyl]-N-(5,6,7,8-tetrahydro-8-quinolinyl)-picolinamide; N′-Benzyl-N-[[4-[[(2-pyridinylmethyl)amino]methyl]phenyl]methyl]-N-(5,6,7,8-tetrahydro-8-quinolinyl)-urea; N′-phenyl-N-[[4-[[(2-pyridinylmethyl)amino]methyl]phenyl]methyl]-N-(5,6,7,8-tetrahydro-8-quinolinyl)-urea; N-(6,7,8,9-tetrahydro-5H-cyclohepta[bacteriapyridin-9-yl)-4-[[(2-pyridinylmethyl)amino]methyl]benzamide; N-(5,6,7,8-tetrahydro-8-quinolinyl)-4-[[(2-pyridinylmethyl)amino]methyl]benzamide; N,N′-bis(2-pyridinylmethyl)-N′-(5,6,7,8-tetrahydro-8-quinolinyl)-1,4-benzenedimethanamine; N,N′-bis(2-pyridinylmethyl)-N′-(6,7,8,9-tetrahydro-5H-cyclohepta[bacteriapyridin-9-yl)-1,4-benzenedimethanamine; N,N′-bis(2-pyridinylmethyl)-N′-(6,7-dihydro-5H-cyclopenta[bacteriapyridin-7-yl)-1,4-benzenedimethanamine; N,N′-bis(2-pyridinylmethyl)-N′-(1,2,3,4-tetrahydro-1-naphthalenyl)-1,4-benzenedimethanamine; N,N′-bis(2-pyridinylmethyl)-N′-[(5,6,7,8-tetrahydro-8-quinolinyl)methyl]-1,4-benzenedimethanamine; N,N′-bis(2-pyridinylmethyl)-N′[(6,7-dihydro-5H-cyclopenta]bacteriapyridin-7-yl)methyl-1,4-benzenedimethanamine; N-(2-pyridinylmethyl)-N-(2-methoxyethyl)-N′-(5,6,7,8-tetrahydro-8-quinolinyl)-1,4-benzenedimethanamine; N-(2-pyridinylmethyl)-N-[2-(4-methoxyphenyl)ethyl]-N′-(5,6,7,8-tetrahydro-8-quinolinyl)-1,4-benzenedimethanamine; N,N′-bis(2-pyridinylmethyl)-1,4-(5,6,7,8-tetrahydro-8-quinolinyl)benzenedimethanamine; N-[(2,3-dimethoxyphenyl)methyl]-N′-(2-pyridinylmethyl)-N-(5,6,7,8-tetrahydro-8-quinolinyl)-1,4-benzenedimethanamine; N,N′-bis(2-pyridinylmethyl)-N-[1-(N″-phenyl-N″-methylureido)-4-piperidinyl]-1,3-benzenedimethanamine; N,N′-bis(2-pyridinylmethyl)-N—[N″-p-toluenesulfonylphenylalanyl)-4-piperidinyl]-1,3-benzenedimethanamine; N,N′-bis(2-pyridinylmethyl)-N-[1-[3-(2-chlorophenyl)-5-methyl-isoxazol-4-oyl]-4-piperidinyl]-1,3-benzenedimethanamine; N-[(2-hydroxyphenyl)methyl]-N′-(2-pyridinylmethyl)-N-(6,7,8,9-tetrahydro-5H-cyclohepta]bacteriapyridin-9-yl)-1,4-benzenedimethanamine; N-[(4-cyanophenyl)methyl]-N′-(2-pyridinylmethyl)-N-(6,7,8,9-tetrahydro-5H-cyclohepta]bacteriapyridin-9-yl)-1,4-benzenedimethanamine; N-[(4-cyanophenyl)methyl]-N′-(2-pyridinylmethyl)-N-(5,6,7,8-tetrahydro-8-quinolinyl)-1,4-benzenedimethanamine; N-[(4-acetamidophenyl)methyl]-N′-(2-pyridinylmethyl)-N-(5,6,7,8-tetrahydro-8-quinolinyl)-1,4-benzenedimethanamine; N-[(4-phenoxyphenyl)methyl]-N′-(2-pyridinylmethyl)-N-(6,7,8,9-tetrahydro-5H-cyclohepta]bacteriapyridin-9-yl)-1,4-benzenedimethanamine; N-[(1-methyl-2-carboxamido)ethyl-N,N′-bis(2-pyridinylmethyl)-1,3-benzenedimethanamine; N-[(4-benzyloxyphenyl)methyl]-N′-(2-pyridinylmethyl)-N-(6,7,8,9-tetrahydro-5H-cyclohepta]bacteriapyridin-9-yl)-1,4-benzenedimethanamine; N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine; N-[1-(benzyl)-3-pyrrolidinyl]-N,N′-bis(2-pyridinylmethyl)-1,3-benzenedimethanamine; N-[[1-methyl-3-(pyrazol-3-yl)]propyl]-N,N′-bis(2-pyridinylmethyl)-1,3-benzenedimethanamine; N-[1-(phenyl)ethyl]-N,N′-bis(2-pyridinylmethyl)-1,3-benzenedimethanamine; N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine; N-[1-benzyl-3-carboxymethyl-4-piperidinyl]-N,N′-bis(2-pyridinylmethyl)-1,3-benzenedimethanamine; N-[(3,4-methylenedioxyphenyl)methyl]-N′-(2-pyridinylmethyl)-N-(5,6,7,8-tetrahydro-8-quinolinyl-1,4-benzenedimethanamine; N-(3-pyridinylmethyl)-N′-(2-pyridinylmethyl)-N-(6,7,8,9-tetrahydro-5H-cyclohepta[b]pyridin-9-yl)-1,4-benzenedimethanamine; N-[[1-methyl-2-(2-tolyl)carboxamido]ethyl]-N,N′-bis(2-pyridinylmethyl)-1,3-benzenedimethanamine; N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine; N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine; N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine; N—[H-imidazol-4-ylmethyl]-N,N′-bis(2-pyridinylmethyl)-1,3-benzenedimethanamine; N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine; N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine; N-[(3-cyanophenyl)methyl]-N′-(2-pyridinylmethyl)-N-(5,6,7,8-tetrahydro-8-quinolinyl)-1,4-benzenedimethanamine; N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine; N-(5-ethylthiophene-2-ylmethyl)-N′-(2-pyridinylmethyl)-N-(5,6,7,8-tetrahydro-8-quinolinyl)-1,4-benzenedimethanamine; N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine; N-[(2,6-difluorophenyl)methyl]-N′-(2-pyridinylmethyl)-N-(5,6,7,8-tetrahydro-8-quinolinyl)-1,4-benzenedimethanamine; N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine; N-(2-difluoromethoxyphenylmethyl)-N′-(2-pyridinylmethyl)-N-(5,6,7,8-tetrahydro-8-quinolinyl)-1,4-benzenedimethanamine; N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine; N,N′-bis(2-pyridinylmethyl)-N-[1-(N″-phenyl-N″-methylureido)-4-piperidinyl]-1,4-benzenedimethanamine; N,N′-bis(2-pyridinylmethyl)-N—[N″-p-toluenesulfonylphenylalanyl)-4-piperidinyl]-1,4-benzenedimethanamine; N-[1-(3-pyridinecarboxamido)-4-piperidinyl]-N,N′-bis(2-pyridinylmethyl)-1,4-benzenedimethanamine; N-[1-(cyclopropylcarboxamido)-4-piperidinyl]-N,N′-bis(2-pyridinylmethyl)-1,4-benzenedimethanamine; N-[1-(1-phenylcyclopropylcarboxamido)-4-piperidinyl]-N,N′-bis(2-pyridinylmethyl)-1,4-benzenedimethanamine; N-(1,4-benzodioxan-6-ylmethyl)-N′-(2-pyridinylmethyl)-N-(5,6,7,8-tetrahydro-8-quinolinyl)-1,4-benzenedimethanamine; N-[1-[3-(2-chlorophenyl)-5-methyl-isoxazol-4-carboxamido]-4-piperidinyl]-N,N′-bis(2-pyridinylmethyl)-1,4-benzenedimethanamine; N-[1-(2-thiomethylpyridine-3-carboxamido)-4-piperidinyl]-N,N′-bis(2-pyridinylmethyl)-1,4-benzenedimethanamine; N-[(2,4-difluorophenyl)methyl]-N′-(2-pyridinylmethyl)-N-(5,6,7,8-tetrahydro-8-quinolinyl)-1,4-benzenedimethanamine; N-(1-methylpyrrol-2-ylmethyl)-N′-(2-pyridinylmethyl)-N-(5,6,7,8-tetrahydro-8-quinolinyl)-1,4-benzenedimethanamine; N-[(2-hydroxyphenyl)methyl]-N′-(2-pyridinylmethyl)-N-(5,6,7,8-tetrahydro-8-quinolinyl)-1,4-benzenedimethanamine; N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine; N-(3-pyridinylmethyl)-N′-(2-pyridinylmethyl)-N-(5,6,7,8-tetrahydro-8-quinolinyl)-1,4-benzenedimethanamine; N-[2-(N″-morpholinomethyl)-1-cyclopentyl]-N,N′-bis(2-pyridinylmethyl)-1,4-benzenedimethanamine; N-[(1-methyl-3-piperidinyl)propyl]-N,N′-bis(2-pyridinylmethyl)-1,4-benzenedimethanamine; N-(1-methylbenzimidazol-2-ylmethyl)-N′-(2-pyridinylmethyl)-N-(5,6,7,8-tetrahydro-8-quinolinyl)-1,4-benzenedimethanamine; N-[1-(benzyl)-3-pyrrol idinyl]-N,N′-bis(2-pyridinylmethyl)-1,4-benzenedimethanamine; N-[[(1-phenyl-3-(N″-morpholino)]propyl]-N,N′-bis(2-pyridinylmethyl)-1,4-benzenedimethanamine; N-[1-(iso-propyl)-4-piperidinyl]-N,N′-bis(2-pyridinylmethyl)-1,4-benzenedimethanamine; N-[1-(ethoxycarbonyl)-4-piperidinyl]-N′-(2-pyridinylmethyl)-N-(5,6,7,8-tetrahydro-8-quinolinyl)-1,4-benzenedimethanamine; N-[(1-methyl-3-pyrazolyl)propyl]-N′-(2-pyridinylmethyl)-N-(5,6,7,8-tetrahydro-8-quinolinyl)-1,4-benzenedimethanamine; N-[1-methyl-2-(N″,N″-diethylcarboxamido)ethyl]-N,N′-bis(2-pyridinylmethyl)-1,4-benzenedimethanamine; N-[(1-methyl-2-phenylsulfonyl)ethyl]-N′-(2-pyridinylmethyl)-N-(5,6,7,8-tetrahydro-8-quinolinyl)-1,4-benzenedimethanamine; N-[(2-chloro-4,5-methylenedioxyphenyl)methyl]-N′-(2-pyridinylmethyl)-N-(5,6,7,8-tetrahydro-8-quinolinyl)-1,4-benzenedimethanamine; N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine; N-(1-acetoxyindol-3-ylmethyl)-N′-(2-pyridinylmethyl)-N-(6,7,8,9-tetrahydro-5H-cyclohepta[b]pyridin-9-yl)-1,4-benzenedimethanamine; N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine; N-(3-quinolylmethyl)-N′-(2-pyridinylmethyl)-N-(5,6,7,8-tetrahydro-8-quinolinyl)-1,4-benzenedimethanamine; N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine; N-(2-quinolylmethyl)-N′-(2-pyridinylmethyl)-N-(6,7,8,9-tetrahydro-5H-cyclohepta[b]pyridin-9-yl)-1,4-benzenedimethanamine; N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine; N-[1H-imidazol-2-ylmethyl]-N,N′-bis(2-pyridinylmethyl)-1,4-benzenedimethanamine; N-(3-quinolylmethyl)-N′-(2-pyridinylmethyl)-N-(6,7,8,9-tetrahydro-5H-cyclohepta[b]pyridin-9-yl)-1,4-benzenedimethanamine; N-(2-thiazolylmethyl)-N′-(2-pyridinylmethyl)-N-(6,7,8,9-tetrahydro-5H-cyclohepta[b]pyridin-9-yl)-1,4-benzenedimethanamine; N-(4-pyridinylmethyl)-N′-(2-pyridinylmethyl)-N-(6,7,8,9-tetrahydro-5H-cyclohepta[b]pyridin-9-yl)-1,4-benzenedimethanamine; N-[(5-benzyloxy)benzo[b]pyrrol-3-ylmethyl]-N,N′-bis(2-pyridinylmethyl)-1,4-benzenedimethanamine; N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine; N-[(4-methyl)-1H-imidazol-5-ylmethyl]-N,N′-bis(2-pyridinylmethyl)-1,4-benzenedimethanamine; N-[[(4-dimethylamino)-1-napthalenyl]methyl]-N,N′-bis(2-pyridinylmethyl)-1,4-benzenedimethanamine; N-[1,5-dimethyl-2-phenyl-3-pyrazolinone-4-ylmethyl]-N,N′-bis(2-pyridinylmethyl)-1,4-benzenedimethanamine; N-[1-[(1-acetyl-2-(R)-prolinyl]-4-piperidinyl]-N-[2-(2-pyridinyl)ethyl]-N′-(2-pyridinylmethyl)-1,3-benzenedimethanamine; N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine; N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine; N—[(N″-acetyltryptophanyl)-4-piperidinyl]-N-[2-(2-pyridinyl)ethyl]-N′-(2-pyridinylmethyl)-1,3-benzenedimethanamine; N—[(N″-benzoylvalinyl)-4-piperidinyl]-N-[2-(2-pyridinyl)ethyl]-N′-(2-pyridinylmethyl)-1,3-benzenedimethanamine; N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine; N-(4-pyridinylmethyl)-N′-(2-pyridinylmethyl)-N-(5,6,7,8-tetrahydro-8-quinolinyl)-1,4-benzenedimethanamine; N-(1-methylbenzimadazol-2-ylmethyl)-N′-(2-pyridinylmethyl)-N-(6,7,8,9-tetrahydro-5H-cyclohepta[b]pyridin-9-yl)-1,4-benzenedimethanamine; N-[1-butyl-4-piperidinyl]-N-[2-(2-pyridinyl)ethyl]-N′-(2-pyridinylmethyl)-1,3-benzenedimethanamine; N-[1-benzoyl-4-piperidinyl]-N-[2-(2-pyridinyl)ethyl]-N′-(2-pyridinylmethyl)-1,3-benzenedimethanamine; N-[1-(benzyl)-3-pyrrolidinyl]-N-[2-(2-pyridinyl)ethyl]-N′-(2-pyridinylmethyl)-1,3-benzenedimethanamine; N-[(1-methyl)benzo[b]pyrrol-3-ylmethyl]-N-[2-(2-pyridinyl)ethyl]-N′-(2-pyridinylmethyl)-1,3-benzenedimethanamine; N-[1H-imidazol-4-ylmethyl]-N-[2-(2-pyridinyl)ethyl]-N′-(2-pyridinylmethyl)-1,3-benzenedimethanamine; N-[1-(benzyl)-4-piperidinyl]-N-[2-(2-pyridinyl)ethyl]-N′-(2-pyridinylmethyl)-1,4-benzenedimethanamine; N-[1-methylbenzimidazol-2-ylmethyl]-N-[2-(2-pyridinyl)ethyl]-N′-(2-pyridinylmethyl)-1,4-benzenedimethanamine; N-[(2-phenyl)benzo[b]pyrrol-3-ylmethyl]-N-[2-(2-pyridinyl)ethyl]-N′-(2-pyridinylmethyl)-1,4-benzenedimethanamine; N-[(6-methylpyridin-2-yl)methyl]-N′-(2-pyridinylmethyl)-N-(5,6,7,8-tetrahydro-8-quinolinyl)-1,4-benzenedimethanamine; N-(3-methyl-1H-pyrazol-5-ylmethyl)-N′-(2-pyridinylmethyl)-N-(5,6,7,8-tetrahydro-8-quinolinyl)-1,3-benzenedimethanamine; N-[(2-methoxyphenyl)methyl]-N′-(2-pyridinylmethyl)-N-(5,6,7,8-tetrahydro-8-quinolinyl)-1,3-benzenedimethanamine; N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine; N-(benzyloxyethyl)-N′-(2-pyridinylmethyl)-N-(5,6,7,8-tetrahydro-8-quinolinyl)-1,3-benzenedimethanamine; N-[(2-ethoxy-1-naphthalenyl)methyl]-N′-(2-pyridinylmethyl)-N-(5,6,7,8-tetrahydro-8-quinolinyl)-1,3-benzenedimethanamine; N-[(6-methylpyridin-2-yl)methyl]-N′-(2-pyridinylmethyl)-N-(5,6,7,8-tetrahydro-8-quinolinyl)-1,3-benzenedimethanamine; 1-[[4-[[(2-pyridinylmethyl)amino]methyl]phenyl]methyl]guanidine; N-(2-pyridinylmethyl)-N-(8-methyl-8-azabicyclo[3.2.1]octan-3-yl)-1,4-benzenedimethanamine; 1-[[4-[[(2-pyridinylmethyl)amino]methyl]phenyl]methyl]homopiperazine; 1-[[3-[[(2-pyridinylmethyl)amino]methyl]phenyl]methyl]homopiperazine; trans and cis-1-[[4-[[(2-pyridinylmethyl)amino]methyl]phenyl]methyl]-3,5-piperidinediamine; N,N′-[1,4-Phenylenebis(methylene)]bis-4-(2-pyrimidyl)piperazine; 1-[[4-[[(2-pyridinylmethyl)amino]methyl]phenyl]methyl]-1-(2-pyridinyl)methylamine; 2-(2-pyridinyl)-5-[[(2-pyridinylmethyl)amino]methyl]-1,2,3,4-tetrahydroisoquinoline; 1-[[4-[[(2-pyridinylmethyl)amino]methyl]phenyl]methyl]-3,4-diaminopyrrolidine; 1-[[4-[[(2-pyridinylmethyl)amino]methyl]phenyl]methyl]-3,4-diacetylaminopyrrolidine; 8-[[4-[[(2-pyridinylmethyl)amino]methyl]phenyl]methyl]-2,5,8-triaza-3-oxabicyclo [4.3.0]nonane; and 8-[[4-[[(2-pyridinylmethyl)amino]methyl]phenyl]methyl]-2,5,8-triazabicyclo[4.3.0]nonane.

Additional CXCR4 antagonists that may be used to in conjunction with the compositions and methods described herein include those described in WO 2001/085196, WO 1999/050461, WO 2001/094420, and WO 2003/090512, the disclosures of each of which are incorporated herein by reference as they pertain to compounds that inhibit CXCR4 activity or expression.

In some embodiments, the CXCR4 antagonist is a peptide. For example, in some embodiments, the CXCR4 antagonist is BL-8040, having an IUPAC name of (3S,6S,9S,12R,17R,20S,23S,26S,29S,34aS)—N—((S)-1-amino-5-guanidino-1-oxopentan-2-yl)-26,29-bis(4-aminobutyl)-17-((S)-2-((S)-2-((S)-2-(4-fluorobenzamido)-5-guanidinopentanamido)-5-guanidinopentanamido)-3-(naphthalen-2-yl)propanamido)-6-(3-guanidinopropyl)-3,20-bis(4-hydroxybenzyl)-1,4,7,10,18,21,24,27,30-nonaoxo-9,23-bis(3-ureidopropyl)triacontahydro-1H,16H-pyrrolo[2,1-p][1,2]dithia[5,8,11,14,17,20,23,26,29]nonaazacyclodotriacontine-12-carboxamide, and having the structure shown below:

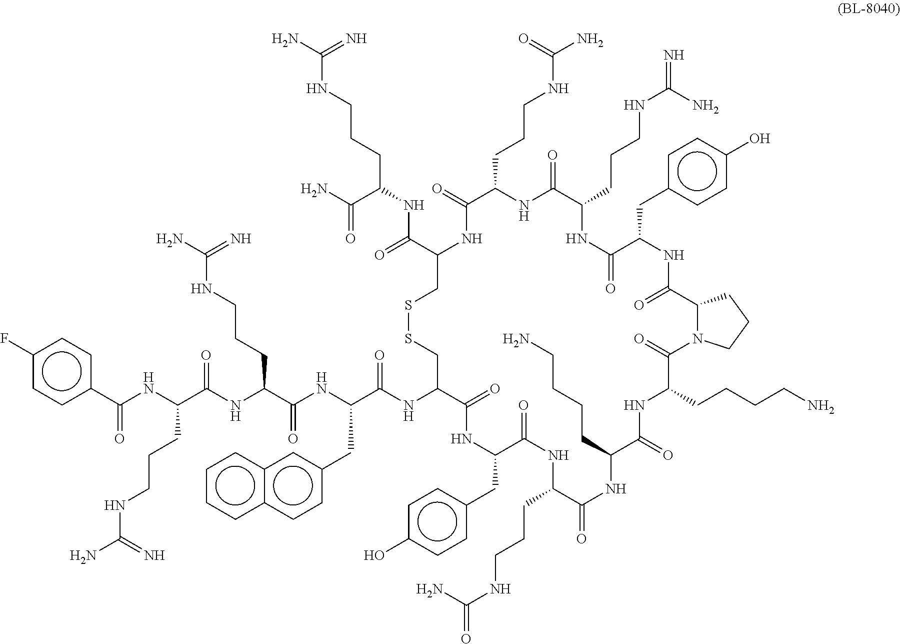

### CXCR2 Agonists

### Gro-β, Gro-β T, and Variants Thereof

Exemplary CXCR2 agonists that may be used in conjunction with the compositions and methods described herein are Gro-O and variants thereof. Gro-β (also referred to as growth-regulated protein β, chemokine (C—X—C motif) ligand 2 (CXCL2), and macrophage inflammatory protein 2-α (MIP2-α)) is a cytokine capable of mobilizing hematopoietic stem and progenitor cells, for example, by stimulating the release of proteases, and particularly MMP9, from peripheral neutrophils. Without being limited by mechanism, MMP9 may induce mobilization of hematopoietic stem and progenitor cells from stem cell niches, such as the bone marrow, to circulating peripheral blood by stimulating the degradation of proteins such as stem cell factor, its corresponding receptor, CD117, and CXCL12, all of which generally maintain hematopoietic stem and progenitor cells immobilized in bone marrow.

In addition to Gro-β, exemplary CXCR2 agonists that may be used in conjunction with the compositions and methods described herein are truncated forms of Gro-β, such as those that feature a deletion at the N-terminus of Gro-β of from 1 to 8 amino acids (e.g., peptides that feature an N-terminal deletion of 1 amino acids, 2 amino acids, 3 amino acids, 4 amino acids, 5 amino acids, 6 amino acids, 7 amino acids, or 8 amino acids). In some embodiments, CXCR2 agonists that may be used in conjunction with the compositions and methods described herein include Gro-β T, which is characterized by a deletion of the first four amino acids from the N-terminus of Gro-β. Gro-β and Gro-β T are described, for example, in U.S. Pat. No. 6,080,398, the disclosure of which is incorporated herein by reference in its entirety.

In addition, exemplary CXCR2 agonists that may be used in conjunction with the compositions and methods described herein are variants of Gro-β containing an aspartic acid residue in place of the asparagine residue at position 69 of SEQ ID NO: 1. This peptide is referred to herein as Gro-β N69D. Similarly, CXCR2 agonists that may be used with the compositions and methods described herein include variants of Gro-β T containing an aspartic acid residue in place of the asparagine residue at position 65 of SEQ ID NO: 2. This peptide is referred to herein as Gro-β T N65D T. Gro-β N69D and Gro-β T N65D are described, for example, in U.S. Pat. No. 6,447,766.

The amino acid sequences of Gro-β, Gro-β T, Gro-β N69D, and Gro-β T N65D are set forth in Table B, below.

TABLE B
Amino acid sequences of Gro-β and select
variants thereof

| SEQ | | |
|---|---|---|
| ID | | |
| NO. | Description | Amino Acid Sequence |
| 1 | Gro-β | APLATELRCQCLQTLQGIHLKNIQSVK |
| | | VKSPGPHCAQTEVIATLKNGQKACLN |
| | | PASPMVKKIIEKMLKNGKSN |
| 2 | Gro-β-T | TELRCQCLQTLQGIHLKNIQSVKVKS |
| | | PGPHCAQTEVIATLKNGQKACLNPAS |
| | | PMVKKIIEKMLKNGKSN |
| 3 | Gro-β N69D | APLATELRCQCLQTLQGIHLKNIQAVK |
| | | VKSPGPHCAQTEVIATLKNGQKACLN |
| | | PASPMVKKIIEKMLKDGKSN |
| 4 | Gro-β-T N65D | TELRCQCLQTLQGIHLKNIQSVKVKS |
| | | PGPHCAQTEVIATLKNGQKACLNPAS |
| | | PMVKKIIEKMLKDGKSN |

Additional CXCR2 agonists that may be used in conjunction with the compositions and methods described herein include other variants of Gro-β, such as peptides that have one or more amino acid substitutions, insertions, and/or deletions relative to Gro-β. In some embodiments, CXCR2 agonists that may be used in conjunction with the compositions and methods described herein include peptides having at least 85% sequence identity to the amino acid sequence of SEQ ID NO: 1 (e.g., a peptide having at least 85%, 90%, 95%, 96%, 97%, 98%, 99%, 99.5%, or 100% sequence identity to the amino acid sequence of SEQ ID NO: 1). In some embodiments, the amino acid sequence of the CXCR2 agonist differs from that of SEQ ID NO: 1 only by way of one or more conservative amino acid substitutions. In some embodiments, in some embodiments, the amino acid sequence of the CXCR2 agonist differs from that of SEQ ID NO: 1 by no more than 20, no more than 15, no more than 10, no more than 5, or no more than 1 nonconservative amino acid substitutions.

Additional examples of CXCR2 agonists useful in conjunction with the compositions and methods described herein are variants of Gro-3 T, such as peptides that have one or more amino acid substitutions, insertions, and/or deletions relative to Gro-β T. In some embodiments, the CXCR2 agonist may be a peptide having at least 85% sequence identity to the amino acid sequence of SEQ ID NO: 2 (e.g., a peptide having at least 85%, 90%, 95%, 96%, 97%, 98%, 99%, 99.5%, or 100% sequence identity to the amino acid sequence of SEQ ID NO: 2). In some embodiments, the amino acid sequence of the CXCR2 agonist differs from that of SEQ ID NO: 2 only by way of one or more conservative amino acid substitutions. In some embodiments, in some embodiments, the amino acid sequence of the CXCR2 agonist differs from that of SEQ ID NO: 2 by no more than 20, no more than 15, no more than 10, no more than 5, or no more than 1 nonconservative amino acid substitutions.

Additional examples of CXCR2 agonists useful in conjunction with the compositions and methods described herein are variants of Gro-β N69D, such as peptides that have one or more amino acid substitutions, insertions, and/or deletions relative to Gro-β N89D. In some embodiments, the CXCR2 agonist may be a peptide having at least 85% sequence identity to the amino acid sequence of SEQ ID NO: 3 (e.g., a peptide having at least 85%, 90%, 95%, 96%, 97%, 98%, 99%, 99.5%, or 100% sequence identity to the amino acid sequence of SEQ ID NO: 3). In some embodiments, the amino acid sequence of the CXCR2 agonist differs from that of SEQ ID NO: 3 only by way of one or more conservative amino acid substitutions. In some embodiments, in some embodiments, the amino acid sequence of the CXCR2 agonist differs from that of SEQ ID NO: 3 by no more than 20, no more than 15, no more than 10, no more than 5, or no more than 1 nonconservative amino acid substitutions.

Additional examples of CXCR2 agonists useful in conjunction with the compositions and methods described herein are variants of Gro-β T N65D, such as peptides that have one or more amino acid substitutions, insertions, and/or deletions relative to Gro-β T N65D. In some embodiments, the CXCR2 agonist may be a peptide having at least 85% sequence identity to the amino acid sequence of SEQ ID NO: 4 (e.g., a peptide having at least 85%, 90%, 95%, 96%, 97%, 98%, 99%, 99.5%, or 100% sequence identity to the amino acid sequence of SEQ ID NO: 4). In some embodiments, the amino acid sequence of the CXCR2 agonist differs from that of SEQ ID NO: 4 only by way of one or more conservative amino acid substitutions. In some embodiments, in some embodiments, the amino acid sequence of the CXCR2 agonist differs from that of SEQ ID NO: 4 by no more than 20, no more than 15, no more than 10, no more than 5, or no more than 1 nonconservative amino acid substitutions.

### Agonistic Anti-CXCR2 Antibodies and Antigen-Binding Fragments Thereof.

In some embodiments, the CXCR2 agonist is an antibody or antigen-binding fragment thereof that binds CXCR2 and activates CXCR2 signal transduction. In some embodiments, the CXCR2 agonist may be an antibody or antigen-binding fragment thereof that binds the same epitope on CXCR2 as Gro-β or a variant or truncation thereof, such as Gro-β T, as assessed, for example, by way of a competitive CXCR2 binding assay. In some embodiments, the CXCR2 agonist is an antibody or an antigen-binding fragment thereof that competes with Gro-O or a variant or truncation thereof, such as Gro-β T, for binding to CXCR2.

In some embodiments of any of the above aspects, the antibody or antigen-binding fragment thereof is selected from the group consisting of a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab)₂ molecule, and a tandem di-scFv. In some embodiments, the antibody has an isotype selected from the group consisting of IgG, IgA, IgM, IgD, and IgE.

### Synthetic CXCR2 Agonists

The peptidic CXCR2 agonists described herein, such as Gro-β, Gro-β T, and variants thereof, may be prepared synthetically, for instance, using solid phase peptide synthesis techniques. Systems and processes for performing solid phase peptide synthesis include those that are known in the art and have been described, for instance, in U.S. Pat. Nos. 9,169,287; 9,388,212; 9,206,222; 6,028,172; and 5,233,044, among others, the disclosures of each of which are incorporated herein by reference as they pertain to protocols and techniques for the synthesis of peptides on solid support. Solid phase peptide synthesis is a process in which amino acid residues are added to peptides that have been immobilized on a solid support, such as a polymeric resin (e.g., a hydrophilic resin, such as a polyethylene-glycol-containing resin, or hydrophobic resin, such as a polystyrene-based resin).

Peptides, such as those containing protecting groups at amino, hydroxy, thiol, and carboxy substituents, among others, may be bound to a solid support such that the peptide is effectively immobilized on the solid support. For example, the peptides may be bound to the solid support via their C termini, thereby immobilizing the peptides for subsequent reaction in at a resin-liquid interface.

The process of adding amino acid residues to immobilized peptides can include exposing a deprotection reagent to the immobilized peptides to remove at least a portion of the protection groups from at least a portion of the immobilized peptides. The deprotection reagent exposure step can be configured, for instance, such that side-chain protection groups are preserved, while N-terminal protection groups are removed. For instance, an exemplary amino protecting contains a fluorenylmethyloxycarbonyl (Fmoc) substituent. A deprotection reagent containing a strongly basic substance, such as piperidine (e.g., a piperidine solution in an appropriate organic solvent, such as dimethyl formamide (DMF)) may be exposed to the immobilized peptides such that the Fmoc protecting groups are removed from at least a portion of the immobilized peptides. Other protecting groups suitable for the protection of amino substituents include, for instance, the tert-butyloxycarbonyl (Boc) moiety. A deprotection reagent comprising a strong acid, such as trifluoroacetic acid (TFA) may be exposed to immobilized peptides containing a Boc-protected amino substituent so as to remove the Boc protecting group by an ionization process. In this way, peptides can be protected and deprotected at specific sites, such as at one or more side-chains or at the N- or C-terminus of an immobilized peptide so as to append chemical functionality regioselectively at one or more of these positions. This can be used, for instance, to derivatize a side-chain of an immobilized peptide, or to synthesize a peptide, e.g., from the C-terminus to the N-terminus.

The process of adding amino acid residues to immobilized peptides can include, for instance, exposing protected, activated amino acids to the immobilized peptides such that at least a portion of the activated amino acids are bonded to the immobilized peptides to form newly-bonded amino acid residues. For example, the peptides may be exposed to activated amino acids that react with the deprotected N-termini of the peptides so as to elongate the peptide chain by one amino acid. Amino acids can be activated for reaction with the deprotected peptides by reaction of the amino acid with an agent that enhances the electrophilicity of the backbone carbonyl carbon of the amino acid. For example, phosphonium and uronium salts can, in the presence of a tertiary base (e.g., diisopropylethylamine (DIPEA) and triethylamine (TEA), among others), convert protected amino acids into activated species (for example, BOP, PyBOP, HBTU, and TBTU all generate HOBt esters). Other reagents can be used to help prevent racemization that may be induced in the presence of a base. These reagents include carbodiimides (for example, DCC or WSCDI) with an added auxiliary nucleophile (for example, 1-hydroxy-benzotriazole (HOBt), 1-hydroxy-azabenzotriazole (HOAt), or HOSu) or derivatives thereof. Another reagent that can be utilized to prevent racemization is TBTU. The mixed anhydride method, using isobutyl chloroformate, with or without an added auxiliary nucleophile, can also be used, as well as the azide method, due to the low racemization associated with this reagent. These types of compounds can also increase the rate of carbodiimide-mediated couplings, as well as prevent dehydration of Asn and Gln residues. Typical additional reagents include also bases such as N,N-diisopropylethylamine (DIPEA), triethylamine (TEA) or N-methylmorpholine (NMM). These reagents are described in detail, for instance, in U.S. Pat. No. 8,546,350, the disclosure of which is incorporated herein in its entirety.

During the recombinant expression and folding of Gro-β and Gro-3 T in aqueous solution, a particular C-terminal asparagine residue (Asn69 within Gro-β and Asn65 within Gro-β T) is prone to deamidation. This process effectuates the conversion of the asparagine residue to aspartic acid. Without wishing to be bound by any theory, the chemical synthesis of Gro-β and Gro-O T may overcome this problem, for instance, by providing conditions that reduce the exposure of this asparagine residue to nucleophilic solvent. When prepared synthetically (i.e., chemically synthesized), for instance, using, e.g., the solid phase peptide synthesis techniques described above, synthetic Gro-s, Gro-β T, and variants thereof that may be used in conjunction with the compositions and methods described herein may have a purity of, e.g., at least about 95% relative to the deamidated versions of these peptides (i.e., contain less than 5% of the corresponding deamidated peptide). For instance, synthetic Gro-β, Gro-β T. and variants thereof that may be used in conjunction with the compositions and methods described herein may have a purity of about 95%, 95.5%, 96%, 96.5%, 97%, 97.5%, 98%, 98.5%, 99%, 99.5%, 99.6%, 99.7%, 99.8%, 99.9%, 99.99%, or more, relative to the deamidated versions of these peptides (e.g., the Asn69 deamidated version of SEQ ID NO: 1 or the Asn65 deamidated version of SEQ ID NO: 2). For instance, s\Synthetic Gro-β, Gro-β T, and variants thereof may have, for instance, a purity of from about 95% to about 99.99%, such as a purity of from about 95% to about 99.99%, about 96% to about 99.99%, about 97% to about 99.99%, about 98% to about 99.99%, about 99% to about 99.99%, about 99.9% to about 99.99%, about 95% to about 99.5%, about 96% to about 99.5%, about 95% to about 99%, or about 97% to about 99% relative to the deamidated versions of these peptides (e.g., the Asn69 deamidated version of SEQ ID NO: 1 or the Asn65 deamidated version of SEQ ID NO: 2).

Cell Population with Expanded Hematopoietic Stem Cells as Obtained by the Expansion Method and Therapeutic Compositions

In some aspects, the disclosure features a composition comprising a population of hematopoietic stem cells, wherein the hematopoietic stem cells or progenitors thereof have been contacted with the compound of any one of the above aspects or embodiments, thereby expanding the hematopoietic stem cells or progenitors thereof.

In some aspects, the present disclosure provides a cell population with expanded hemapoetic stem cells obtainable or obtained by the expansion method described above. In some embodiments, such cell population is resuspended in a pharmaceutically acceptable medium suitable for administration to a mammalian host, thereby providing a therapeutic composition.

The compound as defined in the present disclosure enables the expansion of HSCs, for example from only one or two cord blood units, to provide a cell population quantitatively and qualitatively appropriate for efficient short and long term engraftment in a human patient in need thereof. In some embodiments, the present disclosure relates to a therapeutic composition comprising a cell population with expanded HSCs derived from not more than one or two cord blood units. In some embodiments, the present disclosure relates to a therapeutic composition containing a total amount of cells of at least about 105, at least about 10⁶, at least about 10⁷, at least about 10⁸ or at least about 10⁹ cells with about 20% to about 100%, for example between about 43% to about 80%, of total cells being CD34+ cells. In certain embodiments, said composition contains between 20-100%, for example between 43-80%, of total cells being CD34+CD90+CD45RA−.

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoetic stem cells. In some embodiments, the hematopoietic stem cells are CD34+CD90+CD45RA− hematopoietic stem cells.

In some embodiments, the hematopoietic stem cells of the therapeutic composition are mammalian cells, such as human cells. In some embodiments, the human cells are CD34+ cells, such as CD34+ cells are CD34+, CD34+CD38−, CD34+CD38−CD90+, CD34+CD38−CD90+CD45RA−, CD34+CD38−CD90+CD45RA-CD49F+, or CD34+CD90+CD45RA− cells.

In some embodiments, the hematopoietic stem cells of the therapeutic composition are obtained from human cord blood, mobilized human peripheral blood, or human bone marrow. The hematopoietic stem cells may, for example, be freshly isolated from the human or may have been previously cryopreserved.

### Methods of Treatment

As described herein, hematopoietic stem cell transplant therapy can be administered to a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis. Thus, hematopoietic stem and progenitor cells represent a useful therapeutic modality for the treatment of a wide array of disorders in which a patient has a deficiency or defect in a cell type of the hematopoietic lineage. The deficiency or defect may be caused, for example, by depletion of a population of endogenous cells of the hematopoietic system due to administration of a chemotherapeutic agent (e.g., in the case of a patient suffering from a cancer, such as a hematologic cancer described herein). The deficiency or defect may be caused, for example, by depletion of a population of endogenous hematopoietic cells due to the activity of self-reactive immune cells, such as T lymphocytes or B lymphocytes that cross-react with self antigens (e.g., in the case of a patient suffering from an autoimmune disorder, such as an autoimmune disorder described herein). Additionally or alternatively, the deficiency or defect in cellular activity may be caused by aberrant expression of an enzyme (e.g., in the case of a patient suffering from various metabolic disorders, such as a metabolic disorder described herein).

Thus, hematopoietic stem cells can be administered to a patient defective or deficient in one or more cell types of the hematopoietic lineage in order to re-constitute the defective or deficient population of cells in vivo, thereby treating the pathology associated with the defect or depletion in the endogenous blood cell population. Hematopoietic stem and progenitor cells can be used to treat, e.g., a non-malignant hemoglobinopathy (e.g., a hemoglobinopathy selected from the group consisting of sickle cell anemia, thalassemia, Fanconi anemia, aplastic anemia, and Wiskott-Aldrich syndrome). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of cells that are damaged or deficient in the patient.

Hematopoietic stem or progenitor cells mobilized to the peripheral blood of a subject may be withdrawn (e.g., harvested or collected) from the subject by any suitable technique. For example, the hematopoietic stem or progenitor cells may be withdrawn by a blood draw. In some embodiments, hematopoietic stem or progenitor cells mobilized to a subject's peripheral blood as contemplated herein may be harvested (i.e., collected) using apheresis. In some embodiments, apheresis may be used to enrich a donors blood with mobilized hematopoietic stem or progenitor cells.

A dose of the expanded hematopoietic stem cell composition of the disclosure is deemed to have achieved a therapeutic benefit if it alleviates a sign or a symptom of the disease. The sign or symptom of the disease may comprise one or more biomarkers associated with the disease, or one or more clinical symptoms of the disease.

For example, administration of the expanded hematopoietic stem cell composition may result in the reduction of a biomarker that is elevated in individuals suffering from the disease, or elevate the level of a biomarker that is reduced in individuals suffering from the disease. Additionally or alternatively, hematopoietic stem and progenitor cells can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of immune cells (e.g., T lymphocytes, B lymphocytes, NK cells, or other immune cells) that are damaged or deficient in the patient.

Hematopoietic stem and progenitor cells can also be used to treat a metabolic disorder (e.g., a metabolic disorder selected from the group consisting of glycogen storage diseases, mucopolysaccharidoses, Gaucher disease, Hurler disease, sphingolipidoses, metachromatic leukodystrophy, globoid cell leukodystrophy, and cerebral adrenoleukodystrophy). In these cases, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of hematopoietic cells that are damaged or deficient in the patient.

Additionally or alternatively, hematopoietic stem or progenitor cells can be used to treat a malignancy or proliferative disorder, such as a hematologic cancer or myeloproliferative disease. In the case of cancer treatment, for example, a CXCR4 antagonist and/or a CXCR2 agonist may be administered to a donor, such as a donor identified as likely to exhibit release of a population of hematopoietic stem and progenitor cells from a stem cell niche, such as the bone marrow, into circulating peripheral blood in response to such treatment. The hematopoietic stem and progenitor cells thus mobilized may then be withdrawn from the donor and administered to a patient, where the cells may home to a hematopoietic stem cell niche and re-constitute a population of cells that are damaged or deficient in the patient, such as a population of hematopoietic cells that is damaged or deficient due to the administration of one or more chemotherapeutic agents to the patient. In some embodiments, hematopoietic stem or progenitor cells may be infused into a patient in order to repopulate a population of cells depleted during cancer cell eradication, such as during systemic chemotherapy. Exemplary hematological cancers that can be treated by way of administration of hematopoietic stem and progenitor cells in accordance with the compositions and methods described herein are acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated by the administration of hematopoietic stem and progenitor cells to a patient include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

In addition, administration of hematopoietic stem and progenitor cells can be used to treat autoimmune disorders. In some embodiments, upon infusion into a patient, transplanted hematopoietic stem and progenitor cells may home to a stem cell niche, such as the bone marrow, and establish productive hematopoiesis. This, in turn, can re-constitute a population of cells depleted during autoimmune cell eradication, which may occur due to the activity of self-reactive lymphocytes (e.g., self-reactive T lymphocytes and/or self-reactive B lymphocytes). Autoimmune diseases that can be treated by way of administering hematopoietic stem and progenitor cells to a patient include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pemicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter s syndrome, rheumatic fever, sarcoidosis, scleroderma. Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

Hematopoietic stem cell transplant therapy may additionally be used to treat neurological disorders, such as Parkinson's disease, Alzheimer's disease, multiple sclerosis, Amyotrophic lateral sclerosis, Huntington's disease, mild cognitive impairment, amyloidosis. AIDS-related dementia, encephalitis, stroke, head trauma, epilepsy, mood disorders, and dementia. As described herein, upon transplantation into a patient, hematopoietic stem cells may migrate to the central nervous system and differentiate into, for example, microglial cells, thereby re-constituting a population of cells that may be damaged or deficient in a patient suffering from a neurological disorder. In these cases, for example, a population of hematopoietic stem cells may be administered to a patient suffering from a neurological disorder, where the cells may home to the central nervous system, such as the brain of the patient, and re-constitute a population of hematopoietic cells (e.g., microglial cells) that are damaged or deficient in the patient.

### Selection of Donors and Patients

In some embodiments, the patient is the donor. In such cases, withdrawn hematopoietic stem or progenitor cells may be re-infused into the patient, such that the cells may subsequently home hematopoietic tissue and establish productive hematopoiesis, thereby populating or repopulating a line of cells that is defective or deficient in the patient (e.g., a population of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes). In this scenario, the transplanted hematopoietic stem or progenitor cells are least likely to undergo graft rejection, as the infused cells are derived from the patient and express the same HLA class I and class II antigens as expressed by the patient.

Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs).

In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Administration and Dosing of Hematopoietic Stem or Progenitor Cells

Hematopoietic stem and progenitor cells described herein may be administered to a subject, such as a mammalian subject (e.g., a human subject) suffering from a disease, condition, or disorder described herein, by one or more routes of administration. For instance, hematopoietic stem cells described herein may be administered to a subject by intravenous infusion. Hematopoietic stem cells may be administered at any suitable dosage. Non-limiting examples of dosages include about 1×10⁵ CD34+ cells/kg of recipient to about 1×10⁷ CD34+ cells/kg (e.g., from about 2×10⁵ CD34+ cells/kg to about 9×10⁶ CD34+ cells/kg, from about 3×10⁵ CD34+ cells/kg to about 8×10⁶ CD34+ cells/kg, from about 4×10⁵ CD34+ cells/kg to about 7×10⁶ CD34+ cells/kg, from about 5×10⁵ CD34+ cells/kg to about 6×10⁶ CD34+ cells/kg, from about 5×10⁵ CD34+ cells/kg to about 1×10⁷ CD34+ cells/kg, from about 6×10⁵ CD34+ cells/kg to about 1×10⁷ CD34+ cells/kg, from about 7×10⁵ CD34+ cells/kg to about 1×10⁷ CD34+ cells/kg, from about 8×10⁵ CD34+ cells/kg to about 1×10⁷ CD34+ cells/kg, from about 9×10⁵ CD34+ cells/kg to about 1×10⁷ CD34+ cells/kg, or from about 1×10⁶ CD34+ cells/kg to about 1×10⁷ CD34+ cells/kg, among others).

Hematopoietic stem or progenitor cells and pharmaceutical compositions described herein may be administered to a subject in one or more doses. When multiple doses are administered, subsequent doses may be provided one or more days, weeks, months, or years following the initial dose.

### Methods of Treating Inherited Metabolic Disorders—Administration of Expanded CD90+ Stem Cells for Microglial Engraftment in the Brain

As described herein, hematopoietic stem cell transplant therapy can be administered to a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages including, in some embodiments, microglia.

In some embodiments, hematopoietic stem cell transplant therapy or hematopoietic stem cell transplantation of inherited metabolic disorders may be accomplished using cross-correction. (Wynn, R. “Stem Cell Transplantation in Inherited Metabolic Disorders” Hematology 2011, pp. 285-291.) Cross correction involves engraftment of expanded HSCs in the patient or host tissue, where the implanted cells secrete the deficient enzyme and said deficient enzyme is then taken up by cells in the patient which are deficient in that enzyme.

In some embodiments, the inherited metabolic disorder to be treated is selected from Hurler syndrome (Hurler disease), mucopolysacchande disorders (e.g., Maroteaux Lamy syndrome), lysosomal storage disorders, and peroxisomal disorders (e.g., X-linked adrenoleukodystrophy), glycogen storage diseases, mucopolysaccharidoses, Mucolipidosis II, Gaucher disease, sphingolipidoses, metachromatic leukodystrophy, globoid cell leukodystrophy, and cerebral adrenoleukodystrophy.

In certain embodiments, HSCs in the patient or in a healthy donor are mobilized using a CXCR2 agonist and/or CXCR4 antagonist of the disclosure. The CXCR4 antagonist may be plerixafor or a variant thereof, and a CXCR2 agonist may be Gro-β or a variant thereof, such as a truncation of Gro-β, for instance, Gro-3 T. Mobilized HSCs are then isolated from a peripheral blood sample of the subject. Methods of isolating HSCs will be readily apparent to one of ordinary skill in the art. Alternatively, HSCs may be mobilized using a CXCR2 agonist and/or CXCR4 antagonist of the disclosure in a healthy individual who (1) does not suffer from an inherited metabolic disorder and (2) is a compatible donor for the subject who does suffer from the inherited metabolic disorder. HSCs can be isolated from a blood sample taken from this healthy individual collected following mobilization, the HSCs can then be expanded using the expansion methods of the disclosure, and the expanded cells transplanted into the subject with the inherited metabolic disorder.

It has been found that HSCs prepared with the methods of the disclosure lead to more microglia engraftment than fresh cells or cells cultured in the presence of cytokines. This is due to the presence of more CD90+ cells in expanded cell populations.

The methods disclosed herein for treating inherited metabolic disorders in a subject in need thereof comprise the administration of an expanded population of hematopoietic stem cells to a subject in need thereof. In some embodiments, the number of expanded hematopoietic stem cells administered to the subject is equal to or greater than the amount of hematopoietic stem cells needed to achieve a therapeutic benefit. In some embodiments, the number of expanded hematopoietic stem cells administered to the subject is greater than the amount of hematopoietic stem cells needed to achieve a therapeutic benefit. In some embodiments, the therapeutic benefit achieved is proportional to the number of expanded hematopoietic stem cells that are administered.

A dose of the expanded hematopoietic stem cell composition of the disclosure is deemed to have achieved a therapeutic benefit if it alleviates a sign or a symptom of the disease. The sign or symptom of the disease may comprise one or more biomarkers associated with the disease, or one or more clinical symptoms of the disease.

For example, administration of the expanded hematopoietic stem cell composition may result in the reduction of a biomarker that is elevated in individuals suffering from the disease, or elevate the level of a biomarker that is reduced in individuals suffering from the disease.

For example, administering the expanded hematopoietic stem cell composition of the disclosure may elevate the level of an enzyme that is reduced in an individual suffering from a metabolic disorder. This change in biomarker level may be partial, or the level of the biomarker may return to levels normally seen in healthy individuals.

In some embodiments, when the disease is, for example, an inherited metabolic disorder with a neurological component, the expanded hematopoietic stem cell composition may partly or fully reduce one or more clinical symptoms of the inherited metabolic disorder. Exemplary but non-limiting symptoms that may be affected by administration of the expanded hematopoietic stem cell composition of the disclosure comprise ataxias, dystonia, movement, disorders, epilepsies, and peripheral neuropathy.

In some cases, the sign or symptom of the inherited metabolic disorder with a neurological component comprises psychological signs or symptoms. For example, the sign or symptom of the disorder may comprise acute psychotic disorder, hallucinations, depressive syndrome, other symptoms or combinations of symptoms. Methods of evaluating psychological signs or symptoms associated with metabolic disorders with a neurological component will be known to one of ordinary skill in the art.

The onset of the inherited metabolic disorder may be adult or pediatric.

The inherited metabolic disorder may lead to degeneration of the nervous system.

Alleviating a sign or a symptom of the disorder may comprise slowing the rate of neurodegeneration or the rate of the progression of the disease.

Alleviating a sign or a symptom of the disorder may comprise reversing neurodegeneration or reversing the progression of the disease. Exemplary symptoms of neurodegeneration comprise memory loss, apathy, anxiety, agitation, loss of inhibition and mood changes. Methods of evaluating neurodegeneration, and the progression thereof, will be known to one of ordinary skill in the art. For example, in a patient suffering from Hurler syndrome, heparan and dermatan sulfate accumulation follows from α-L-iduronidase deficiency. Treatments that better clear these accumulated substrates will better correct the underlying disorder.

### Exemplary Embodiments

Embodiment No. 1: A method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents or reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

Embodiment No. 2: A method of administering expanded hematopoietic stem or progenitor cell transplant therapy to a patient in need thereof, the method comprising infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein the method prevents, or reduces the severity of, autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

Embodiment No. 3: A method of preventing, or reducing the risk of, autoimmune cytopenia in a patient in need thereof, the method comprising:

i) conditioning the patient with a conditioning regimen; and

ii) administering to the patient a population of hematopoietic stem or progenitor cells.

Embodiment No. 4: A method of preventing, or reducing the risk of, autoimmune cytopenia in a patient in need thereof, the method comprising:

i) conditioning the patient with a conditioning regimen; and

ii) transplanting the patient with expanded cord blood; wherein the conditioning regimen does not comprise busulfan plus fludarabine (BuFlu).

Embodiment No. 5: A method comprising administering to the patient a population of hematopoietic stem or progenitor cells, wherein the patient has previously been conditioned with a conditioning regimen.
Embodiment No. 6: A method of administering hematopoietic stem cell transplantation therapy to a patient in need thereof, wherein the patient has previously been conditioned with a conditioning regimen, the method comprising infusing into the patient a population of hematopoietic stem or progenitor cells.
Embodiment No. 7: A method comprising:

a) conditioning the patient with a conditioning regimen; and

b) administering to (e.g., infusing into) the patient a population of hematopoietic stem or progenitor cells.

Embodiment No. 8: A method of preventing or reducing the risk of autoimmune cytopenia in a patient in need thereof, the method comprising:

administering a prophylactic agent prior to, during, or following transplant with expanded cord blood; and

transplanting the patient with expanded cord blood;

wherein the prophylactic agent inhibits the production of antibodies in the patient.

Embodiment No. 9: A method of preventing or reducing the risk of autoimmune cytopenia in a patient in need thereof, the method comprising:

conditioning the patient with a conditioning regimen; and

transplanting the patient with expanded cord blood;

wherein the conditioning regimen is not busulfan plus fludarabine (BuFlu).

Embodiment No. 10: A method of administering hematopoietic stem or progenitor cell transplant therapy to a patient in need thereof, the method comprising:

(a) conditioning the patient with a conditioning regimen; and

(b) infusing into the patient a population of hematopoietic stem or progenitor cells.

Embodiment No. 11: A method of preparing a patient for hematopoietic stem or progenitor cell transplantation, the method comprising conditioning the patient with a conditioning regimen.
Embodiment No. 12: A method of administering hematopoietic stem cell transplantation therapy to a patient in need thereof, wherein the patient has previously been conditioned with a conditioning regimen, the method comprising infusing into the patient a population of hematopoietic stem or progenitor cells.
Embodiment No. 13: A method of administering hematopoietic stem or progenitor cell transplant therapy to a patient in need thereof, the method comprising:

(a) conditioning the patient with a conditioning regimen, wherein the conditioning regimen comprising
(a1) administering busulfan (Bu);
(a2) administering cyclophosphamide (Cy); and
(a3) administering anti-thymocyte globulin (rabbit)(ATG); and

(b) infusing into the patient a population of hematopoietic stem or progenitor cells.

Embodiment No. 14: A method of administering hematopoietic stem or progenitor cell transplant therapy to a patient in need thereof, the method comprising:

(a) conditioning the patient with a conditioning regimen, wherein the conditioning regimen comprising
(a1) administering busulfan (Bu) prior to administering cyclophosphamide and prior to administering anti-thymocyte globulin (rabbit) (ATG);
(a2) administering cyclophosphamide (Cy) after administering busulfan (Bu) and simultaneously with administering anti-thymocyte globulin (rabbit) (ATG); and
(a3) administering anti-thymocyte globulin (rabbit) (ATG) after administering busulfan (Bu) and simultaneously with administering cyclophosphamide (Cy); and

(b) infusing into the patient a population of hematopoietic stem or progenitor cells.

Embodiment No. 15: A method of administering hematopoietic stem or progenitor cell transplant therapy to a patient in need thereof, the method comprising:

(a) conditioning the patient with a conditioning regimen, wherein the conditioning regimen comprising
(a1) administering busulfan (Bu) at days −9 to −6 prior to infusing into the patient a population of hematopoietic stem or progenitor cells;
(a2) administering cyclophosphamide (Cy) at days −5 to −2 prior to infusing into the patient a population of hematopoietic stem or progenitor cells; and
(a3) administering anti-thymocyte globulin (rabbit)(ATG) at days −5 to −2 prior to infusing into the patient a population of hematopoietic stem or progenitor cells; and

(b) infusing into the patient a population of hematopoietic stem or progenitor cells at day 0.

Embodiment No. 16: A method of administering hematopoietic stem or progenitor cell transplant therapy to a patient in need thereof, the method comprising:

(a) conditioning the patient with a conditioning regimen, wherein the conditioning regimen comprising
(a1) administering busulfan (Bu) at a dose wherein the plasma exposure as measured by cumulative AUC is maintained within a range of 74-82 mg*hr/L for 4 consecutive days at days −9 to −6 prior to infusing into the patient a population of hematopoietic stem or progenitor cells;
(a2) administering cyclophosphamide (Cy) at a dose of about 50 mg/kg/day for 4 consecutive days at days −5 to −2 prior to infusing into the patient a population of hematopoietic stem or progenitor cells; and
(a3) administering anti-thymocyte globulin (rabbit)(ATG) at a dose of about 2.5 mg/kg/day for 4 consecutive days at days −5 to −2 prior to infusing into the patient a population of hematopoietic stem or progenitor cells; and

(b) infusing into the patient a population of hematopoietic stem or progenitor cells at day 0.

Embodiment No. 17: A method of administering hematopoietic stem or progenitor cell transplant therapy to a patient in need thereof, the method comprising:

(a) conditioning the patient with a conditioning regimen, wherein the conditioning regimen comprising
(a1) administering busulfan (Bu) at a dose wherein the plasma exposure as measured by steady state concentration (Css) is maintained within a range of 770-850 ng/mL for 4 consecutive days at days −9 to −6 prior to infusing into the patient a population of hematopoietic stem or progenitor cells;
(a2) administering cyclophosphamide (Cy) at a dose of about 50 mg/kg/day for 4 consecutive days at days −5 to −2 prior to infusing into the patient a population of hematopoietic stem or progenitor cells; and
(a3) administering anti-thymocyte globulin (rabbit)(ATG) at a dose of about 2.5 mg/kg/day for 4 consecutive days at days −5 to −2 prior to infusing into the patient a population of hematopoietic stem or progenitor cells; and

(b) infusing into the patient a population of hematopoietic stem or progenitor cells at day 0.

Embodiment No. 18: A method of administering hematopoietic stem or progenitor cell transplant therapy to a patient in need thereof in accordance with the method of any one of the preceding embodiments, the method comprising:

(a) expanding, ex vivo, a population of CD34+ cells comprising no more than 1×10⁸ CD34+ cells; and

(b) infusing into the patient the hematopoietic stem or progenitor cells, or progeny thereof, expanded in (a).

Embodiment No. 19: A method of administering hematopoietic stem or progenitor cell transplant therapy to a patient in need thereof in accordance with the method of any one of the preceding embodiments, the method comprising infusing into the patient a population of hematopoietic stem or progenitor cells that have been expanded ex vivo, wherein the population, prior to expansion, comprises no more than 1×10⁸ CD34+ cells.
Embodiment No. 20: A method of treating a stem cell disorder in a patient, the method comprising administering hematopoietic stem or progenitor cell transplant therapy to the patient in accordance with the method of any one of the preceding embodiments.
Embodiment No. 21: A population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the risk of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

Embodiment No. 22: A population of expanded hematopoietic stem or progenitor cells for administering expanded hematopoietic stem or progenitor cell transplant therapy to a patient in need thereof, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);

wherein autoimmune cytopenia is prevented, or the severity of autoimmune cytopenia is reduced, in the patient as compared to a patient conditioned prior to receiving the population of expanded hematopoietic stem or progenitor cells by a conditioning regimen comprising administering to the patient busulfan (Bu) and fludarabine (Flu).

Embodiment No. 23: A combination of a conditioning regimen and a population of hematopoietic stem or progenitor cells for preventing, or reducing the risk of, autoimmune cytopenia in a patient in need thereof, wherein the patient is conditioned with the conditioning regimen prior to being administered with the population of hematopoietic stem or progenitor cells.
Embodiment No. 24: A combination of a conditioning regimen and an expanded cord blood for preventing, or reducing the risk of, autoimmune cytopenia in a patient in need thereof, wherein the patient is conditioned with the conditioning regimen prior to being administered with the expanded cord blood, and wherein the conditioning regimen does not comprise busulfan plus fludarabine (BuFlu).
Embodiment No. 25: A population of hematopoietic stem or progenitor cells for being administered to a patient, wherein the patient is conditioned with a conditioning regimen prior to the administration of the population of hematopoietic stem or progenitor cells.
Embodiment No. 26: A population of hematopoietic stem or progenitor cells for administering hematopoietic stem cell transplantation therapy to a patient in need thereof, wherein the is conditioned with a conditioning regimen prior to infusing into the patient the population of hematopoietic stem or progenitor cells.
Embodiment No. 27: A conditioning regimen (e.g., prophylactic agent) for preventing or reducing the risk of autoimmune cytopenia in a patient in need thereof, wherein the conditioning regimen (e.g., prophylactic agent) is administered to the patient prior to, during, or following transplanting the patient with expanded cord blood; and wherein the conditioning regimen (e.g., prophylactic agent) inhibits the production of antibodies in the patient.
Embodiment No. 28: A conditioning regimen for preparing a patient for hematopoietic stem or progenitor cell transplantation.
Embodiment No. 29: A population of hematopoietic stem or progenitor cells for administering hematopoietic stem or progenitor cell transplant therapy to a patient in need thereof, wherein the population of hematopoietic stem or progenitor cells that have been expanded ex vivo, wherein the population, prior to expansion, comprises no more than 1×10⁸ CD34+ cells.
Embodiment No. 30: A population of hematopoietic stem or progenitor cells for treating a stem cell disorder in a patient.
Embodiment No. 31: The method, conditioning regimen, population, or combination of any one of the preceding embodiments, wherein the expanded cord blood has been expanded with an aryl hydrocarbon receptor antagonist.
Embodiment No. 32: The method, conditioning regimen, population, or combination of any one of the preceding embodiments, wherein the conditioning regimen comprises a prophylactic agent.
Embodiment No. 33: The method, conditioning regimen, population, or combination of any one of the preceding embodiments, wherein the prophylactic agent is an anti-CD20 antibody.
Embodiment No. 34: The method, conditioning regimen, population, or combination of any one of the preceding embodiments, wherein the anti-CD20 antibody is rituximab.
Embodiment No. 35: The method, conditioning regimen, population, or combination of any one of the preceding embodiments, wherein the prophylactic agent is administered in combination with intravenous immunoglobulin (IVIG).
Embodiment No. 36: The method, conditioning regimen, population, or combination of any one of the preceding embodiments, wherein the expanded cord blood has been expanded with an aryl hydrocarbon receptor antagonist.
Embodiment No. 37: The method, conditioning regimen, population, or combination of any one of the preceding embodiments, wherein the conditioning regimen is busulfan plus cyclophosphamide (BuCy).
Embodiment No. 38: The method, conditioning regimen, population, or combination of any one of the preceding embodiments, wherein the conditioning regimen substantially ablates the patient's B-cells.
Embodiment No. 39: The method, conditioning regimen, population, or combination of any one of the preceding embodiments, wherein the expanded cord blood is MGTA-456.
Embodiment No. 40: The method, conditioning regimen, population, or combination of any one of the preceding embodiments, wherein the patient is 2 years old or younger.
Embodiment No. 41: The method, conditioning regimen, population, or combination of any one of the preceding embodiments, wherein the patient has an inherited metabolic disorder.
Embodiment No. 42: The method, conditioning regimen, population, or combination of any one of the preceding embodiments, wherein the inherited metabolic disorder is Hurler disease, metachromatic leukodystrophy, globoid cell leukodystrophy, or cerebral adrenoleukodystrophy.
Embodiment No. 43: The method, conditioning regimen, population, or combination of any one of the preceding embodiments, wherein the conditioning regimen comprises administering radiation and/or a prophylactic agent to the patient.
Embodiment No. 44: The method, conditioning regimen, population, or combination of any one of the preceding embodiments, wherein the conditioning regimen is administered prior to, during, or following the infusion of a population of stem or progenitor cells.
Embodiment No. 45: The method, conditioning regimen, population, or combination of any one of the preceding embodiments, wherein the conditioning regimen is administered prior to the infusing of a population of hematopoietic stem or progenitor cells.
Embodiment No. 46: The method, conditioning regimen, population, or combination of any one of the preceding embodiments, wherein the prophylactic agent is an anti-CD20 antibody.
Embodiment No. 47: The method, conditioning regimen, population, or combination of any one of the preceding embodiments, wherein the prophylactic agent is rituximab.
Embodiment No. 48: The method, conditioning regimen, population, or combination of any one of the preceding embodiments, wherein the conditioning regimen comprises administering one or more agents selected from the group consisting of an alkylating agent and an anti-leukocyte globulin.
Embodiment No. 49: The method, conditioning regimen, population, or combination of any one of the preceding embodiments, wherein the conditioning regimen comprises administering at least one alkylating agent and at least one anti-leukocyte globulin simultaneously, sequentially, or in alteration.
Embodiment No. 50: The method, conditioning regimen, population, or combination of any one of the preceding embodiments, wherein the conditioning regimen comprises administering a first alkylating agent, a second alkylating agent, and an anti-leukocyte globulin simultaneously, sequentially, or in alteration.
Embodiment No. 51: The method, conditioning regimen, population, or combination of any one of the preceding embodiments, wherein the conditioning regimen comprises administering the first alkylating agent prior to the anti-leukocyte globulin and the second alkylating agent simultaneously with the anti-leukocyte globulin.
Embodiment No. 52: The method, conditioning regimen, population, or combination of any one of the preceding embodiments, wherein the anti-leukocyte globulin is an anti-thymocyte globulin (ATG).
Embodiment No. 53: The method, conditioning regimen, population, or combination of any one of the preceding embodiments, wherein the anti-thymocyte globulin is anti-thymocyte globulin (rabbit) (Thymoglobulin, ATG) and/or anti-thymocyte (equine) (Atgam).
Embodiment No. 54: The method, conditioning regimen, population, or combination of any one of the preceding embodiments, wherein the anti-thymocyte globulin is anti-thymocyte globulin (rabbit) (Thymoglobulin, ATG).
Embodiment No. 55: The method, conditioning regimen, population, or combination of any one of the preceding embodiments, wherein the alkylating agent is busulfan (Bu) and/or cyclophosphamide (Cy).
Embodiment No. 56: The method, conditioning regimen, population, or combination of any one of the preceding embodiments, wherein the alkylating agent is busulfan (Bu).
Embodiment No. 57: The method, conditioning regimen, population, or combination of any one of the preceding embodiments, wherein the first alkylating agent is busulfan (Bu).
Embodiment No. 58: The method, conditioning regimen, population, or combination of any one of the preceding embodiments, wherein the alkylating agent is cyclophosphamide (Cy).
Embodiment No. 59: The method, conditioning regimen, population, or combination of any one of the preceding embodiments, wherein the second alkylating agent is cyclophosphamide (Cy).
Embodiment No. 60: The method, conditioning regimen, population, or combination of any one of the preceding embodiments, wherein the first alkylating agent is busulfan (Bu) and the second alkylating agent is cyclophosphamide (Cy).
Embodiment No. 61: The method, conditioning regimen, population, or combination of any one of the preceding embodiments, wherein the conditioning regimen comprises administering busulfan (Bu), cyclophosphamide (Cy) and anti-thymocyte globulin (rabbit) (Thymoglobulin, ATG) simultaneously, sequentially, or in alteration.
Embodiment No. 62: The method, conditioning regimen, population, or combination of any one of the preceding embodiments, wherein the conditioning regimen comprises administering busulfan (Bu) prior to anti-thymocyte globulin (rabbit) (Thymoglobulin, ATG) and cyclophosphamide (Cy) simultaneously with anti-thymocyte globulin (rabbit) (Thymoglobulin, ATG).
Embodiment No. 63: The method, conditioning regimen, population, or combination of any one of the preceding embodiments, wherein the busulfan (Bu) is administered intravenously.
Embodiment No. 64: The method, conditioning regimen, population, or combination of any one of the preceding embodiments, wherein the busulfan (Bu) is administered at a dose wherein the plasma exposure as measured by cumulative AUC is maintained within a range of 74-82 mg*hr/L.
Embodiment No. 65: The method, conditioning regimen, population, or combination of any one of the preceding embodiments, wherein the busulfan (Bu) is administered at a dose wherein the plasma exposure as measured by cumulative AUC is maintained within at about 78 mg*hr/L.
Embodiment No. 88: The method, conditioning regimen, population, or combination of any one of the preceding embodiments, wherein the busulfan (Bu) is administered at a dose wherein the plasma exposure as measured by steady state concentration (Cₛₛ) is maintained within a range of 770-850 ng/mL.
Embodiment No. 67: The method, conditioning regimen, population, or combination of any one of the preceding embodiments, wherein the busulfan (Bu) is administered at a dose wherein the plasma exposure as measured by steady state concentration (Cₛₛ) is maintained at about 810 ng/mL.
Embodiment No. 68: The method, conditioning regimen, population, or combination of any one of the preceding embodiments, wherein the busulfan (Bu) is administered in a total of 4 doses.
Embodiment No. 69: The method, conditioning regimen, population, or combination of any one of the preceding embodiments, wherein the busulfan (Bu) is administered in a total of 4 doses once daily.
Embodiment No. 70: The method, conditioning regimen, population, or combination of any one of the preceding embodiments, wherein the busulfan (Bu) is administered in a total of 4 doses once daily over a time period of about 3 hours per dose.
Embodiment No. 71: The method, conditioning regimen, population, or combination of any one of the preceding embodiments, wherein the busulfan (Bu) is administered in a total of 4 doses once daily with an initial dose in a range of about 80 mg/m² to about 120 mg/m².
Embodiment No. 72: The method, conditioning regimen, population, or combination of any one of the preceding embodiments, wherein the busulfan (Bu) is administered in a total of 16 doses.
Embodiment No. 73: The method, conditioning regimen, population, or combination of any one of the preceding embodiments, wherein the busulfan (Bu) is administered in a total of 16 doses every 6 hours.
Embodiment No. 74: The method, conditioning regimen, population, or combination of any one of the preceding embodiments, wherein the busulfan (Bu) is administered in a total of 16 doses every 6 hours over a time period of about 2 hours per dose.
Embodiment No. 75: The method, conditioning regimen, population, or combination of any one of the preceding embodiments, wherein the busulfan (Bu) is administered in a total of 16 doses every 6 hours with an initial dose of about 1 mg/kg.
Embodiment No. 76: The method, conditioning regimen, population, or combination of any one of the preceding embodiments, wherein the busulfan (Bu) is administered for about 4 consecutive days.
Embodiment No. 77: The method, conditioning regimen, population, or combination of any one of the preceding embodiments, wherein the busulfan (Bu) is administered for 4 consecutive days at days −9 to −6 prior to infusing into the patient a population of hematopoietic stem or progenitor cells.
Embodiment No. 78: The method, conditioning regimen, population, or combination of any one of the preceding embodiments, wherein the cyclophosphamide (Cy) is administered intravenously.
Embodiment No. 79: The method, conditioning regimen, population, or combination of any one of the preceding embodiments, wherein the cyclophosphamide (Cy) is administered at a dosage of about 50 mg/kg/day.
Embodiment No. 80: The method, conditioning regimen, population, or combination of any one of the preceding embodiments, wherein the daily dosage of about 50 mg/kg/day of the cyclophosphamide (Cy) is administered over a time period of about 1 hour per dose.
Embodiment No. 81: The method, conditioning regimen, population, or combination of any one of the preceding embodiments, wherein the cyclophosphamide (Cy) is administered for about 4 consecutive days.
Embodiment No. 82: The method, conditioning regimen, population, or combination of any one of the preceding embodiments, wherein the cyclophosphamide (Cy) is administered for 4 consecutive days at days −5 to −2 prior to infusing into the patient a population of hematopoietic stem or progenitor cells.
Embodiment No. 83: The method, conditioning regimen, population, or combination of any one of the preceding embodiments, wherein the first dose of the cyclophosphamide (Cy) is administered at least 24 hours after the last dose of busulfan (Bu).
Embodiment No. 84: The method, conditioning regimen, population, or combination of any one of the preceding embodiments, wherein the anti-thymocyte globulin (rabbit) (Thymoglobulin, ATG) is administered intravenously.
Embodiment No. 85: The method, conditioning regimen, population, or combination of any one of the preceding embodiments, wherein the anti-thymocyte globulin (rabbit) (Thymoglobulin, ATG) is administered at a dosage of about 2.5 mg/kg/day.
Embodiment No. 86: The method, conditioning regimen, population, or combination of any one of the preceding embodiments, wherein the daily dosage of about 2.5 mg/kg/day of the anti-thymocyte globulin (rabbit) (Thymoglobulin, ATG) is administered over a time period of about 2 hours to about 10 hours.
Embodiment No. 87: The method, conditioning regimen, population, or combination of any one of the preceding embodiments, wherein the daily dosage of about 2.5 mg/kg/day of the anti-thymocyte globulin (rabbit) (Thymoglobulin, ATG) is administered over a time period of about 6 hours per dose.
Embodiment No. 88: The method, conditioning regimen, population, or combination of any one of the preceding embodiments, wherein the anti-thymocyte globulin (rabbit) (Thymoglobulin, ATG) is administered for about 4 consecutive days.
Embodiment No. 89: The method, conditioning regimen, population, or combination of any one of the preceding embodiments, wherein the anti-thymocyte globulin (rabbit) (Thymoglobulin, ATG) is administered for 4 consecutive days at days −5 to −2 prior to infusing into the patient a population of hematopoietic stem or progenitor cells.
Embodiment No. 90: The method, conditioning regimen, population, or combination of any one of the preceding embodiments, wherein upon transplantation, the risk of autoimmune cytopenia is prevented or reduced in the patient relative to a patient that is administered a conditioning regimen comprising busulfan and fludarabine (BuFlu) prior to transplantation.
Embodiment No. 91: The method, conditioning regimen, population, or combination of any one of the preceding embodiments, wherein upon transplantation, the risk of autoimmune cytopenia with an onset of at least 2 days, at least 5 days, at least 10 days, at least 20 days, at least 25 days, at least 50 days, at least 75 days, at least 100 days, at least 125 days, at least 150 days, at least 175 days, at least 200 days, at least 250 days, at least 300 days, or at least 350 days following the infusing into the patient of a population of hematopoietic stem or progenitor cells is prevented or reduced in the patient relative to a patient that is administered a conditioning regimen comprising busulfan and fludarabine (BuFlu) prior to transplantation.
Embodiment No. 92: The method, conditioning regimen, population, or combination of any one of the preceding embodiments, wherein the patient has an inherited metabolic disorder.
Embodiment No. 93: The method, conditioning regimen, population, or combination of any one of the preceding embodiments, wherein the inherited metabolic disorder is Hurler disease, metachromatic leukodystrophy, gioboid cell leukodystrophy, or cerebral adrenoleukodystrophy.
Embodiment No. 94: The method, conditioning regimen, population, or combination of any one of the preceding embodiments, wherein the hematopoietic stem or progenitor cells, or progeny thereof, maintain hematopoietic stem cell functional potential after 2 or more days following infusion of the hematopoietic stem or progenitor cells into the patient.
Embodiment No. 95: The method, conditioning regimen, population, or combination of any one of the preceding embodiments, wherein the hematopoietic stem or progenitor cells, or progeny thereof, localize to hematopoietic tissue and/or reestablish hematopoiesis following infusion of the hematopoietic stem or progenitor cells into the patient.
Embodiment No. 96: The method, conditioning regimen, population, or combination of any one of the preceding embodiments, wherein upon infusion into the patient, the hematopoietic stem or progenitor cells give rise to recovery of a population of cells selected from the group consisting of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.
Embodiment No. 97: The method, conditioning regimen, population, or combination of any one of the preceding embodiments, wherein the conditioning regimen comprises administering radiation and/or a prophylactic agent to the patient.
Embodiment No. 98: The method, conditioning regimen, population, or combination of any one of the preceding embodiments, wherein the conditioning regimen is administered prior to, during, or following the infusion of a population of stem or progenitor cells.
Embodiment No. 99: The method, conditioning regimen, population, or combination of any one of the preceding embodiments, wherein the prophylactic agent is an anti-CD20 antibody.
Embodiment No. 100: The method, conditioning regimen, population, or combination of any one of the preceding embodiments, wherein the prophylactic agent is rituximab.
Embodiment No. 101: The method, conditioning regimen, population, or combination of any one of the preceding embodiments, wherein the prophylactic agent is administered in combination with intravenous immunoglobulin (IVIG).
Embodiment No. 102 The method, conditioning regimen, population, or combination of any one of the preceding embodiments, wherein the conditioning regimen does not comprise busulfan plus fludarabine (BuFlu).
Embodiment No. 103: The method, conditioning regimen, population, or combination of any one of the preceding embodiments, wherein the conditioning regimen comprises busulfan plus cyclophosphamide (BuCy).
Embodiment No. 104: The method, conditioning regimen, population, or combination of any one of the preceding embodiments, wherein the conditioning regimen substantially ablates the B cells of the patient.
Embodiment No. 105: The method, conditioning regimen, population, or combination of any one of the preceding embodiments, wherein the conditioning regimen ablates at least 50% of the B cells of the patient, at least 60% of the B cells of the patient, at least 70% of the B cells of the patient, at least 75% of the B cells of the patient, at least 80% of the B cells of the patient, at least 85% of the B cells of the patient, at least 90% of the B cells of the patient, at least 95% of the B cells of the patient, or at least 98% of the B cells of the patient.
Embodiment No. 106: The method, conditioning regimen, population, or combination of any one of the preceding embodiments, wherein the patient is 2 years old or younger.
Embodiment No. 107: The method, conditioning regimen, population, or combination of any one of the preceding embodiments, further comprising administering a prophylactic agent against seizures prior to, during, or following the administering of busulfan (Bu).
Embodiment No. 108: The method, conditioning regimen, population, or combination of any one of the preceding embodiments, wherein the prophylactic agent against seizures is levetiracetam (Keppra).
Embodiment No. 109: The method, conditioning regimen, population, or combination of any one of the preceding embodiments, wherein the first dose of the prophylactic agent against seizures is administered at least about 12-24 hours prior to the first dose of busulfan (Bu) is administered and the last dose of the prophylactic agent against seizures is administered at least about 24 hours after the last dose of busulfan (Bu) is administered.
Embodiment No. 110: The method, conditioning regimen, population, or combination of any one of the preceding embodiments, further comprising administering a chemotherapy adjuvant prior to, during, or following the administering of cyclophosphamide.
Embodiment No. 111: The method, conditioning regimen, population, or combination of any one of the preceding embodiments, wherein the chemotherapy adjuvant is mesna (Mesnex).
Embodiment No. 112: The method, conditioning regimen, population, or combination of any one of the preceding embodiments, wherein the chemotherapy adjuvant is administered during the administering of cyclophosphamide.
Embodiment No. 113: The method, conditioning regimen, population, or combination of any one of the preceding embodiments, wherein the chemotherapy adjuvant is administered during the administering of cyclophosphamide at days −5 to −2 prior to infusing into the patient a population of hematopoietic stem or progenitor cells.
Embodiment No. 114: The method, conditioning regimen, population, or combination of any one of the preceding embodiments, further comprising administering an immunosuppression regimen to the patient.
Embodiment No. 115: The method, conditioning regimen, population, or combination of any one of the preceding embodiments, wherein the immunosuppression regimen comprises administering at least one immunosuppressant agent.
Embodiment No. 116: The method, conditioning regimen, population, or combination of any one of the preceding embodiments, wherein the immunosuppressant agent is administered prior to, during, or following the conditioning regimen.
Embodiment No. 117: The method, conditioning regimen, population, or combination of any one of the preceding embodiments, wherein the immunosuppressant agent is administered during and following the conditioning regimen.
Embodiment No. 118: The method, conditioning regimen, population, or combination of any one of the preceding embodiments, wherein the immunosuppressant agent is mycophenolate mofetil (MMF, CellCept), cyclosporine A (CsA), and/or salts or prodrugs thereof.
Embodiment No. 119: The method, conditioning regimen, population, or combination of any one of the preceding embodiments, wherein the immunosuppressant agent is mycophenolate mofetil (MMF).
Embodiment No. 120: The method, conditioning regimen, population, or combination of any one of the preceding embodiments, wherein the immunosuppressant agent is cyclosporine A (CsA).
Embodiment No. 121: The method, conditioning regimen, population, or combination of any one of the preceding embodiments, wherein the immunosuppression regimen comprises administering mycophenolate mofetil (MMF) and cyclosporine A (CsA).
Embodiment No. 122: The method, conditioning regimen, population, or combination of any one of the preceding embodiments, wherein the immunosuppression regimen comprises administering mycophenolate mofetil (MMF) and cyclosporine A (CsA) starting at day −3 prior to infusing into the patient a population of hematopoietic stem or progenitor cells.
Embodiment No. 123: The method, conditioning regimen, population, or combination of any one of the preceding embodiments, wherein cyclosporine A (CsA) is administered for at a dose wherein the serum trough level is maintained within a range of about 200-400 ng/mL.
Embodiment No. 124: The method, conditioning regimen, population, or combination of any one of the preceding embodiments, wherein cyclosporine A (CsA) is administered for at least 200 days following the infusion of a population of hematopoietic stem or progenitor cells.
Embodiment No. 125: The method, conditioning regimen, population, or combination of any one of the preceding embodiments, wherein mycophenolate mofetil (MMF) is administered intravenously or orally.
Embodiment No. 126: The method, conditioning regimen, population, or combination of any one of the preceding embodiments, wherein mycophenolate mofetil (MMF) is administered for about three times daily for at least 40 days following the infusion of a population of hematopoietic stem or progenitor cells.
Embodiment No. 127: The method, conditioning regimen, population, or combination of any one of the preceding embodiments, wherein mycophenolate mofetil (MMF) is administered at a dose of about 300 mg/kg/day to about 3000 mg/kg/day.
Embodiment No. 128: The method, conditioning regimen, population, or combination of any one of the preceding embodiments, wherein the method further comprises administering granulocyte colony-stimulating factor (G-CSF) prior to, during, or following the infusion of a population of hematopoietic stem or progenitor cells.
Embodiment No. 129: The method, conditioning regimen, population, or combination of any one of the preceding embodiments, wherein the granulocyte colony-stimulating factor (G-CSF) is administered following the infusion of a population of hematopoietic stem or progenitor cells.
Embodiment No. 130: The method, conditioning regimen, population, or combination of any one of the preceding embodiments, wherein the granulocyte colony-stimulating factor (G-CSF) is administered starting at day +1 following the infusion of a population of hematopoietic stem or progenitor cells.
Embodiment No. 131: The method, conditioning regimen, population, or combination of any one of the preceding embodiments, wherein the granulocyte colony-stimulating factor (G-CSF) is administered starting at day +1 following the infusion of a population of hematopoietic stem or progenitor cells until an absolute neutrophil count (ANC) is greater than or equal to about 2,500/μL for at least 2 consecutive days.
Embodiment No. 132: The method, conditioning regimen, population, or combination of any one of the preceding embodiments, wherein the hematopoietic stem or progenitor cells are expanded ex vivo prior to infusion into the patient.
Embodiment No. 133: The method, conditioning regimen, population, or combination of any one of the preceding embodiments, wherein the hematopoietic stem or progenitor cells are expanded ex vivo prior to infusion into the patient by contacting the hematopoietic stem or progenitor cells with at least one agent selected from the group consisting of an aryl hydrocarbon receptor antagonist, nicotinamide. UM729, and UM171.
Embodiment No. 134: The method, conditioning regimen, population, or combination of any one of the preceding embodiments, wherein the expanded cord blood or population of hematopoietic stem or progenitor cells is selected from the group consisting of MGTA-456, omidubicel (NiCord), and ECT-001.
Embodiment No. 135: The method, conditioning regimen, population, or combination of any one of the preceding embodiments, wherein the expanded cord blood or population of hematopoietic stem or progenitor cells is MGTA-456.
Embodiment No. 136: The method, conditioning regimen, population, or combination of any one of the preceding embodiments, wherein the hematopoietic stem or progenitor cells are expanded ex vivo by contacting the hematopoietic stem or progenitor cells with an aryl hydrocarbon receptor antagonist.
Embodiment No. 137: The method, conditioning regimen, population, or combination of any one of the preceding embodiments, wherein the aryl hydrocarbon receptor antagonist is SR-1 or a pharmaceutically acceptable salt, hydrate, or solvate thereof.
Embodiment No. 138: The method, conditioning regimen, population, or combination of any one of the preceding embodiments, wherein the aryl hydrocarbon receptor antagonist is compound 2 or a pharmaceutically acceptable salt, hydrate, or solvate thereof.
Embodiment No. 139: The method, conditioning regimen, population, or combination of any one of the preceding embodiments, wherein the aryl hydrocarbon receptor antagonist is a compound represented by formula (IV)

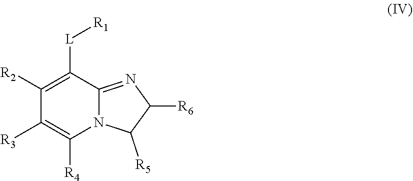

or a pharmaceutically acceptable salt, hydrate, or solvate thereof, wherein:

L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₈ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})_{f}—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR_{9b}, —C(O)NR₉ₐR_{9b}, —C(S)NR₉ₐR_{9b}, —NR₉ₐS(O)₂R_{9b}, —NR₉ₐC(O)OR_{9b}, —OC(O)CR₉ₐR_{9b}R_{9c}, —OC(S)NR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;

R₂ is selected from the group consisting of hydrogen and optionally substituted C1-4 alkyl;

R₃ is selected from the group consisting of optionally substituted aryl, optionally substituted heteroaryl, optionally substituted cycloalkyl, and optionally substituted heterocycloalkyl;

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

Embodiment No. 140: The method, conditioning regimen, population, or combination of any one of the preceding embodiments, wherein the aryl hydrocarbon receptor antagonist is compound (3)

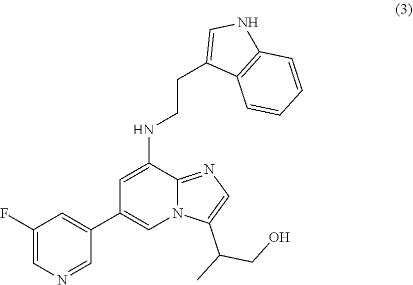 [SMILES: CC(CO)c1cnc2c(NCCc3c[nH]c4ccccc34)cc(-c3cncc(F)c3)cn12]

or a pharmaceutically acceptable salt, hydrate, or solvate thereof.
Embodiment No. 141: The method, conditioning regimen, population, or combination of any one of the preceding embodiments, wherein the aryl hydrocarbon receptor antagonist is compound (4)

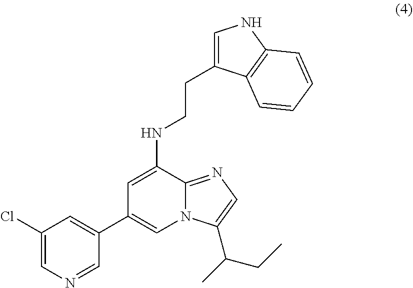 [SMILES: CCC(C)c1cnc2c(NCCc3c[nH]c4ccccc34)cc(-c3cncc(Cl)c3)cn12]

or a pharmaceutically acceptable salt, hydrate, or solvate thereof.
Embodiment No. 142. The method, conditioning regimen, population, or combination of any one of the preceding embodiments, wherein the aryl hydrocarbon receptor antagonist is compound (5)

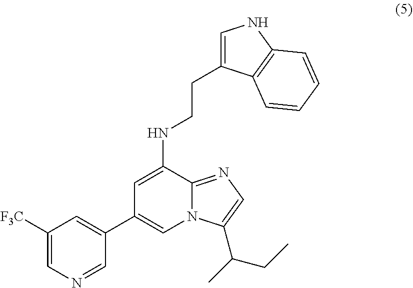 [SMILES: CCC(C)c1cnc2c(NCCc3c[nH]c4ccccc34)cc(-c3cncc(C(F)(F)F)c3)cn12]

or a pharmaceutically acceptable salt thereof.
Embodiment No. 143: The method, conditioning regimen, population, or combination of any one of the preceding embodiments, wherein the aryl hydrocarbon receptor antagonist is compound (6)

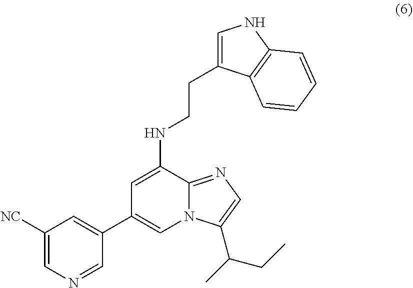 [SMILES: CCC(C)c1cnc2c(NCCc3c[nH]c4ccccc34)cc(-c3cncc(C#N)c3)cn12]

or a pharmaceutically acceptable salt, hydrate, or solvate thereof.
Embodiment No. 144: The method, conditioning regimen, population, or combination of any one of the preceding embodiments, wherein the aryl hydrocarbon receptor antagonist is compound (7)

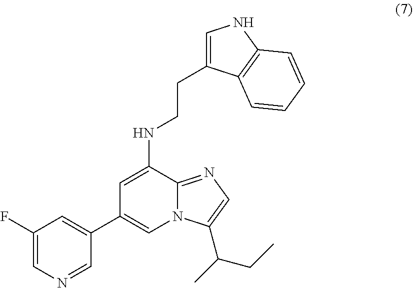 [SMILES: CCC(C)c1cnc2c(NCCc3c[nH]c4ccccc34)cc(-c3cncc(F)c3)cn12]

or a pharmaceutically acceptable salt, hydrate, or solvate thereof.
Embodiment No. 145: The method, conditioning regimen, population, or combination of any one of the preceding embodiments, wherein the aryl hydrocarbon receptor antagonist is compound (8)

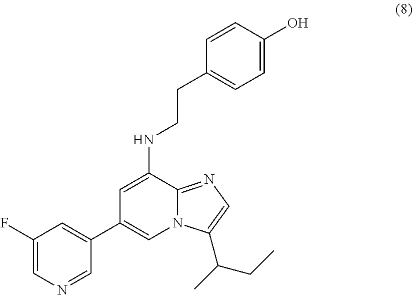 [SMILES: CCC(C)c1cnc2c(NCCc3ccc(O)cc3)cc(-c3cncc(F)c3)cn12]

or a pharmaceutically acceptable salt, hydrate, or solvate thereof.
Embodiment No. 146′ The method, conditioning regimen, population, or combination of any one of the preceding embodiments, wherein the aryl hydrocarbon receptor antagonist is compound (9)

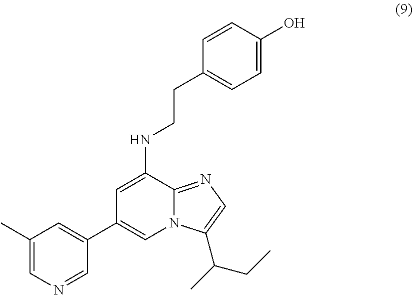 [SMILES: CCC(C)c1cnc2c(NCCc3ccc(O)cc3)cc(-c3cncc(C)c3)cn12]

or a pharmaceutically acceptable salt, hydrate, or solvate thereof.
Embodiment No. 147: The method, conditioning regimen, population, or combination of any one of the preceding embodiments, wherein the aryl hydrocarbon receptor antagonist is compound (10)

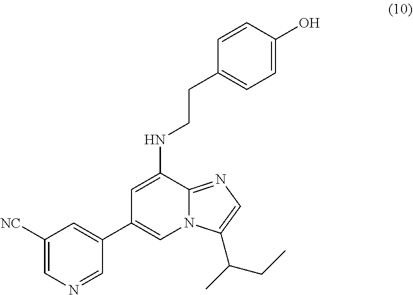 [SMILES: CCC(C)c1cnc2c(NCCc3ccc(O)cc3)cc(-c3cncc(C#N)c3)cn12]

or a pharmaceutically acceptable salt, hydrate, or solvate thereof.
Embodiment No. 148: The method, conditioning regimen, population, or combination of any one of the preceding embodiments, wherein the aryl hydrocarbon receptor antagonist is compound (11)

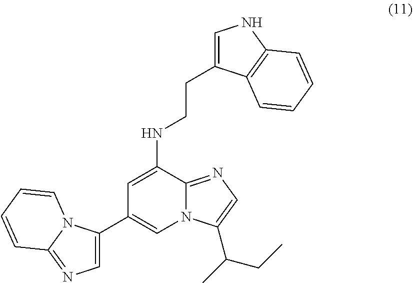 [SMILES: CCC(C)c1cnc2c(NCCc3c[nH]c4ccccc34)cc(-c3cnc4ccccn34)cn12]

or a pharmaceutically acceptable salt, hydrate, or solvate thereof.
Embodiment No. 149: The method, conditioning regimen, population, or combination of any one of the preceding embodiments, wherein the aryl hydrocarbon receptor antagonist is compound (12)

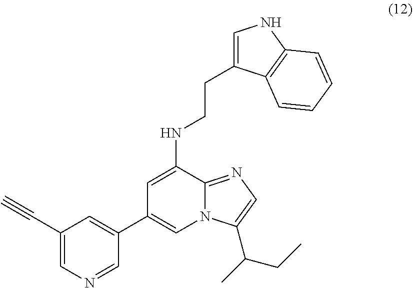 [SMILES: C#Cc1cncc(-c2cc(NCCc3c[nH]c4ccccc34)c3ncc(C(C)CC)n3c2)c1]

or a pharmaceutically acceptable salt, hydrate, or solvate thereof.
Embodiment No. 150: The method, conditioning regimen, population, or combination of any one of the preceding embodiments, wherein the aryl hydrocarbon receptor antagonist is compound (13)

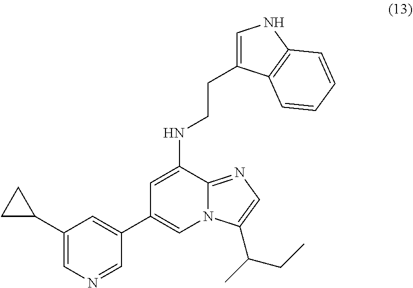 [SMILES: CCC(C)c1cnc2c(NCCc3c[nH]c4ccccc34)cc(-c3cncc(C4CC4)c3)cn12]

or a pharmaceutically acceptable salt, hydrate, or solvate thereof.
Embodiment No. 151: The method, conditioning regimen, population, or combination of any one of the preceding embodiments, wherein the aryl hydrocarbon receptor antagonist is compound (25)

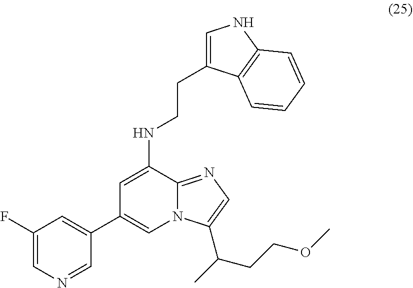 [SMILES: COCCC(C)c1cnc2c(NCCc3c[nH]c4ccccc34)cc(-c3cncc(F)c3)cn12]

or a pharmaceutically acceptable salt, hydrate, or solvate thereof.
Embodiment No. 152. The method, conditioning regimen, population, or combination of any one of the preceding embodiments, wherein the aryl hydrocarbon receptor antagonist is compound (27)

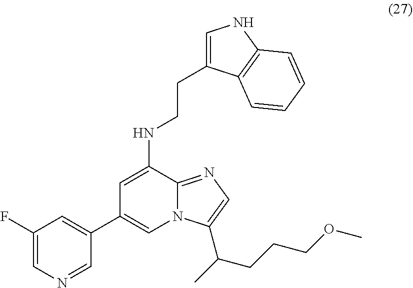 [SMILES: COCCCC(C)c1cnc2c(NCCc3c[nH]c4ccccc34)cc(-c3cncc(F)c3)cn12]

or a pharmaceutically acceptable salt, hydrate, or solvate thereof.
Embodiment No. 153: The method, conditioning regimen, population, or combination of any one of the preceding embodiments, wherein the aryl hydrocarbon receptor antagonist is compound (28)

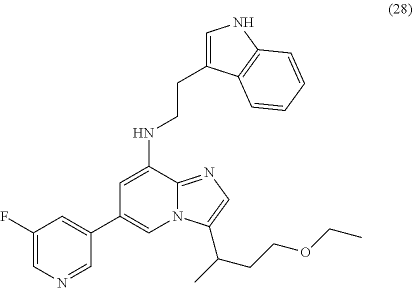 [SMILES: CCOCCC(C)c1cnc2c(NCCc3c[nH]c4ccccc34)cc(-c3cncc(F)c3)cn12]

or a pharmaceutically acceptable salt, hydrate, or solvate thereof.
Embodiment No. 154: The method, conditioning regimen, population, or combination of any one of the preceding embodiments, wherein the aryl hydrocarbon receptor antagonist is a compound represented by formula (V)

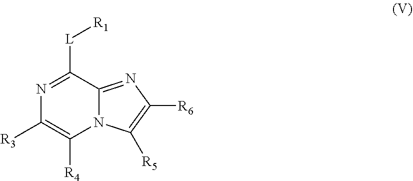

or a pharmaceutically acceptable salt, hydrate, or solvate thereof, wherein:

L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ and R_{8b} are each independently selected from the group consisting of hydrogen and optionally substituted C1-4 alkyl, and each n is independently an integer from 2 to 6;

R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂R_{9b}, —NR₉ₐC(O)OR_{9b}, —OC(O)NR₉ₐR_{9b}R_{9c}, —OC(S)NR₉ₐR_{9b}R_{9c}, optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl:

R₃ is selected from the group consisting of optionally substituted aryl, optionally substituted heteroaryl, optionally substituted cycloalkyl, and optionally substituted heterocycloalkyl:

R₄ is selected from the group consisting of hydrogen and optionally substituted C1-4 alkyl;

R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and

R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

Embodiment No. 155: The method, conditioning regimen, population, or combination of any one of the preceding embodiments, wherein the aryl hydrocarbon receptor antagonist is compound (14)

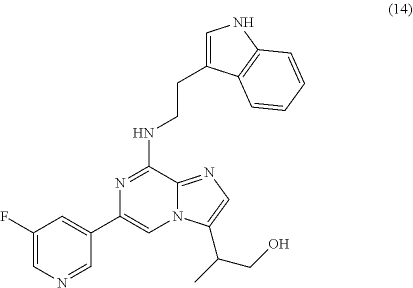 [SMILES: CC(CO)c1cnc2c(NCCc3c[nH]c4ccccc34)nc(-c3cncc(F)c3)cn12]

or a pharmaceutically acceptable salt, hydrate, or solvate thereof.
Embodiment No. 156: The method, conditioning regimen, population, or combination of any one of the preceding embodiments, wherein the aryl hydrocarbon receptor antagonist is compound (15)

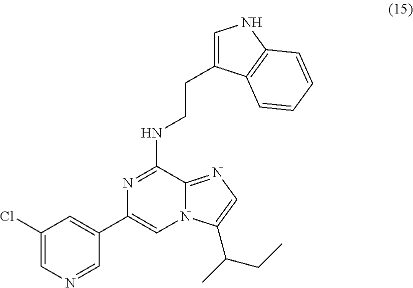 [SMILES: CCC(C)c1cnc2c(NCCc3c[nH]c4ccccc34)nc(-c3cncc(Cl)c3)cn12]

or a pharmaceutically acceptable salt, hydrate, or solvate thereof.
Embodiment No. 157: The method, conditioning regimen, population, or combination of any one of the preceding embodiments, wherein the aryl hydrocarbon receptor antagonist is compound (16)

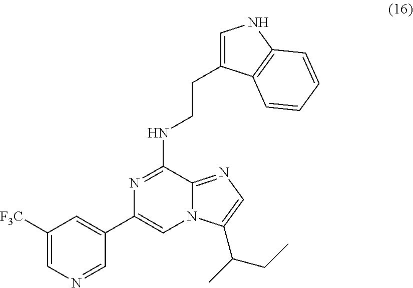 [SMILES: CCC(C)c1cnc2c(NCCc3c[nH]c4ccccc34)nc(-c3cncc(C(F)(F)F)c3)cn12]

or a pharmaceutically acceptable salt, hydrate, or solvate thereof.
Embodiment No. 158: The method, conditioning regimen, population, or combination of any one of the preceding embodiments, wherein the aryl hydrocarbon receptor antagonist is compound (17)

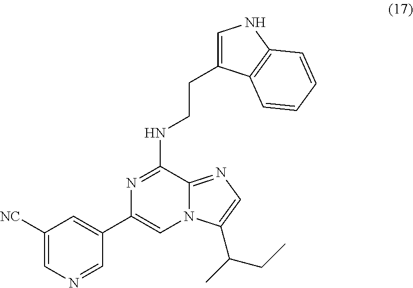 [SMILES: CCC(C)c1cnc2c(NCCc3c[nH]c4ccccc34)nc(-c3cncc(C#N)c3)cn12]

or a pharmaceutically acceptable salt, hydrate, or solvate thereof.
Embodiment No. 159: The method, conditioning regimen, population, or combination of any one of the preceding embodiments, wherein the aryl hydrocarbon receptor antagonist is compound (18)

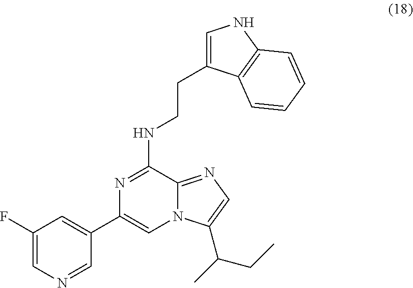 [SMILES: CCC(C)c1cnc2c(NCCc3c[nH]c4ccccc34)nc(-c3cncc(F)c3)cn12]

or a pharmaceutically acceptable salt, hydrate, or solvate thereof.
Embodiment No. 160: The method, conditioning regimen, population, or combination of any one of the preceding embodiments, wherein the aryl hydrocarbon receptor antagonist is compound (19)

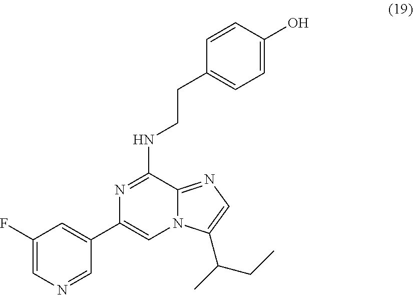 [SMILES: CCC(C)c1cnc2c(NCCc3ccc(O)cc3)nc(-c3cncc(F)c3)cn12]

or a pharmaceutically acceptable salt, hydrate, or solvate thereof.
Embodiment No. 161: The method, conditioning regimen, population, or combination of any one of the preceding embodiments, wherein the aryl hydrocarbon receptor antagonist is compound (20)

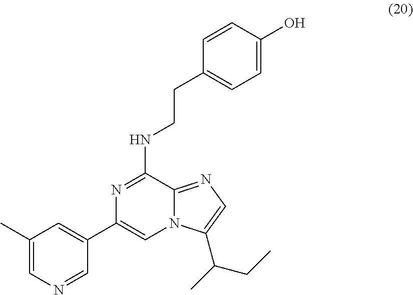 [SMILES: CCC(C)c1cnc2c(NCCc3ccc(O)cc3)nc(-c3cncc(C)c3)cn12]

or a pharmaceutically acceptable salt, hydrate, or solvate thereof.
Embodiment No. 162: The method, conditioning regimen, population, or combination of any one of the preceding embodiments, wherein the aryl hydrocarbon receptor antagonist is compound (21)

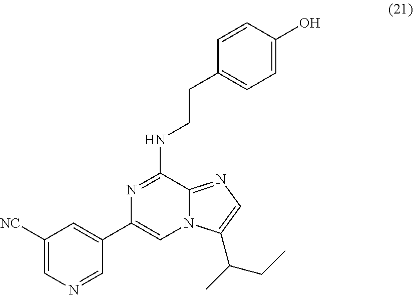 [SMILES: CCC(C)c1cnc2c(NCCc3ccc(O)cc3)nc(-c3cncc(C#N)c3)cn12]

or a pharmaceutically acceptable salt, hydrate, or solvate thereof.
Embodiment No. 163: The method, conditioning regimen, population, or combination of any one of the preceding embodiments, wherein the aryl hydrocarbon receptor antagonist is compound (22)

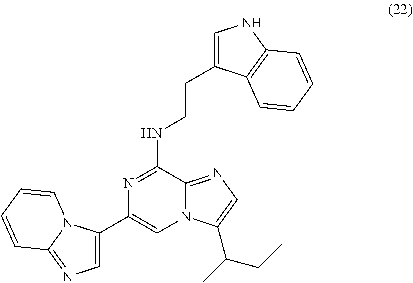 [SMILES: CCC(C)c1cnc2c(NCCc3c[nH]c4ccccc34)nc(-c3cnc4ccccn34)cn12]

or a pharmaceutically acceptable salt, hydrate, or solvate thereof.
Embodiment No. 164: The method, conditioning regimen, population, or combination of any one of the preceding embodiments, wherein the aryl hydrocarbon receptor antagonist is compound (23)

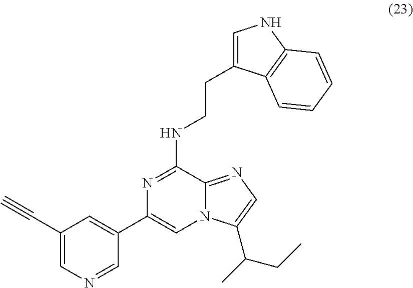 [SMILES: C#Cc1cncc(-c2cn3c(C(C)CC)cnc3c(NCCc3c[nH]c4ccccc34)n2)c1]

or a pharmaceutically acceptable salt, hydrate, or solvate thereof.
Embodiment No. 165: The method, conditioning regimen, population, or combination of any one of the preceding embodiments, wherein the aryl hydrocarbon receptor antagonist is compound (24)

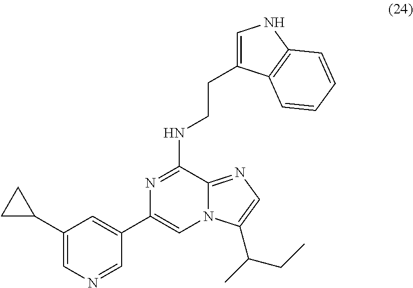 [SMILES: CCC(C)c1cnc2c(NCCc3c[nH]c4ccccc34)nc(-c3cncc(C4CC4)c3)cn12]

or a pharmaceutically acceptable salt, hydrate, or solvate thereof.
Embodiment No. 166: The method, conditioning regimen, population, or combination of any one of the preceding embodiments, wherein the aryl hydrocarbon receptor antagonist is compound (26)

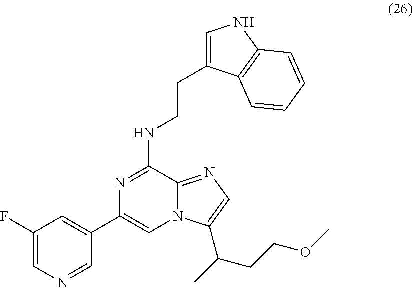 [SMILES: COCCC(C)c1cnc2c(NCCc3c[nH]c4ccccc34)nc(-c3cncc(F)c3)cn12]

or a pharmaceutically acceptable salt, hydrate, or solvate thereof.
Embodiment No. 167: The method, conditioning regimen, population, or combination of any one of the preceding embodiments, wherein the aryl hydrocarbon receptor antagonist is compound (29)

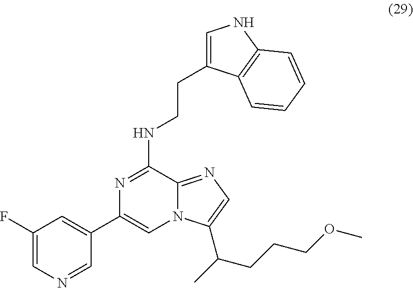 [SMILES: COCCCC(C)c1cnc2c(NCCc3c[nH]c4ccccc34)nc(-c3cncc(F)c3)cn12]

or a pharmaceutically acceptable salt, hydrate, or solvate thereof.
Embodiment No. 168: The method, conditioning regimen, population, or combination of any one of the preceding embodiments, wherein the aryl hydrocarbon receptor antagonist is compound (30)

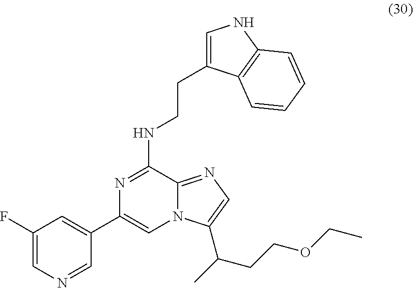 [SMILES: CCOCCC(C)c1cnc2c(NCCc3c[nH]c4ccccc34)nc(-c3cncc(F)c3)cn12]

or a pharmaceutically acceptable salt, hydrate, or solvate thereof.
Embodiment No. 169: The method, conditioning regimen, population, or combination of any one of the preceding embodiments, wherein the population, prior to expansion, comprises no more than 9×10⁷ CD34+ cells.
Embodiment No. 170: The method, conditioning regimen, population, or combination of any one of the preceding embodiments, wherein the population, prior to expansion, comprises no more than 8×10⁷ CD34+ cells.
Embodiment No. 171: The method, conditioning regimen, population, or combination of any one of the preceding embodiments, wherein the population, prior to expansion, comprises no more than 7×10⁷ CD34+ cells.
Embodiment No. 172: The method, conditioning regimen, population, or combination of any one of the preceding embodiments, wherein the population, prior to expansion, comprises no more than 6×10⁷ CD34+ cells.
Embodiment No. 173: The method, conditioning regimen, population, or combination of any one of the preceding embodiments, wherein the population, prior to expansion, comprises no more than 5×10⁷ CD34+ cells.
Embodiment No. 174: The method, conditioning regimen, population, or combination of any one of the preceding embodiments, wherein the population, prior to expansion, comprises no more than 9×10⁶ CD34+ cells.
Embodiment No. 175: The method, conditioning regimen, population, or combination of any one of the preceding embodiments, wherein the population, prior to expansion, comprises no more than 8×10⁶ CD34+ cells.
Embodiment No. 176: The method, conditioning regimen, population, or combination of any one of the preceding embodiments, wherein the population, prior to expansion, comprises no more than 7×10⁶ CD34+ cells.
Embodiment No. 177: The method, conditioning regimen, population, or combination of any one of the preceding embodiments, wherein the population, prior to expansion, comprises no more than 6×10⁶ CD34+ cells.
Embodiment No. 178: The method, conditioning regimen, population, or combination of any one of the preceding embodiments, wherein the population, prior to expansion, comprises no more than 5×10⁶ CD34+ cells.
Embodiment No. 179: The method, conditioning regimen, population, or combination of any one of the preceding embodiments, wherein the population, prior to expansion, comprises no more than 1×10⁸ CD34+ cells.
Embodiment No. 180: The method, conditioning regimen, population, or combination of any one of the preceding embodiments, wherein the expanding comprises contacting the CD34+ cells with an aryl hydrocarbon receptor antagonist, preferably wherein the aryl hydrocarbon receptor antagonist is SR-1, compound 2, a compound represented by formula (IV), or a compound represented by formula (V).
Embodiment No. 181: The method, conditioning regimen, population, or combination of any one of the preceding embodiments, wherein prior to infusion into the patient, the hematopoietic stem or progenitor cells are mobilized and isolated from a donor.
Embodiment No. 182: The method, conditioning regimen, population, or combination of any one of the preceding embodiments, wherein the donor is a human.
Embodiment No. 183: The method, conditioning regimen, population, or combination of any one of the preceding embodiments, wherein the hematopoietic stem or progenitor cells are mobilized by contacting the hematopoietic stem or progenitor cells with a mobilizing amount of a CXCR4 antagonist and/or a CXCR2 agonist.
Embodiment No. 184: The method, conditioning regimen, population, or combination of any one of the preceding embodiments, wherein the CXCR4 antagonist is plerixafor.
Embodiment No. 185: The method, conditioning regimen, population, or combination of any one of the preceding embodiments, wherein the CXCR4 antagonist is BL-8040.
Embodiment No. 186: The method, conditioning regimen, population, or combination of any one of the preceding embodiments, wherein the CXCR2 agonist is Gro-s, Gro-s T, or a variant thereof.
Embodiment No. 187: The method, conditioning regimen, population, or combination of any one of the preceding embodiments, wherein the patient is a human.
Embodiment No. 188: The method, conditioning regimen, population, or combination of any one of the preceding embodiments, wherein the stem cell disorder is a hemoglobinopathy disorder.
Embodiment No. 189: The method, conditioning regimen, population, or combination of any one of the preceding embodiments, wherein the hemoglobinopathy disorder is selected from the group consisting of sickle cell anemia, thalassemia, Fanconi anemia, aplastic anemia, and Wiskott-Aldrich syndrome.
Embodiment No. 190: The method, conditioning regimen, population, or combination of any one of the preceding embodiments, wherein the stem cell disorder is a myelodysplastic disorder.
Embodiment No. 191: The method, conditioning regimen, population, or combination of any one of the preceding embodiments, wherein the stem cell disorder is an immunodeficiency disorder.
Embodiment No. 192: The method, conditioning regimen, population, or combination of any one of the preceding embodiments, wherein the immunodeficiency disorder is a congenital immunodeficiency.
Embodiment No. 193: The method, conditioning regimen, population, or combination of any one of the preceding embodiments, wherein the immunodeficiency disorder is an acquired immunodeficiency.
Embodiment No. 194: The method, conditioning regimen, population, or combination of any one of the preceding embodiments, wherein the acquired immunodeficiency is human immunodeficiency virus or acquired immune deficiency syndrome.
Embodiment No. 195′ The method, conditioning regimen, population, or combination of any one of the preceding embodiments, wherein the stem cell disorder is a metabolic disorder.
Embodiment No. 196′ The method, conditioning regimen, population, or combination of any one of the preceding embodiments, wherein the metabolic disorder is selected from the group consisting of glycogen storage diseases, mucopolysaccharidoses, Gaucher disease, Hurler disease, sphingolipidoses, metachromatic leukodystrophy, globoid cell leukodystrophy, and cerebral adrenoleukodystrophy.
Embodiment No. 197: The method, conditioning regimen, population, or combination of any one of the preceding embodiments, wherein the metabolic disorder is an inherited metabolic disorder.
Embodiment No. 198: The method, conditioning regimen, population, or combination of any one of the preceding embodiments, wherein the inherited metabolic disorder is Hurler disease, metachromatic leukodystrophy, globoid cell leukodystrophy, or cerebral adrenoleukodystrophy.
Embodiment No. 199: The method, conditioning regimen, population, or combination of any one of the preceding embodiments, wherein the stem cell disorder is cancer.
Embodiment No. 200: The method, conditioning regimen, population, or combination of any one of the preceding embodiments, wherein the cancer is selected from the group consisting of leukemia, lymphoma, multiple myeloma, and neuroblastoma.
Embodiment No. 201: The method, conditioning regimen, population, or combination of any one of the preceding embodiments, wherein the cancer is a hematological cancer.
Embodiment No. 202: The method, conditioning regimen, population, or combination of any one of the preceding embodiments, wherein the cancer is acute myeloid leukemia.
Embodiment No. 203: The method, conditioning regimen, population, or combination of any one of the preceding embodiments, wherein the cancer is acute lymphoid leukemia.
Embodiment No. 204: The method, conditioning regimen, population, or combination of any one of the preceding embodiments, wherein the cancer is chronic myeloid leukemia.
Embodiment No. 205: The method, conditioning regimen, population, or combination of any one of the preceding embodiments, wherein the cancer is chronic lymphoid leukemia.
Embodiment No. 206: The method, conditioning regimen, population, or combination of any one of the preceding embodiments, wherein the cancer is multiple myeloma.
Embodiment No. 207: The method, conditioning regimen, population, or combination of any one of the preceding embodiments, wherein the cancer is diffuse large B-cell lymphoma.
Embodiment No. 208: The method, conditioning regimen, population, or combination of any one of the preceding embodiments, wherein the cancer is non-Hodgkin's lymphoma.
Embodiment No. 209: The method, conditioning regimen, population, or combination of any one of the preceding embodiments, wherein the stem cell disorder is a disorder selected from the group consisting of adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.
Embodiment No. 210: The method, conditioning regimen, population, or combination of any one of the preceding embodiments wherein the stem cell disorder is an autoimmune disorder.
Embodiment No. 211: The method, conditioning regimen, population, or combination of any one of the preceding embodiments, wherein the autoimmune disorder is selected from the group consisting of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis.
Embodiment No. 212: The method, conditioning regimen, population, or combination of any one of the preceding embodiments, wherein the hematopoietic stem or progenitor cells are autologous with respect to the patient.
Embodiment No. 213: The method, conditioning regimen, population, or combination of any one of the preceding embodiments, wherein the hematopoietic stem or progenitor cells are allogeneic with respect to the patient.
Embodiment No. 214: The method, conditioning regimen, population, or combination of any one of the preceding embodiments, wherein the hematopoietic stem or progenitor cells are HLA-matched with respect to the patient.
Embodiment No. 215: The method, conditioning regimen, population, or combination of any one of the preceding embodiments, wherein the method reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu). For example, in some embodiments, the method reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu), fludarabine (Flu), and ATG (e.g., rATG).
Embodiment No. 216′ The method, conditioning regimen, population, or combination of any one of the preceding embodiments, wherein the method reduces the risk of autoimmune cytopenia in the patient by about 1% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more, as compared to a comparable method in which the patient is conditioned prior to receiving the population of expanded hematopoietic stem or progenitor cells by a conditioning regimen comprising administering to the patient busulfan (Bu) and fludarabine (Flu).
Embodiment No. 217: The method, conditioning regimen, population, or combination of any one of the preceding embodiments, wherein the method prevents the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).
Embodiment No. 218: The method, conditioning regimen, population, or combination of any one of the preceding embodiments, wherein the method prevents autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).
Embodiment No. 219: The method, conditioning regimen, population, or combination of any one of the preceding embodiments, wherein the method reduces the severity of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).
Embodiment No. 220: The method, conditioning regimen, population, or combination of any one of the preceding embodiments, wherein the method reduces the severity of autoimmune cytopenia in the patient by about 1% or more, about 2% or more, about 3% or more, about 4% or more, about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more, as compared to a comparable method in which the patient is conditioned prior to receiving the population of expanded hematopoietic stem or progenitor cells by a conditioning regimen comprising administering to the patient busulfan (Bu) and fludarabine (Flu).
Embodiment No. 221: The method, conditioning regimen, population, or combination of any one of the preceding embodiments, wherein in the comparable method, the patient is conditioned prior to receiving the population of expanded hematopoietic stem or progenitor cells by a conditioning regimen comprising administering to the patient busulfan (Bu), fludarabine (Flu), and ATG (e.g., rATG).
Embodiment No. 222: The method, conditioning regimen, population, or combination of any one of the preceding embodiments, wherein in the comparable method, the patient is conditioned prior to receiving the population of expanded hematopoietic stem or progenitor cells by a conditioning regimen as described in Example 2.
Embodiment No. 223: The method, conditioning regimen, population, or combination of any one of the preceding embodiments, wherein the comparable method is substantially the same as the method other than that the patient is conditioned prior to receiving the population of expanded hematopoietic stem or progenitor cells by a conditioning regimen comprising administering to the patient busulfan (Bu) and fludarabine (Flu).
Embodiment No. 224: The method, conditioning regimen, population, or combination of any one of the preceding embodiments, wherein the comparable method is substantially the same as the method other than that the patient is conditioned prior to receiving the population of expanded hematopoietic stem or progenitor cells by a conditioning regimen comprising administering to the patient busulfan (Bu), fludarabine (Flu), and ATG (e.g., rATG).
Embodiment No. 225: A kit comprising a plurality of hematopoietic stem or progenitor cells and a package insert, wherein the package insert instructs a user to perform the method of any one of the preceding embodiments.

### EXAMPLES

### Example 1: Busulfan, Cyclophosphamide, ATG Conditioning Regimen

### Conditioning Regimen

The dosing scheme employs Busulfan (BU), Cyclophosphamide (CY) and rabbit Anti-thymocyte globulin (ATG) serotherapy (see, e.g., Bartelink et al (2008) BBMT 14:88-98, Bartelink et al (2009) BBMT 15:231-241, Bartelink et al (2014) BBMT 20:345-353 and Prasad et al (2008) Blood 112:2979-2989.)

The conditioning regimen is initiated on Day −9 (see Table 1 below) and consists of:

BU is administered via IV for 4 days (Days −9 to −6). BU dosing targets a cumulative AUC of 74-82 mg*hr/L with a goal of 78 mg*hr/L (equivalent to 18,050-20,000 μM*min/L with a goal of 19,025 μM*min/L).

(A) Patients receive a total of 4 doses (recommended initial dose: 120 mg/m2 for age >1 yr and 80 mg/m² for age <1 yr) given once daily over 3 hours and adjusted to achieve targeted dose range.

or

(B) Patients receive a total of 16 doses (recommended initial dose 1 mg/kg) given every 6 hours and adjusted to maintain a steady state concentration (Cₛₛ) of 770-850 ng/mL with a goal of 810 ng/mL.

Leveteracetam (Keppra) prophylaxis against seizures is given starting with a loading dose on the day before the first dose of BU and continued until at least 24 hours after the last dose of BU.

CY is administered via IV for 4 days (Days −5 to −2) at a dose of 50 mg/kg each day (total dose 200 mg/kg). Mesna prophylaxis is administered per institutional protocol. CY dose is adjusted if the patient's actual body weight is more than 125% of the ideal body weight. First dose of CY begins >24 hours after the last dose of BU.

ATG (rabbit: Thymoglobulin-Sanofi/Genzyme) is administered over at least 6 hours at 2.5 mg/kg/dose each day on days −5 to −2. If no adverse reactions are present, the infusion rate may be increased per institutional guidelines.

Graft-versus-host disease (GVHD) prophylaxis consists of cyclosporine A (CsA) and Mycophenolate Mofetil (MMF). Pre-medications for the conditioning regimens are administered per institutional guidelines.

TABLE 1
Conditioning Regimen Scheme and GVHD Prophylaxis

| | | GVHD |
|---|---|---|
| Day | Conditioning Regimen | Prophylaxis |
| Day −9 | BU^{a} IV given once daily over 3 hours (A) OR | — |
| | q6h IV given over 2 hours (B) | |
| Day −8 | BU^{a} IV given once daily over 3 hours (A) OR | — |
| | q6h IV given over 2 hours (B) | |
| Day −7 | BU^{a} IV given once daily over 3 hours (A) OR | — |
| | q6h IV given over 2 hours (B) | |
| Day −6 | BU^{a} IV given once daily over 3 hours (A) OR | — |
| | q6h IV given over 2 hours (B) | |
| Day −5 | CY 50 mg/kg once daily over 1 hour + Mesna | — |
| | prophylaxis ATG once daily IV over at least | |
| | 6 hours^{b} | |
| Day −4 | CY 50 mg/kg once daily over 1 hour + Mesna | — |
| | prophylaxis ATG once daily IV over at least | |
| | 6 hours^{b} | |
| Day −3 | CY 50 mg/kg once daily over 1 hour + Mesna | Begin CsA |
| | prophylaxis ATG once daily IV over at least | and MMF |
| | 6 hours^{b} | |
| Day −2 | CY 50 mg/kg once daily over 1 hour + Mesna | — |
| | prophylaxis ATG once daily IV over at least | |
| | 6 hours^{b} | |
| Day −1 | | — |
| Day 0 | MGTA-456 infusion | |
| Day 1 | — | Begin |
| | | G-CSF^{c} |

Abbreviations:
CY = cyclophosphamide;
BU = busulfan;
ATG = antithrombocyte globulin (Thymoglobulin);
CsA—Cyclosporine;
MMF = mycophenolate mofetil;
G-CSF = granulocyte-colony stimulating factor
^{a}BU targeted to achive (A) cumulative AUC 74-82 mg-h/L (18,050-20,000 μM-min/L) or (B) 770-850 ng/mL Cₛₛ.
^{b}If the first dose of ATG is well-tolerated, subsequent doses may be administered over a shorter time frame per institutional guidelines.
^{c}G-CSF started on day +1 and continued until the ANC ≥2,500/μL for 2 consecutive days.

### Dosing of Busulfan

Busulfan dosing targets a cumulative AUC of 74-82 mg*hr/L with a goal of 78 mg*hr/L

(equivalent to 18,050-20,000 μM*min/L with a goal of 19,025 μM*min/L).

(A) Once daily dosing: Busulfan is given by IV over 3 hours and adjusted to maintain an AUC target of 74-82 mg-h/L (4400-4800 μM-min/day). The recommended calculation of the AUC is performed on blood samples (obtained pre-dose, 15 minutes, 1 hr, 3 hr, 5 hr and 7 hr after the end of infusion). Busulfan dose is adjusted when the predicted cumulative AUC falls outside the range. If possible, an evaluation of the AUC after dose adjustment may be performed on subsequent days and used to confirm total busulfan exposure is within the range. Busulfan PK may alternatively be calculated based on institutional guidelines, including the use of test doses, to achieve the specific target AUC. Initial starting busulfan doses may also be calculated according to institutional guidelines.

or

(B) Q6h dosing: Busulfan is given by IV over 2 hours and adjusted to achieve a steady state 770-850 ng/mL with a goal of 810 ng/mL. Pharmacokinetics (PK) will be performed per institutional protocol to maintain target steady state concentration. Recommended PK sampling times are: Pre-dose, 60 min, 115 min (5 min before end), 150 min, 3 hours, 4 hours, 5 hours, 6 hours (prior to next dose). Busulfan PK and initial starting dose may alternatively be calculated based on institutional guidelines to achieve the specified target steady state concentration.

Calculations used for AUC/Cₛₛ conversions are as follows:

AUC (μM*min)/L*246 μg/μM*1 L/1000 mL=AUG (mg*min)/mL

AUC (μM*min)/L*246 μg/μM*1 mg/1000 μg*1 hr/60 min=AUC (mg*h)/L

AUC (μM*min)/L*246 μg/μM*1 hr/60 min*1000 ng/1 μg*1111000 mL/(Dosing frequency)=Css(ng/mL)

### Dosing of Cyclophosphamide

Route of Administration: The total daily dose is given as a 1-hour IV infusion in D5NS (Dextrose 5% in Normal Saline (0.9%)). Patients should receive additional hydration with 3000 mL/m²/day of appropriate maintenance IV fluids starting 10 hours prior to CY and continued until 24 hours after the last dose. Patients will also receive mesna prophylaxis per institutional guidelines.

Dose Adjustment: Dose adjustment is required if the actual body weight (ABW) is more than 125% of the ideal body weight (IBW) for the age and gender. The dose should be calculated using adjusted ideal body weight (AIBW) calculations as follows:

Less than 60 inches:

IBW=(ht2×1.65)/1000 where ht=cm, IBW=kg

More than 60 inches:

Males IBW=39.0+[2.27×(ht−60)] where ht=inches, IBW=kg

Females IBW=42.2+[2.27×(ht−60)] where ht=inches, IBW=kg

Adjusted ideal body weight formula:

AIBW=IBW+[(0.25)×(ABW−IBW)

### Immunosuppression

All subjects receive the immunosuppression regimen per the study treatment plan as outlined in the below:

Cyclosponne A (CsA): Initiate at Day −3. Administer per institutional guidelines with adjustment to maintain target serum trough levels of 200-400 ng/mL. CsA weaning begins at Day 270 if the patient is stably engrafted and has no active GVHD. The dose is tapered to zero by 10% weekly dose reduction over approximately 10 weeks.

Mycophenolate mofetil (MMF): Initiate on Day −3 and continue until Day +45 or 7 days after engraftment whichever is later if no acute GVHD is seen. Weaning per institutional guidelines. MMF can be administered IV or PO. For patients <50 kg 15 mg/kg IV/PO TID and for patients over 50 kg 1 g IV/PO TID.

### Example 2: Busulfan, Fludarabine, ATG Conditioning Regimen

### Conditioning Regimen

The dosing scheme employed Busulfan (BU), Fludarabine (FLU) and rabbit Anti-thymocyte globulin (ATG).

The conditioning regimen is initiated on Day −9 (see Table 2 below) and consists of:

ATG (rabbit; Thymoglobulin) is administered over 6 hours beginning on day −9 per nomogram based upon body weight and absolute lymphocyte count (ALC) (see Table 3 below).

FLU is administered at 40 mg/m²/day IV (over 1 hour at a constant rate) for 4 days (Day −5 to −2) for a total dose of 160 mg/m². Note: For children weighing <10 kg, FLU dosing is 1.33 mg/kg IV (over 1 hour at a constant rate) for 4 days (Day −5 to −2) for a total dose of 5.33 mg/kg.

BU is administered via IV for 4 days (Days −5 to −2) after FLU. Patients receive 4 doses of BU targeting total BU exposure of 21,000 to 22,000 μM/min/L⁻¹.

GVHD prophylaxis consists of cyclosporine A (CsA) and methylprednisolone (MP) and should be administered as follows. CsA is given daily starting from Day −2 until Day 180 beginning with a dose of 2.5 mg/kg IV every 12 hours for children 245 kg and every 8 hours for children <45 kg. CsA blood trough levels are monitored and maintained per standard practice (the target CsA trough level is 200 mg/L to 400 mg/L). CsA taper begins at Day 180 if the patient is stably engrafted and has no active GVHD. The dose is tapered to zero by 10% weekly dose reduction over approximately 10 weeks.

MP is administered starting from Day 0 [(0.5 mg/kg intravenously (IV) twice daily (1.0 mg/kg/day)] through Day 28. At that time, the dose is tapered to 0.25 mg/kg IV twice daily (0.5 mg/kg/day) on Day 29 through Day 35 and subsequently to 0.25 mg/kg IV once daily (0.25 mg/kg/day) on Day 36 through Day 42. Following neutrophil engraftment (first of 3 consecutive days with ANC >0.5×10⁹ neutrophils/L), the patient may be switched to oral prednisolone/prednisone using the following conversion: (1.25×IV MP dose).

Pre-medications for the conditioning regimen should be administered as follows. Levetiracetam is administered to all patients in accordance with institutional guidelines as seizure prophylaxis during BU therapy. The following pre-medications are administered 30 minutes prior to each dose of ATG including: acetaminophen (10 mg/kg orally; maximum dose 500 mg); diphenhydramine (1 mg/kg IV or PO; maximum dose 50 mg), and methylprednisolone (1 mg/kg IV; maximum dose 125 mg). Ondansetron is administered as a continuous infusion per the institutional guidelines beginning prior to the first infusion of FLU and continuing through at least Day −1.

Pre-medication for the MGTA-456 infusion is per institution's standard of care for HSC product infusions.

TABLE 2
Conditioning Regimen Scheme and GVHD Prophylaxis

| | | GVHD |
|---|---|---|
| Day | Conditioning Regimen | Prophylaxis |
| Day −9 | ATG once daily IV over 6 hours per nomogram^{d,e} | — |
| Day −8 | ATG once daily IV over 6 hours per nomogram^{d,e} | — |
| Day −7 | ATG once daily IV over 6 hours per nomogram^{d,e} | — |
| Day −6 | ATG once daily IV over 6 hours per nomogram^{d,e} | — |
| Day −5 | FLU^{a} 40 mg/m² IV over 1 hour | — |
| | BU^{b} once daily IV over 3 hours | |
| Day −4 | FLU^{a} 40 mg/m² IV over 1 hour | — |
| | BU^{b} once daily IV over 3 hours | |
| Day −3 | FLU^{a} 40 mg/m² IV over 1 hour | — |
| | BU^{b} once daily IV over 3 hours | |
| Day −2 | FLU^{a} 40 mg/m² IV over 1 hour | Begin CsA |
| | BU^{b} once daily IV over 3 hours | |
| Day −1 | Rest | — |
| Day 0 | MGTA-456 infusion | Begin MP |
| Day 1 | — | Begin |
| | | G-CSF^{c} |

Abbreviations:
FLU = fludarabine;
BU = busulfan;
ATG = antithymocyte globulin (thymoglobulin);
CsA = Cyclosporine;
MP = methylprednisolone;
G-CSF = granulocyte-colony stimulating factor
^{a}FLU dosing for children <10 kg will be weight-based, using a dose of 1.33 mg/kg over 1 hour daily (therefore, total dose is 5.33 mg/kg).
^{b}BU will be targeted to achieve a total regimen exposure of 21,000 to 22,000 μM/min/L⁻¹.
^{c}G-CSF will be started when the ANC is ≤500/μL and continued until the ANC ≥2,500/μL for 2 consecutive days.
^{d}See Table 3.
^{e}If the first dose of ATG is well-tolerated, subsequent doses may be administered over a shorter time frame per institutional guidelines.

### Dosing of Busulfan

This protocol uses once daily (every 24 hour) intravenous BU dosing for conditioning.

Four (4) total doses of BU are given over 4 days in the preparative regimen (Days −5, −4, −3 and −2). AUC determination after each of the first 3 doses will be performed (AUC₁, AUC₂, and AUC₃ following Doses 01, #2 and #3). For these patients, the total regimen BU AUC targeted is 21,000-22,000 μM·min-L⁻¹ to optimize engraftment while minimizing toxicity. Each BU dose is administered over 3 hours by central venous line per Institutional standard guidelines on each day of administration. Each BU administration begins after completion of the daily FLU administration.

The initial empiric BU dosing (Dose #1) for all patients is determined as follows:

For patients weighing <12.5 kg: initial dose is based on the formula per the population PK model developed by Long-Boyle.

For patients weighing ≥12.5 kg and <66 kg: initial dose is determined by the nomogram adapted from Bartelink.

For patients weighing ≥66 kg: initial BU dose=3 mg/kg IV.

TABLE 3
ATG Dosing

| Patient | Absolute | Cumulative | Number | Daily |
|---|---|---|---|---|
| Weight | Lymphocyte | dose | of | dose |
| (kg)^{a} | Count^{b} | (mg/kg)^{c} | doses | (mg/kg) |
| 5 | 0.1 | 10 | 4 | 2.5 |
| 5 | 0.5 | 10 | 4 | 2.5 |
| 5 | 1 | 10 | 4 | 2.5 |
| 5 | 2 | 10 | 4 | 2.5 |
| 5 | 3 | 10 | 4 | 2.5 |
| 5 | 4 | 10 | 4 | 2.5 |
| 7.5 | 0.1 | 8 | 4 | 2.0 |
| 7.5 | 0.5 | 9 | 4 | 2.3 |
| 7.5 | 1 | 10 | 4 | 2.5 |
| 7.5 | 2 | 10 | 4 | 2.5 |
| 7.5 | 3 | 10 | 4 | 2.5 |
| 7.5 | 4 | 10 | 4 | 2.5 |
| 10 | 0.1 | 7 | 4 | 1.8 |
| 10 | 0.5 | 8 | 4 | 2.0 |
| 10 | 1 | 9 | 4 | 2.3 |
| 10 | 2 | 10 | 4 | 2.5 |
| 10 | 3 | 10 | 4 | 2.5 |
| 10 | 4 | 10 | 4 | 2.5 |
| 15 | 0.1 | 6 | 3 | 2.0 |
| 15 | 0.5 | 6 | 4 | 1.5 |
| 15 | 1 | 7 | 4 | 1.8 |
| 15 | 2 | 8 | 4 | 2.0 |
| 15 | 3 | 10 | 4 | 2.5 |
| 15 | 4 | 10 | 4 | 2.5 |
| 20 | 0.1 | 5 | 2 | 2.5 |
| 20 | 0.5 | 5 | 3 | 1.7 |
| 20 | 1 | 6 | 3 | 2.0 |
| 20 | 2 | 8 | 4 | 2.0 |
| 20 | 3 | 10 | 4 | 2.5 |
| 20 | 4 | 10 | 4 | 2.5 |
| 25 | 0.1 | 4 | 2 | 2.0 |
| 25 | 0.5 | 5 | 2 | 2.5 |
| 25 | 1 | 5 | 3 | 1.7 |
| 25 | 2 | 6 | 3 | 2.0 |
| 25 | 3 | 9 | 4 | 2.3 |
| 25 | 4 | 10 | 4 | 2.5 |
| 30 | 0.1 | 4 | 2 | 2.0 |
| 30 | 0.5 | 4.5 | 2 | 2.3 |
| 30 | 1 | 5 | 2 | 2.5 |
| 30 | 2 | 6 | 3 | 2.0 |
| 30 | 3 | 8 | 4 | 2.0 |
| 30 | 4 | 10 | 4 | 2.5 |
| 35 | 0.1 | 3.5 | 2 | 1.8 |
| 35 | 0.5 | 4 | 2 | 2.0 |
| 35 | 1 | 5 | 2 | 2.5 |
| 35 | 2 | 6 | 3 | 2.0 |
| 35 | 3 | 7.5 | 3 | 2.5 |
| 35 | 4 | 10 | 4 | 2.5 |
| 40 | 0.1 | 3 | 2 | 1.5 |
| 40 | 0.5 | 3.5 | 2 | 1.8 |
| 40 | 1 | 4 | 2 | 2.0 |
| 40 | 2 | 5 | 2 | 2.5 |
| 40 | 3 | 6 | 3 | 2.0 |
| 40 | 4 | 10 | 4 | 2.5 |
| 50 | 0.1 | 3 | 2 | 1.5 |
| 50 | 0.5 | 3 | 2 | 1.5 |
| 50 | 1 | 4 | 2 | 2.0 |
| 50 | 2 | 5 | 2 | 2.5 |
| 50 | 3 | 6 | 3 | 2.0 |
| 50 | 4 | 8 | 4 | 2.0 |
| 60 | 0.1 | 2.5 | 2 | 1.3 |
| 60 | 0.5 | 3 | 2 | 1.5 |
| 60 | 1 | 3 | 2 | 1.5 |
| 60 | 2 | 4 | 2 | 2.0 |
| 60 | 3 | 5 | 2 | 2.5 |
| 60 | 4 | 7.5 | 3 | 2.5 |
| 70 | 0.1 | 2.5 | 1 | 2.5 |
| 70 | 0.5 | 2.5 | 2 | 1.3 |
| 70 | 1 | 3 | 2 | 1.5 |
| 70 | 2 | 4 | 2 | 2.0 |
| 70 | 3 | 5 | 2 | 2.5 |
| 70 | 4 | 6 | 3 | 2.0 |
| 80 | 0.1 | 2 | 1 | 2.0 |
| 80 | 0.5 | 2.5 | 1 | 2.5 |
| 80 | 1 | 3 | 2 | 1.5 |
| 80 | 2 | 4 | 2 | 2.0 |
| 80 | 3 | 5 | 2 | 2.5 |
| 80 | 4 | 6 | 3 | 2.0 |

^{a}Recipient's weight in kilograms
^{b}Absolute lymphocyte count (×10³/μL) determined from the CBC diff obtained prior to first ATG dose on Day −10 or −9
^{c}Cumulative dose is an estimate; actual dose is determined by last 2 columns, number of doses × daily dose.

ATG dosing (daily dose and number of days) is determined according to Table 3 above. For instance, for a 10-kg child with a pre-conditioning ALC (measured Day −10 or −9 prior to first ATG dose) of 1.000/uL, ATG doing will be 2.3 mg/kg/day for 4 days (Days −9, −8, −7, and −8). For a 20-kg child with a pre-conditioning absolute lymphocyte count (ALC) (measured Day −10 or −9 prior to first ATG dose) of 500/uL, ATG dosing will be 1.7 mg/kg/day for 3 days (Days −9, −8, and −7).

All patients receive the first ATG dose on Day −9, regardless of the number of ATG doses they will receive.

For patients whose weights and ALC do not hit exact values shown in Table 3, then round to the closest value that is shown in Table 3. If the patient's weight or ALC falls at the midpoint between 2 values shown in Table 3, round up to the nearest value.

### EQUIVALENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of administering expanded hematopoietic stem or progenitor cell transplant therapy to a patient in need thereof, the method comprising:
infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);
wherein the method prevents or reduces the risk of autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

2. A method of administering expanded hematopoietic stem or progenitor cell transplant therapy to a patient in need thereof, the method comprising:
infusing into the patient a population of expanded hematopoietic stem or progenitor cells, wherein the patient was conditioned prior to receiving the population of expanded hematopoietic stem or progenitor cells by a conditioning regimen comprising administering to the patient busulfan (Bu), cyclophosphamide (Cy), and anti-thymocyte globulin (rabbit) (rATG);
wherein the method prevents, or reduces the severity of, autoimmune cytopenia in the patient as compared to a comparable method in which the patient is conditioned prior to receiving the population of expanded hematopoietic stem or progenitor cells by a conditioning regimen comprising administering to the patient busulfan (Bu) and fludarabine (Flu).

3. A method of preventing, or reducing the risk of, autoimmune cytopenia in a patient in need thereof, the method comprising:
i) conditioning the patient with a conditioning regimen; and
ii) administering to the patient a population of hematopoietic stem or progenitor cells.

4. A method of preventing, or reducing the risk of, autoimmune cytopenia in a patient in need thereof, the method comprising:
i) conditioning the patient with a conditioning regimen; and
ii) transplanting the patient with expanded cord blood: wherein the conditioning regimen does not comprise busulfan plus fludarabine (BuFlu).

5. A method comprising administering to the patient a population of hematopoietic stem or progenitor cells, wherein the patient has previously been conditioned with a conditioning regimen.

6. A method of administering hematopoietic stem cell transplantation therapy to a patient in need thereof, wherein the patient has previously been conditioned with a conditioning regimen, the method comprising infusing into the patient a population of hematopoietic stem or progenitor cells.

7. A method comprising:
a) conditioning the patient with a conditioning regimen; and
b) administering to (e.g., infusing into) the patient a population of hematopoietic stem or progenitor cells.

8. A method of preventing or reducing the risk of autoimmune cytopenia in a patient in need thereof, the method comprising:
administering a prophylactic agent prior to, during, or following transplant with expanded cord blood; and
transplanting the patient with expanded cord blood;
wherein the prophylactic agent inhibits the production of antibodies in the patient.

9. A method of preventing or reducing the risk of autoimmune cytopenia in a patient in need thereof, the method comprising:
conditioning the patient with a conditioning regimen; and
transplanting the patient with expanded cord blood;
wherein the conditioning regimen is not busulfan plus fludarabine (BuFlu).

10. A method of administering hematopoietic stem or progenitor cell transplant therapy to a patient in need thereof, the method comprising:
(a) conditioning the patient with a conditioning regimen; and
(b) infusing into the patient a population of hematopoietic stem or progenitor cells.

11. A method of preparing a patient for hematopoietic stem or progenitor cell transplantation, the method comprising conditioning the patient with a conditioning regimen.

12. A method of administering hematopoietic stem cell transplantation therapy to a patient in need thereof, wherein the patient has previously been conditioned with a conditioning regimen, the method comprising infusing into the patient a population of hematopoietic stem or progenitor cells.

13. A method of administering hematopoietic stem or progenitor cell transplant therapy to a patient in need thereof, the method comprising:
(a) conditioning the patient with a conditioning regimen, wherein the conditioning regimen comprises
(a1) administering busulfan (Bu);
(a2) administering cyclophosphamide (Cy); and
(a3) administering anti-thymocyte globulin (rabbit)(ATG); and
(b) infusing into the patient a population of hematopoietic stem or progenitor cells.

14. A method of administering hematopoietic stem or progenitor cell transplant therapy to a patient in need thereof, the method comprising:
(a) conditioning the patient with a conditioning regimen, wherein the conditioning regimen comprising
(a1) administering busulfan (Bu) prior to administering cyclophosphamide and prior to administering anti-thymocyte globulin (rabbit) (ATG);
(a2) administering cyclophosphamide (Cy) after administering busulfan (Bu) and simultaneously with administering anti-thymocyte globulin (rabbit) (ATG); and
(a3) administering anti-thymocyte globulin (rabbit) (ATG) after administering busulfan (Bu) and simultaneously with administering cyclophosphamide (Cy); and
(b) infusing into the patient a population of hematopoietic stem or progenitor cells.

15. A method of administering hematopoietic stem or progenitor cell transplant therapy to a patient in need thereof, the method comprising:
(a) conditioning the patient with a conditioning regimen, wherein the conditioning regimen comprising
(a1) administering busulfan (Bu) at days −9 to −6 prior to infusing into the patient a population of hematopoietic stem or progenitor cells;
(a2) administering cyclophosphamide (Cy) at days −5 to −2 prior to infusing into the patient a population of hematopoietic stem or progenitor cells; and
(a3) administering anti-thymocyte globulin (rabbit)(ATG) at days −5 to −2 prior to infusing into the patient a population of hematopoietic stem or progenitor cells; and
(b) infusing into the patient a population of hematopoietic stem or progenitor cells at day 0.

16. A method of administering hematopoietic stem or progenitor cell transplant therapy to a patient in need thereof, the method comprising:
(a) conditioning the patient with a conditioning regimen, wherein the conditioning regimen comprising
(a1) administering busulfan (Bu) at a dose wherein the plasma exposure as measured by cumulative AUC is maintained within a range of 74-82 mg*hr/L for 4 consecutive days at days −9 to −8 prior to infusing into the patient a population of hematopoietic stem or progenitor cells;
(a2) administering cyclophosphamide (Cy) at a dose of 50 mg/kg/day for 4 consecutive days at days −5 to −2 prior to infusing into the patient a population of hematopoietic stem or progenitor cells; and
(a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and
(b) infusing into the patient a population of hematopoietic stem or progenitor cells at day 0.

17. A method of administering hematopoietic stem or progenitor cell transplant therapy to a patient in need thereof, the method comprising:
(a) conditioning the patient with a conditioning regimen, wherein the conditioning regimen comprising
(a1) administering busulfan (Bu) at a dose wherein the plasma exposure as measured by steady state concentration (Css) is maintained within a range of 770-850 ng/mL for 4 consecutive days at days −9 to −6 prior to infusing into the patient a population of hematopoietic stem or progenitor cells;
(a2) administering cyclophosphamide (Cy) at a dose of 50 mg/kg/day for 4 consecutive days at days −5 to −2 prior to infusing into the patient a population of hematopoietic stem or progenitor cells; and
(a3) administering anti-thymocyte globulin (rabbit)(ATG) at a dose of 2.5 mg/kg/day for 4 consecutive days at days −5 to −2 prior to infusing into the patient a population of hematopoietic stem or progenitor cells; and
(b) infusing into the patient a population of hematopoietic stem or progenitor cells at day 0.

18. A method of administering hematopoietic stem or progenitor cell transplant therapy to a patient in need thereof in accordance with the method of any one of the preceding claims, the method comprising:
(a) expanding, ex vivo, a population of CD34+ cells comprising no more than 1×10⁸ CD34+ cells; and
(b) infusing into the patient the hematopoietic stem or progenitor cells, or progeny thereof, expanded in (a).

19. A method of administering hematopoietic stem or progenitor cell transplant therapy to a patient in need thereof in accordance with the method of any one of the preceding claims, the method comprising infusing into the patient a population of hematopoietic stem or progenitor cells that have been expanded ex vivo, wherein the population, prior to expansion, comprises no more than 1×10⁸ CD34+ cells.

20. A method of treating a stem cell disorder in a patient, the method comprising administering hematopoietic stem or progenitor cell transplant therapy to the patient in accordance with the method of any one of the preceding claims.

21. The method of any one of the preceding claims, wherein the expanded cord blood has been expanded with an aryl hydrocarbon receptor antagonist.

22. The method of any one of the preceding claims, wherein the conditioning regimen comprises a prophylactic agent.

23. The method of any one of the preceding claims, wherein the prophylactic agent is an anti-CD20 antibody.

24. The method of any one of the preceding claims, wherein the anti-CD20 antibody is rituximab.

25. The method of any one of the preceding claims, wherein the prophylactic agent is administered in combination with intravenous immunoglobulin (IVIG).

26. The method of any one of the preceding claims, wherein the conditioning regimen is busulfan plus cyclophosphamide (BuCy).

27. The method of any one of the preceding claims, wherein the conditioning regimen substantially ablates the patient's B-cells.

28. The method of any one of the preceding claims, wherein the expanded cord blood is MGTA-456.

29. The method of any one of the preceding claims, wherein the patient is 2 years old or younger.

30. The method of any one of the preceding claims, wherein the patient has an inherited metabolic disorder.

31. The method of any one of the preceding claims, wherein the inherited metabolic disorder is Hurler disease, metachromatic leukodystrophy, globoid cell leukodystrophy, or cerebral adrenoleukodystrophy.

32. The method of any one of the preceding claims, wherein the conditioning regimen comprises administering radiation and/or a prophylactic agent to the patient.

33. The method of any one of the preceding claims, wherein the conditioning regimen is administered prior to the infusing of a population of hematopoietic stem or progenitor cells.

34. The method of any one of the preceding claims, wherein the conditioning regimen comprises administering one or more agents selected from the group consisting of an alkylating agent and an anti-leukocyte globulin.

35. The method of any one of the preceding claims, wherein the conditioning regimen comprises administering at least one alkylating agent and at least one anti-leukocyte globulin simultaneously, sequentially, or in alteration.

36. The method of any one of the preceding claims, wherein the conditioning regimen comprises administering a first alkylating agent, a second alkylating agent, and an anti-leukocyte globulin simultaneously, sequentially, or in alteration.

37. The method of any one of the preceding claims, wherein the anti-thymocyte globulin is anti-thymocyte globulin (rabbit) (Thymoglobulin, ATG) and/or anti-thymocyte (equine) (Atgam).

38. The method of any one of the preceding claims, wherein the alkylating agent is busulfan (Bu) and/or cyclophosphamide (Cy).

39. The method of any one of the preceding claims, wherein upon transplantation, the risk of autoimmune cytopenia is prevented or reduced in the patient relative to a patient that is administered a conditioning regimen comprising busulfan and fludarabine (BuFlu) prior to transplantation.

40. The method of any one of the preceding claims, wherein the hematopoietic stem or progenitor cells, or progeny thereof, maintain hematopoietic stem cell functional potential after 2 or more days following infusion of the hematopoietic stem or progenitor cells into the patient.

41. The method of any one of the preceding claims, wherein the hematopoietic stem or progenitor cells, or progeny thereof, localize to hematopoietic tissue and/or reestablish hematopoiesis following infusion of the hematopoietic stem or progenitor cells into the patient.

42. The method of any one of the preceding claims, wherein upon infusion into the patient, the hematopoietic stem or progenitor cells give rise to recovery of a population of cells selected from the group consisting of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

43. The method of any one of the preceding claims, wherein the conditioning regimen does not comprise busulfan plus fludarabine (BuFlu).

44. The method of any one of the preceding claims, wherein the patient is 2 years old or younger.

45. The method of any one of the preceding claims, further comprising administering a prophylactic agent against seizures prior to, during, or following the administering of busulfan (Bu).

46. The method of any one of the preceding claims, wherein the prophylactic agent against seizures is levetiracetam (Keppra).

47. The method of any one of the preceding claims, further comprising administering a chemotherapy adjuvant prior to, during, or following the administering of cyclophosphamide.

48. The method of any one of the preceding claims, wherein the chemotherapy adjuvant is mesna (Mesnex).

49. The method of any one of the preceding claims, wherein the chemotherapy adjuvant is administered during the administering of cyclophosphamide.

50. The method of any one of the preceding claims, further comprising administering an immunosuppression regimen to the patient.

51. The method of any one of the preceding claims, wherein the immunosuppression regimen comprises administering at least one immunosuppressant agent.

52. The method of any one of the preceding claims, wherein the immunosuppressant agent is mycophenolate mofetil (MMF, CellCept), cyclosporine A (CsA), and/or salts or prodrugs thereof.

53. The method of any one of the preceding claims, wherein the method further comprises administering granulocyte colony-stimulating factor (G-CSF) prior to, during, or following the infusion of a population of hematopoietic stem or progenitor cells.

54. The method of any one of the preceding claims, wherein the hematopoietic stem or progenitor cells are expanded ex vivo prior to infusion into the patient.

55. The method of any one of the preceding claims, wherein the hematopoietic stem or progenitor cells are expanded ex vivo prior to infusion into the patient by contacting the hematopoietic stem or progenitor cells with at least one agent selected from the group consisting of an aryl hydrocarbon receptor antagonist, nicotinamide, UM729, and UM171.

56. The method of any one of the preceding claims, wherein the expanded cord blood or population of hematopoietic stem or progenitor cells is selected from the group consisting of MGTA-456, omidubicel (NiCord), and ECT-001.

57. The method of any one of the preceding claims, wherein the expanded cord blood or population of hematopoietic stem or progenitor cells is MGTA-456.

58. The method of any one of the preceding claims, wherein the hematopoietic stem or progenitor cells are expanded ex vivo by contacting the hematopoietic stem or progenitor cells with an aryl hydrocarbon receptor antagonist.

59. The method of any one of the preceding claims, wherein the aryl hydrocarbon receptor antagonist is SR-1 or a pharmaceutically acceptable salt, hydrate, or solvate thereof.

60. The method of any one of the preceding claims, wherein the aryl hydrocarbon receptor antagonist is compound 2 or a pharmaceutically acceptable salt, hydrate, or solvate thereof.

61. The method of any one of the preceding claims, wherein the aryl hydrocarbon receptor antagonist is a compound represented by formula (IV) 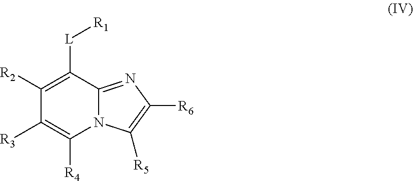
or a pharmaceutically acceptable salt, hydrate, or solvate thereof, wherein:
L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ—, —NR₇ₐC(S)(CR₈ₐR_{8b})ₙ—, —OC(O)(CR₈ₐR_{8b})ₙ—, —OC(S)(CR₈ₐR_{8b})—, —C(O)NR₇ₐ(CR₈ₐR_{8b})ₙ—, —C(S)NR₇ₐ(CR₈ₐR_{8b})ₙ—, —C(O)O(CR₈ₐR_{8b})ₙ—, —C(S)O(CR₈ₐR_{8b})ₙ—, —S(O)₂NR₇ₐ(CR₈ₐR_{8b})ₙ—, —NR₇ₐS(O)₂(CR₈ₐR_{8b})ₙ—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ and R_{8b} are each independently selected from the group consisting of hydrogen and optionally substituted C1-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{5b}, —NR₉ₐC(O)NR₉ₐR_{9b}, —C(O)R₉ₐ, —C(S)R₉ₐ, —S(O)₀₋₂R₉ₐ, —C(O)OR_{9b}, —C(S)OR₉ₐ, —C(O)NR₉ₐR_{9b}, —C(S)NR₉ₐR_{9b}, —NR₉ₐS(O)₂R_{8b}, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
R₂ is selected from the group consisting of hydrogen and optionally substituted C1-4 alkyl;
R₅ is selected from the group consisting of optionally substituted aryl, optionally substituted heteroaryl, optionally substituted cycloalkyl, and optionally substituted heterocycloalkyl;
R₄ is selected from the group consisting of hydrogen and optionally substituted C1-4 alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

62. The method of any one of the preceding claims, wherein the aryl hydrocarbon receptor antagonist is a compound represented by formula (V) 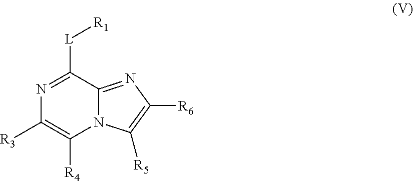
or a pharmaceutically acceptable salt, hydrate, or solvate thereof, wherein:
L is selected from the group consisting of —NR₇ₐ(CR₈ₐR_{8b})ₙ—, —O(CR₈ₐR_{8b})ₙ—, —C(O)(CR₈ₐR_{8b})ₙ—, —C(S)(CR₈ₐR_{8b})ₙ—, —S(O)₀₋₂(CR₈ₐR_{8b})ₙ—, —(CR₈ₐR_{8b})ₙ—, —NR₇ₐC(O)(CR₈ₐR_{8b})ₙ, —NR₇ₐC(S)(CR₈ₐR_{8b})ₙ, —OC(O)(CR₈ₐR_{8b})ₙ—, —OC(S)(CR₈ₐR_{8b})ₙ—, —C(O)NR₇ₐ(CR₈ₐR_{8b})ₙ—, —C(S)NR₇ₐ(CR₈ₐR_{8b})ₙ, —C(O)O(CR₈ₐR_{8b})ₙ—, —C(S)O(CR₈ₐR_{8b})ₙ—, —S(O)₂NR₇ₐ(CR₈ₐR_{8b})—, —NR₇ₐS(O)₂(CR₈ₐR_{8b})—, —NR₇ₐC(O)NR_{7b}(CR₈ₐR_{8b})ₙ—, and —NR₇ₐC(O)O(CR₈ₐR_{8b})ₙ—, wherein R₇ₐ, R_{7b}, R₈ₐ and R_{8b} are each independently selected from the group consisting of hydrogen and optionally substituted C1-4 alkyl, and each n is independently an integer from 2 to 6;
R₁ is selected from the group consisting of —S(O)₂NR₉ₐR_{9b}, —NR₉ₐC(O)R_{9b}, —NR₉ₐC(S)R_{9b}, —NR₉ₐC(O)NR_{9b}R_{9c}, —C(O)R₉ₐ, —C(S)R₉ₐ, —S(O)₀₋₂R₉ₐ, —C(O)OR₉ₐ, —C(S)OR₉ₐ, —C(O)NR₉ₐR_{9b}, —C(S)NR₉ₐR_{9b}, —NR₉ₐS(O)₂Rb, —NR₉ₐC(O)OR_{9b}, —OC(O)CR₉ₐR_{9b}R_{9c}, —OC(S)CR₉ₐR_{9b}R_{9c}, optionally substituted aryl, optionally substituted heteroaryl, optionally substituted cycloalkyl, and optionally substituted heterocycloalkyl, wherein R₉ₐ, R_{9b}, and R_{9c} are each independently selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl;
R₃ is selected from the group consisting of optionally substituted aryl, optionally substituted heteroaryl, optionally substituted cycloalkyl, and optionally substituted heterocycloalkyl;
R₄ is selected from the group consisting of hydrogen and optionally substituted C1-4 alkyl;
R₅ is selected from the group consisting of optionally substituted aryl, optionally substituted heteroaryl, optionally substituted alkyl, optionally substituted heteroalkyl, optionally substituted cycloalkyl, and optionally substituted heterocycloalkyl; and
R₆ is selected from the group consisting of hydrogen, optionally substituted aryl, optionally substituted heteroaryl, optionally substituted alkyl, optionally substituted heteroalkyl, optionally substituted cycloalkyl, and optionally substituted heterocycloalkyl.

63. The method of any one of the preceding claims, wherein the population, prior to expansion, comprises no more than 9×10⁷ CD34+ cells, no more than 8×10⁷ CD34+ cells, no more than 7×10⁷ CD34+ cells, no more than 6×10⁷ CD34+ cells, no more than 5×10⁷ CD34+ cells, no more than 9×10⁶ CD34+ cells, no more than 8×10⁸ CD34+ cells, no more than 7×10⁶ CD34+ cells, no more than 6×10⁸ CD34+ cells, no more than 5×10⁶ CD34+ cells, or no more than 1×10⁶ CD34+ cells.

64. The method of any one of the preceding claims, wherein the expanding comprises contacting the CD34+ cells with an aryl hydrocarbon receptor antagonist, preferably wherein the aryl hydrocarbon receptor antagonist is SR-1, compound 2, a compound represented by formula (IV), or a compound represented by formula (V).

65. The method of any one of the preceding claims, wherein prior to infusion into the patient, the hematopoietic stem or progenitor cells are mobilized and isolated from a donor.

66. The method of any one of the preceding claims, wherein the donor is a human.

67. The method of any one of the preceding claims, wherein the hematopoietic stem or progenitor cells are mobilized by contacting the hematopoietic stem or progenitor cells with a mobilizing amount of a CXCR4 antagonist and/or a CXCR2 agonist.

68. The method of any one of the preceding claims, wherein the CXCR4 antagonist is plerixafor.

69. The method of any one of the preceding claims, wherein the CXCR4 antagonist is BL-8040.

70. The method of any one of the preceding claims, wherein the CXCR2 agonist is Gro-s, Gro-β T, or a variant thereof.

71. The method of any one of the preceding claims, wherein the patient is a human.

72. The method of any one of the preceding claims, wherein the stem cell disorder is a hemoglobinopathy disorder.

73. The method of any one of the preceding claims, wherein the stem cell disorder is cancer.

74. The method of any one of the preceding claims wherein the stem cell disorder is an autoimmune disorder.

75. The method of any one of the preceding claims, wherein the hematopoietic stem or progenitor cells are autologous with respect to the patient.

76. The method of any one of the preceding claims, wherein the hematopoietic stem or progenitor cells are allogeneic with respect to the patient.

77. The method of any one of the preceding claims, wherein the hematopoietic stem or progenitor cells are HLA-matched with respect to the patient.

78. The method of any one of the preceding claims, wherein in the comparable method, the patient is conditioned prior to receiving the population of expanded hematopoietic stem or progenitor cells by a conditioning regimen comprising administering to the patient busulfan (Bu), fludarabine (Flu), and ATG (e.g., rATG).

79. The method of any one of the preceding claims, wherein in the comparable method, the patient is conditioned prior to receiving the population of expanded hematopoietic stem or progenitor cells by a conditioning regimen as described in Example 2.

80. The method of any one of the preceding claims, wherein the comparable method is substantially the same as the method other than that the patient is conditioned prior to receiving the population of expanded hematopoietic stem or progenitor cells by a conditioning regimen comprising administering to the patient busulfan (Bu) and fludarabine (Flu).

81. In some embodiments, the comparable method is substantially the same as the method other than that the patient is conditioned prior to receiving the population of expanded hematopoietic stem or progenitor cells by a conditioning regimen comprising administering to the patient busulfan (Bu), fludarabine (Flu), and ATG (e.g., rATG).

82. A kit comprising a plurality of hematopoietic stem or progenitor cells and a package insert, wherein the package insert instructs a user to perform the method of any one of the preceding claims.
